(19) United States
(12) Patent Application Publication
(10) Pub. No.: US 2011/0319400 A1
(43) Pub. Date: Dec. 29, 2011

(54) PROKINETICIN 1 RECEPTOR ANTAGONISTS FOR THE TREATMENT OF PAIN

(76) Inventors: Christopher M. Flores, Lansdale, PA (US); Paul R. Wade, Philadelphia, PA (US)

(21) Appl. No.: 13/169,552

(22) Filed: Jun. 27, 2011

Related U.S. Application Data
(60) Provisional application No. 61/359,124, filed on Jun. 28, 2010.

(51) Int. Cl. A61K 31/541; A61K 31/53; C07D 405/14; A61K 31/513; C07D 471/04; A61K 31/515; C07D 401/06; C07D 403/12; C07D 401/14; C07D 413/14; A61K 31/5377; C07D 417/14; C07D 409/14; A61P 29/00; C07D 401/12

(52) U.S. Cl. 514/227.8; 544/212; 514/245; 544/310; 514/274; 544/300; 514/270; 544/113; 514/236.2; 544/58.6

(57) ABSTRACT

Disclosed are compounds, compositions and methods for treating pain, including inflammatory, visceral, and acute pain. Such compounds are represented by Formula (I) as follows:

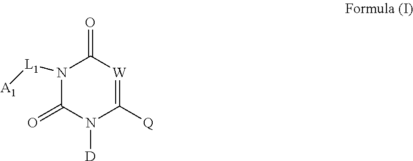

wherein A₁, L₁, D, and Q are defined herein.

### CROSS REFERENCE TO RELATED U.S. APPLICATION DATA

The present application is derived from and claims priority to provisional application U.S. Ser. No. 61/359,124, filed Jun. 28, 2010, which is herein incorporated by reference in its entirety.

The nonprovisional application entitled, Prokineticin 1 Receptor Antagonists, U.S. Nonprovisional application Ser. No. 11/647,091, filed on Dec. 28, 2006, is hereby incorporated by reference in its entirety.

### STATEMENT REGARDING FEDERALLY SPONSORED RESEARCH OR DEVELOPMENT

The research and development of the invention described below was not federally sponsored.

### FIELD OF THE INVENTION

The present invention is directed to the use of a compound of Formula (I), as herein defined, for the treatment, amelioration, and/or prevention of pain, including inflammatory pain, visceral pain, and acute pain, in a subject, including a mammal and/or human in need thereof.

### BACKGROUND OF THE INVENTION

Sensitization is an important property of pain signaling. Painful stimuli can induce central (spinal and supraspinal) and peripheral (nociceptor) sensitization. Both types of sensitization play a role in inflammatory diseases, the single greatest cause of chronic pain.

Prokineticin-1 and Prokineticin-2, PKR1 and PKR2 respectively, are naturally occurring peptide agonists of two G-protein-coupled receptors (GPCRs) and are expressed in neurons in the central nervous system (CNS) and peripheral nervous system. Many dorsal root ganglion cells expressing PKRs also express transient receptor potential vanilloid receptor-1 (TRPV1). It has been suggested that PKR1 plays a modulatory role in acute nociception and inflammatory pain through a pharmacological interaction with TRPV1 in nociceptor activation and sensitization. Moreover, PKR1 and PKR2 (Lin, D C H et al. J. Biol. Chem. 2002, 277, p 19276-19280) and their activation by peptides belonging to the Bv8/EG-VEGF (endocrine gland-derived vascular endothelial growth factor)-PK (prokineticin) family suggest an additional novel mechanism of peripheral nociceptor activation and sensitization (Negri et al., Br. J. Pharmacol. 2002, 146, p. 1147-1154).

It is suggested that prokineticin 1 receptor antagonists would be useful in the treatment and prevention of various mammalian pain states, including inflammatory pain, visceral pain, and acute pain.

It is an object of the present invention to provide prokineticin 1 receptor antagonists. It is also an object of the invention to provide a method of treating, ameliorating or preventing pain by the administration of a compound of Formula (I). And, it is an object of the invention to provide a pharmaceutical composition comprising a compound of Formula (I), useful for treating, ameliorating or preventing pain.

### SUMMARY OF THE INVENTION

The present invention is directed to a method for treating, ameliorating, or preventing pain; comprising, consisting of, and/or consisting essentially of administering to a subject in need thereof, a therapeutically effective amount of a compound of Formula (I)

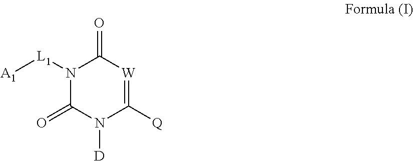

or an enantiomer, diastereomer, solvate, or pharmaceutically acceptable salt thereof;

wherein:
A₁ is CF₃, C₁₋₄alkoxy, aryl, aryloxy, benzofused heterocyclyl, or heteroaryl; wherein aryl, aryloxy, and heteroaryl are optionally substituted with pyrazol-1-yl or [1,2,3]thiadiazol-4-yl; or aryl, aryloxy, the benzo portion of benzofused heterocyclyl, and heteroaryl are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl; provided that A₁ is other than 3,5-di-t-butyl-phenyl;
L₁ is —(CH₂)ᵣ—, —CH₂C₂₋₄alkenyl-, or —CH₂CH₂X(CH₂)ₛ—, wherein L₁ is optionally substituted with one to two substituents independently selected from the group consisting of C₁₋₆alkyl, C₂₋₆alkenyl, C₂₋₆alkynyl, and halogen; and, r is an integer of 1 to 5; such that r is greater than or equal to 4 when A₁ is C₁₋₄alkoxy;
s is an integer of 1 to 3;
X is O or S;
D is —P-A₂;
wherein P is —(CH₂)₁₋₂- or —CH₂CH═CH— when A₂ is phenyl, benzofused heterocyclyl, heteroaryl, or C₃₋₈cycloalkyl; alternatively, P is —(CH₂)₃₋₆—, when A₂ is hydrogen, C₁₋₄alkoxy, or C₁₋₄alkoxycarbonyl; and wherein P is optionally substituted with one to two substituents independently selected from the group consisting of C₁₋₆alkyl, C₂₋₆alkenyl, C₂₋₆alkynyl, and halogen;
A₂ is hydrogen, C₁₋₄alkoxy, C₁₋₄alkoxycarbonyl, phenyl, benzofused heterocyclyl, heteroaryl, tetrahydro-pyranyl, piperidinyl, or C₃₋₈cycloalkyl; wherein phenyl, heteroaryl, the benzo portion of benzofused heterocyclyl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, N-isoindole-1,3-dione, C₁₋₆alkylthio, C₁₋₆alkylsulfonyl, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; such that no more than two substituents on A₂ are aryl(C₁₋₆)alkoxy, phenyl, N-isoindole-1,3-dione, or a non fused C₃₋₆cycloalkyloxy;
   provided that A₂ is other than 3,5-di-t-butyl-phenyl;
W is N or C(R_{W}); wherein R_{W} is H or C₁₋₂alkyl;
Q is selected from the group consisting of (a) to (g), wherein

(a) is —NH(CH₂)₂—Ar₁ wherein Ar₁ is pyridinyl optionally substituted with one to three C₁₋₄alkyl substituents or a substituent selected from the group consisting of C₁₋₄alkoxy and amino;

provided that when Ar₁ is an unsubstituted pyridin-3-yl or unsubstituted pyridin-4-yl, and A₂ is 4-methoxy-phenyl, A₁ is other than unsubstituted phenyl or 3,4-dichloro-phenyl;

(b) is —NHCH(R_{z})—Ar₂ wherein R_{z} is H or C₁₋₃alkyl; Ar₂ is pyridinyl, pyrimidinyl, pyrazinyl,

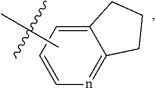

1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; and wherein Ar₂ is optionally substituted with one to three substituents independently selected from the group consisting of C₁₋₄alkyl, trifluoromethyl, hydroxyl-C₁₋₄alkyl, amino(C₁₋₄)alkyl, (C₁₋₄alkyl)amino-(C₁₋₄)alkyl, di(C₁₋₄alkyl)amino-(C₁₋₄)alkyl, C₁₋₄alkoxy, C₃₋₈cycloalkylamino, amino, (C₁₋₆alkyl)amino, and di(C₁₋₆alkyl)amino; or Ar₂ is optionally substituted with one amino group and three substituents independently selected from the group consisting of C₁₋₄alkyl and C₁₋₄alkoxy;

wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, C₁₋₄alkylthio, hydroxy, a 5 to 6 membered heteroaryl, or a 5 to 6 membered heterocyclyl; wherein a nitrogen atom of the 5 to 6 membered heterocyclyl is optionally substituted with a C₁₋₄alkyl substituent;

and wherein pyridin-2-yl and pyridin-3-yl are optionally further substituted with N-pyrrolidinyl, N-piperazinyl, N-piperidinyl, N-morpholinyl, N-thiomorpholinyl, —CH₂—O—CH₂—PH, and phenyl; wherein the phenyl substituent of pyridin-2-yl and pyridin-3-yl is optionally substituted with one to three substituents independently selected from the group consisting of C₁₋₄alkyl, C₁₋₄alkoxy, and halogen;

provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;

provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;

provided that when Q is —NHCH₂(2-amino-pyridin-3-yl), and A₁ is benzotriazol-1-yl, A₂ is other than 4-difluoromethoxy-phenyl;

provided that when Q is —NHCH₂(2-amino-pyridin-3-yl), L₁ is —(CH₂)₃—, and A₁ is pyrrol-1-yl, A₂ is other than 4-methoxy-phenyl;

provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;

provided that when Q is —NHCH₂(2-amino-pyridin-3-yl), and A₁ is 4-fluoro-phenyl, A₂ is other than 4-fluoro-phenyl;

provided that when Q is —NHCH₂(6-amino-pyridin-2-yl), and A₁ is 4-fluoro-phenyl, A₂ is other than 4-trifluoromethoxy-phenyl;

provided that when Q is —NHCH₂(6-methyl-pyridin-2-yl), and A₁ is 4-methoxy-phenyl, A₂ is other than 4-methoxy-phenyl;

provided that when Q is —NHCH₂(imidazo[1,2-a]pyridinyl), and A₁ is 4-fluoro-phenyl, A₂ is other than 4-methoxy-phenyl;

provided that when Q is —NHCH₂(pyridin-4-yl), and A₁ is unsubstituted phenyl or 3,4-dichloro-phenyl, A₂ is other than 4-methoxy-phenyl;

provided that when Q is —NHCH₂(4,6-dimethyl-pyridin-3-yl), and A₁ is 4-methoxy-phenyl, —P-A₂ is other than —(CH₂)₅-methoxy;

provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;

provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;

provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;

and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;

(c) is —CH₂NHCH₂—Ar₃, wherein W is N or CH, and Ar₃ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and that the point of attachment to quinolinyl is at the 2, 3, or 4-position; wherein Ar₃ is optionally substituted with one to three substituents independently selected from the group consisting of C₁₋₄alkyl, amino(C₁₋₄alkyl, (C₁₋₄alkyl)amino-(C₁₋₄alkyl, di(C₁₋₄alkyl)amino-(C₁₋₄)alkyl, C₁₋₄alkoxy, amino, (C₁₋₆alkyl)amino, and di(C₁₋₆)alkyl)amino;

and wherein the C₁₋₆alkyl group of (C₁₋₆)alkyl)amino and di(C₁₋₆)alkyl)amino is optionally substituted with amino, (C₁₋₄)alkyl)amino, di(C₁₋₄)alkyl)amino, C₃₋₈cycloalkylamino, C₁₋₄alkoxy, or hydroxy;

(d) is —(CH₂)₂—Ar₄, wherein Ar₄ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₄ is optionally substituted with one to three substituents independently selected from the group consisting of C₁₋₄alkyl, amino(C₁₋₄alkyl, (C₁₋₄)alkyl)amino-(C₁₋₄)alkyl, di(C₁₋₄alkyl)amino-(C₁₋₄)alkyl, C₁₋₄alkoxy, amino, (C₁₋₆alkyl)amino, di(C₁₋₆alkyl)amino, halogen, and aminocarbonyl;

and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;

(e) is —CH═CH—Ar₅; wherein Ar₅ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₅ is optionally substituted with one to three substituents independently selected from the group consisting of C₁₋₄alkyl, amino(C₁₋₄alkyl, (C₁₋₄alkyl)amino-(C₁₋₄)alkyl, di(C₁₋₄alkyl)amino-(C₁₋₄)alkyl, C₁₋₄alkoxy, amino, (C₁₋₆alkyl)amino, di(C₁₋₆alkyl)amino, halogen, and aminocarbonyl;

and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;

(f) is —O—CH(R₁)—Ar₆ when W is CH; or, (f) is —S—CH(R₁)—Ar₆ and W is N or CH; wherein R₁ is hydrogen or C₁₋₄alkyl, and Ar₆ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;

wherein Ar₆ is optionally substituted with one to three substituents independently selected from the group consisting of C₁₋₄alkyl, amino(C₁₋₄alkyl, (C₁₋₄alkyl)amino-(C₁₋₄)alkyl, di(C₁₋₄alkyl)amino-(C₁₋₄)alkyl, C₁₋₄alkoxy, amino, (C₁₋₆alkyl)amino, di(C₁₋₆alkyl)amino, halogen, and aminocarbonyl;

and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;

provided that when Q is —O—CH(R₁)—Ar₆, A₁ and A₂ are 4-methoxy-phenyl, and R₁ is hydrogen, Ar₆ is other than unsubstituted pyridin-2-yl or 2-amino-pyridin-4-yl;

and

(g) is —X₁—(CH(Rₓ))2—Ar₇ when W is CH; wherein X₁ is O or S, Rₓ is H or C₁₋₄alkyl, and Ar₇ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;

wherein Ar₇ is optionally substituted with one to three substituents independently selected from the group consisting of C₁₋₄alkyl, amino(C₁₋₄alkyl, (C₁₋₄alkyl)amino-(C₁₋₄alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl; and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;

provided that when Q is —O (CH₂)₂—Ar₇ and A₁ and A₂ are 4-methoxy-phenyl, Ar₇ is other than unsubstituted pyridin-2-yl or unsubstituted pyridin-3-yl;

wherein a nitrogen atom of Ar₁, Ar₂, Ar₃, Ar₄, Ar₅, Ar₆, and Ar₇ is optionally substituted with oxo.

The present invention is further directed to the use of a compound of Formula (I) as herein defined for the preparation of a medicament or a pharmaceutical composition for the treatment, amelioration and/or prevention of pain, including inflammatory, visceral, and acute pain, in a subject in need thereof.

### DETAILED DESCRIPTION OF THE INVENTION

As used herein, the following terms are intended to have the following meanings:

“C_{a-b}” (where a and b are integers) refers to a radical containing from a to b carbon atoms inclusive. For example, C₁₋₃ denotes a radical containing 1, 2 or 3 carbon atoms.

With reference to substituents, the term “independently” means that when more than one of such substituent is possible, such substituents may be the same or different from each other. Therefore, designated numbers of carbon atoms (e.g. C₁₋₈) shall refer independently to the number of carbon atoms in an alkyl or cycloalkyl moiety or to the alkyl portion of a larger substituent in which alkyl appears as its prefix root.

As used herein, unless otherwise noted, “alkyl” whether used alone or as part of a substituent group refers to straight and branched carbon chains having 1 to 8 carbon atoms or any number within this range. The term “alkoxy” refers to an -Oalkyl substituent group, wherein alkyl is as defined supra. Similarly, the terms “alkenyl” and “alkynyl” refer to straight and branched carbon chains having 2 to 8 carbon atoms or any number within this range, wherein an alkenyl chain has at least one double bond in the chain and an alkynyl chain has at least one triple bond in the chain. An alkyl and alkoxy chain may be substituted on a carbon atom. In substituent groups with multiple alkyl groups such as (C₁₋₆alkyl)₂-amino- the C₁₋₆alkyl groups of the dialkylamino may be the same or different.

“Halogenated alkyl” refers to a saturated branched or straight chain alkyl radical derived by removal of 1 hydrogen atom from the parent alkyl; the parent alkyl chain contains from 1 to 8 carbon atoms with 1 or more hydrogen atoms substituted with halogen atoms up to and including substitution of all hydrogen atoms with halogen. Preferred halogenated alkyl groups include include trifluoromethyl substituted alkyls and perfluorinated alkyls; more preferred fluorinated alkyls include trifluoromethyl.

“Halogenated alkoxy” refers to a radical derived from a halogenated alkyl, radical attached to an oxygen atom with the oxygen atom having one open valence for attachment to a parent structure.

The term “cycloalkyl” refers to saturated or partially unsaturated, moncyclic or polycyclic hydrocarbon rings of from 3 to 20 carbon atom members (preferably from 3 to 14 carbon atom members). Examples of such rings include, and are not limited to, cyclopropyl, cyclobutyl, cyclopentyl, cyclohexyl, cycloheptyl or adamantyl. The term cycloalkyl includes a cycloalkyl ring fused to a benzene ring (benzo fused cycloalkyl), a 5 or 6 membered heteroaryl ring (containing one of O, S or N and, optionally, one additional nitrogen) to form a heteroaryl fused cycloalkyl.

The term “heterocyclyl” refers to a nonaromatic cyclic ring of 5 to 10 members in which 1 to 4 members are nitrogen or a nonaromatic cyclic ring of 5 to 10 members in which zero, one or two members are nitrogen and up to two members is oxygen or sulfur; wherein, optionally, the ring contains zero, one or two unsaturated bonds. The term heterocyclyl includes a heterocyclyl ring fused to a benzene ring (benzo fused heterocyclyl), a 5 or 6 membered heteroaryl ring (containing one of O, S or N and, optionally, one additional nitrogen), a 5 to 7 membered cycloalkyl or cycloalkenyl ring, a 5 to 7 membered heterocyclyl ring (of the same definition as above but absent the option of a further fused ring) or fused with the carbon of attachment of a cycloalkyl, cycloalkenyl or heterocyclyl ring to form a spiro moiety. For instant compounds of the invention, the carbon atom ring members that form the heterocyclyl ring are fully saturated. Other compounds of the invention may have a partially saturated heterocyclyl ring. Additionally, heterocyclyl includes a heterocyclic ring bridged to form bicyclic rings. Preferred partially saturated heterocyclyl rings may have from one to two double bonds. Such compounds are not considered to be fully aromatic and are not referred to as heteroaryl compounds. Examples of heterocyclyl groups include, and are not limited to, pyrrolinyl (including 2H-pyrrole, 2-pyrrolinyl or 3-pyrrolinyl), pyrrolidinyl, 2-imidazolinyl, imidazolidinyl, 2-pyrazolinyl, pyrazolidinyl, piperidinyl, morpholinyl, thiomorpholinyl and piperazinyl.

The term “aryl” refers to an unsaturated, aromatic monocyclic ring of 6 carbon members or to an unsaturated, aromatic polycyclic ring of from 10 to 14 carbon members. Examples of such aryl rings include, and are not limited to, phenyl, naphthalenyl or anthracenyl. Preferred aryl groups for the practice of this invention are phenyl and naphthalenyl.

The term “heteroaryl” refers to an aromatic ring of 5 or 6 members wherein the ring consists of carbon atoms and has at least one heteroatom member. Suitable heteroatoms include nitrogen, oxygen or sulfur. In the case of 5 membered rings, the heteroaryl ring contains one member of nitrogen, oxygen or sulfur and, in addition, may contain up to three additional nitrogens. In the case of 6 membered rings, the heteroaryl ring may contain from one to three nitrogen atoms. For the case wherein the 6 membered ring has three nitrogens, at most two nitrogen atoms are adjacent. The term heteroaryl includes a heteroaryl ring fused to a benzene ring (benzo fused heteroaryl) such as

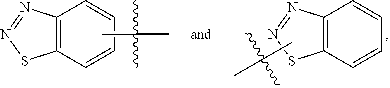

a 5 or 6 membered heteroaryl ring (containing one of O, S or N and, optionally, one additional nitrogen), a 5 to 7 membered cycloalkyl ring or a 5 to 7 membered heterocyclic ring (as defined supra but absent the option of a further fused ring). For such compounds in which the heteroaryl ring is fused to a moiety as described above, the point of attachment is through the heteroaryl ring portion of the compound. Examples of heteroaryl groups include, and are not limited to, furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl or pyrazinyl; fused heteroaryl groups include indolyl, isoindolyl, indolinyl, benzofuryl, benzothienyl, indazolyl, benzimidazolyl, benzthiazolyl, benzoxazolyl, benzisoxazolyl, benzothiadiazolyl, benzotriazolyl, quinolizinyl, quinolinyl, isoquinolinyl or quinazolinyl.

The term “arylalkyl” means an alkyl group substituted with an aryl group (e.g., benzyl, phenethyl). Similarly, the term “arylalkoxy” indicates an alkoxy group substituted with an aryl group (e.g., benzyloxy).

The term “halogen” refers to fluorine, chlorine, bromine and iodine. Substituents that are substituted with multiple halogens are substituted in a manner that provides compounds, which are stable.

The term “oxo” whether used alone or as part of a substituent group refers to an O═ to either a carbon or a sulfur atom. For example, phthalimide and saccharin are examples of compounds with oxo substituents.

Whenever the term “alkyl” or “aryl” or either of their prefix roots appear in a name of a substituent (e.g., arylalkyl, alkylamino) it shall be interpreted as including those limitations given above for “alkyl” and “aryl.” Designated numbers of carbon atoms (e.g., C₁-C₆) shall refer independently to the number of carbon atoms in an alkyl moiety or to the alkyl portion of a larger substituent in which alkyl appears as its prefix root. For alkyl, and alkoxy substituents the designated number of carbon atoms includes all of the independent member included in the range specified individually and all the combination of ranges within in the range specified. For example C₁₋₆ alkyl would include methyl, ethyl, propyl, butyl, pentyl and hexyl individually as well as sub-combinations thereof (e.g., C₁₋₂, C₁₋₃, C₁₋₄, C₁₋₅, C₂₋₆, C₃₋₆, C₄₋₆, C₅₋₆, C₂₋₅, etc.).

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, the term “acyl” refers to alkylcarbonyl substituents.

As used herein, positions on a tetrahydro[1,8]naphthyridinyl substituent will be referred to using the following numbering system:

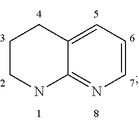

however, one of ordinary skill in the art will recognize that the numbering of the tetrahydro[1,8]naphthyridinyl ring system in a compound described herein, such as those shown in a specific example, may differ from that shown above.

Throughout this disclosure, the terminal portion of the designated side chain is described first, followed by the adjacent functionality toward the point of attachment. Thus, for example, a “phenyl(C₁₋₆)alkylaminocarbonyl(C₁₋₆)alkyl” substituent refers to a group of the formula

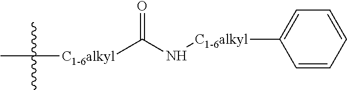

Unless otherwise noted, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “subject” as used herein, refers to an animal, preferably a mammal, most preferably a human, who has been the object of treatment, observation or experiment.

The term “therapeutically effective amount” means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease, syndrome, condition or disorder being treated.

The term “composition” is intended to encompass a product comprising the specified ingredients in therapeutically effective amounts, as well as any product that results, directly or indirectly, from combinations of the specified ingredients in the specified amounts.

As used herein, unless otherwise noted, the terms “treating”, “treatment”, “ameliorating” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the terms “preventing” and “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical professional to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

As used herein, unless otherwise noted, the term “antagonist” is used to refer to a compound capable of producing, depending on the circumstance, a functional antagonism of the prokinetin receptor 1, including, but not limited to, competitive antagonists, non-competitive antagonists, desensitizing agonists, and partial agonists.

As used herein, unless otherwise noted, the term “affect” or “affected” (when referring to a disease, syndrome, condition or disorder that is affected by inhibition of MGL) shall imply a reduction in the frequency and/or severity of one or more symptoms or manifestations of said disease, syndrome, condition or disorder; and/or imply the prevention of the development of one or more symptoms or manifestations of said disease, syndrome, condition or disorder or the development of the disease, condition, syndrome or disorder.

The compounds of Formula (I) are useful in methods for treating, ameliorating and/or preventing pain or a disease, a syndrome, a condition or a disorder that causes such pain, by the antagonism of prokineticin 1 receptor. Such methods comprise, consist of and/or consist essentially of administering to a subject, including an animal, a mammal, and a human in need of such treatment, amelioration and/or prevention, a therapeutically effective amount of a compound of Formula (I), or an enantiomer, diastereomer, solvate or pharmaceutically acceptable salt thereof. More particularly, the compounds of Formula (I) are useful for treating, ameliorating and/or preventing inflammatory pain, visceral pain and/or acute pain, comprising administering to a subject in need thereof a therapeutically effective amount of a compound of Formula (I), as herein defined.

Examples of inflammatory pain include pain due to a disease, condition, syndrome or disorder, including inflammatory bowel disease, visceral pain, migraine, post operative pain, osteoarthritis, rheumatoid arthritis, back pain, lower back pain, joint pain, abdominal pain, chest pain, labor pain, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

The term visceral pain, as used herein, refers to pain caused by inflammation of serous surfaces, distention of viscera and inflammation or compression of peripheral nerves. Examples of visceral pain include, but are not limited to, abdominal pain, chest pain, pelvic pain, including vulvodynia as well as pain associated with labor or menstruation, and/or pain associated with inflammatory bowel disease, irritable bowel syndrome, neurogenic bladder, interstitial cystitis, cholecystitis, pancreatitis and urinary tract infection.

Acute pain, as used herein, refers to pain that comes on quickly, can be severe, but is of relatively short duration. Examples of acute pain include, but are not limited to, post-operative pain, post-surgical pain, toothache, burn, sunburn, insect/animal bites and stings, headache and/or any pain associated with acute trauma or injury.

In an embodiment, the present invention is directed to a method for treating, ameliorating, or preventing pain; comprising, consisting of, and/or consisting essentially of administering to a subject in need thereof, a therapeutically effective amount of a compound of Formula (I)

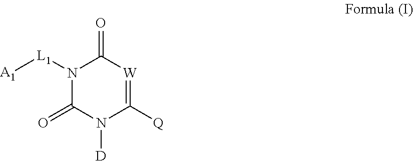

or an enantiomer, diastereomer, solvate, or pharmaceutically acceptable salt thereof;

wherein:
(i) A₁ is aryl, heteroaryl, or a benzofused heterocyclyl selected from the group consisting of benzo[1,3]dioxalyl and 2,3-dihydro-benzofuranyl; wherein aryl and heteroaryl are optionally substituted with one to three substituents independently selected from the group consisting of C₁₋₄alkyl, C₁₋₄alkoxy, nitro, fluoro, chloro, iodo, halogenated C₁₋₄alkyl, halogenated C₁₋₄alkoxy, and C₁₋₄alkylthio; provided that A₁ is other than 3,5-di-t-butyl-phenyl;
(ii) A₁ is aryl, heteroaryl, or a benzofused heterocyclyl selected from the group consisting of benzo[1,3]dioxalyl and 2,3-dihydro-benzofuranyl; wherein aryl and heteroaryl are optionally substituted with one to three substituents independently selected from the group consisting of C₁₋₃alkyl, methoxy, fluoro, chloro, trifluoromethyl, trifluoromethoxy, and methylthio;
(iii) A₁ is substituted phenyl, heteroaryl, or a benzofused heterocyclyl selected from the group consisting of benzo[1,3]dioxalyl and 2,3-dihydro-benzofuranyl; wherein substituted phenyl and heteroaryl are optionally substituted with one to three substituents independently selected from the group consisting of C₁₋₃alkyl, methoxy, fluoro and methylthio;
(iv) A₁ is substituted phenyl, benzotriazolyl, benzofuranyl, benzo[1,3]dioxalyl, or 2,3-dihydro-benzofuranyl; wherein phenyl is substituted with, and benzotriazolyl and benzofuranyl are optionally substituted with, one to three substituents independently selected from the group consisting of C₁₋₄alkyl, C₁₋₄alkoxy, nitro, fluoro, chloro, iodo, halogenated C₁₋₄alkyl, halogenated C₁₋₄alkoxy, and C₁₋₄alkylthio; provided that A₁ is other than 3,5-di-t-butyl-phenyl;
(v) A₁ is substituted phenyl, benzotriazolyl, benzofuranyl, benzo[1,3]dioxalyl, or 2,3-dihydro-benzofuranyl; wherein phenyl is substituted at the 4-position with methoxy, fluoro, or methylthio; and wherein A₁ other than substituted phenyl is optionally substituted with one to two substituents independently selected from the group consisting of methyl, methoxy, fluoro and methylthio;
(vi) L₁ is —(CH₂)ᵣ—, wherein L₁ is optionally substituted with one to two substituents independently selected from the group consisting of C₁₋₄alkyl and C₂₋₄alkenyl, and r is 1 or 2;
(vii) L₁ is —CH₂—;
(viii) P is —(CH₂)₁₋₂— when A₂ is phenyl, benzofused heterocyclyl, heteroaryl, or C₃₋₈cycloalkyl; alternatively, P is —(CH₂)₄₋₆—, when A₂ is hydrogen, C₁₋₄alkoxy, or C₁₋₄alkoxycarbonyl;
(ix) P is —CH₂— when A₂ is phenyl, benzofused heterocyclyl, heteroaryl, or C₃₋₈cycloalkyl; alternatively, P is —(CH₂)₄₋₆—, when A₂ is hydrogen, C₁₋₄alkoxy, or C₁₋₄alkoxycarbonyl;
(x) A₂ is hydrogen, C₁₋₄alkoxy, C₁₋₄alkoxycarbonyl, phenyl, benzofused heterocyclyl, heteroaryl other than pyridin-4-yl, or C₃₋₈cycloalkyl; wherein phenyl, heteroaryl and C₃₋₈cycloalkyl are optionally substituted with one to two substituents independently selected from the group consisting of C₁₋₆alkyl, C₁₋₆alkoxy, fluoro, chloro, halogenated C₁₋₆alkoxy, phenyl, N-isoindole-1,3-dione, C₁₋₆alkylthio, C₁₋₆alkylsulfonyl, C₁₋₆alkoxycarbonyl, nitro, hydroxy, and C₁₋₆alkylcarbonylamino; such that no more than one substituent on A₂ is phenyl or N-isoindole-1,3-dione; and provided that A₂ is other than 3,5-di-t-butyl-phenyl;
(xi) A₂ is C₁₋₄alkoxy, phenyl, benzofused heterocyclyl, or a heteroaryl other than pyridin-4-yl; wherein phenyl and heteroaryl are optionally substituted with one to two substituents independently selected from the group consisting of C₁₋₄)alkyl, C₁₋₄alkoxy, fluoro, chloro, halogenated C₁₋₄alkoxy, N-isoindole-1,3-dione, C₁₋₄alkylthio, C₁₋₄alkylsulfonyl, C₁₋₄alkoxycarbonyl, nitro, hydroxy, and C₁₋₄alkylcarbonylamino; such that no more than one substituent on A₂ is N-isoindole-1,3-dione; and provided that A₂ is other than 3,5-di-t-butyl-phenyl;
(xii) A₂ is C₁₋₄alkoxy, phenyl, benzofused heterocyclyl, or a heteroaryl other than pyridin-4-yl; wherein phenyl and heteroaryl are optionally substituted with one to two substituents independently selected from the group consisting of C₁₋₄alkoxy, fluoro, halogenated C₁₋₄alkoxy, C₁₋₄alkylthio, C₁₋₄alkylsulfonyl, C₁₋₄alkoxycarbonyl, nitro, and hydroxy;
(xiii) A₂ is C₁₋₄alkoxy, phenyl, 2,3-dihydro-benzofuranyl, indolyl, benzofuranyl, pyridin-3-yl, or benzothiophenyl; wherein A₂ other than C₁₋₄alkoxy is optionally substituted with one to two substituents independently selected from the group consisting of C₁₋₄alkoxy, fluoro, fluorinated C₁₋₄alkoxy, C₁₋₄alkylthio, C₁₋₄alkylsulfonyl, C₁₋₄alkoxycarbonyl, nitro, and hydroxy;
(xiv) W is N or CH;
(xv) W is N;
(xvi) Q is selected from the group consisting of (a)-(g) wherein:
   (a) is —NH(CH₂)₂—Ar₁ wherein Ar₁ is pyridinyl substituted with one to three C₁₋₄alkyl substituents or a substituent selected from the group consisting of C₁₋₄alkoxy and amino;
   (b) is —NHCH₂—Ar₂ wherein Ar₂ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; and wherein Ar₂ is optionally substituted with one to three substituents independently selected from the group consisting of C₁₋₄alkyl, trifluoromethyl, C₁₋₄alkoxy, amino, (C₁₋₆alkyl)amino, and di(C₁₋₆)alkyl)amino;
   wherein the C₁₋₆)alkyl group of (C₁₋₆alkyl)amino and di(C₁₋₆alkyl)amino is optionally substituted with (C₁₋₄alkyl)amino, di(C₁₋₄alkyl)amino, C₁₋₄alkoxy, C₁₋₄alkylthio, hydroxy, a 5 to 6 membered heteroaryl, or a 5 to 6 membered heterocyclyl; wherein a nitrogen atom of the 5 to 6 membered heterocyclyl is optionally substituted with a C₁₋₄alkyl substituent;
   and wherein pyridin-2-yl and pyridin-3-yl are optionally further substituted with N-pyrrolidinyl, N-piperazinyl, N-piperidinyl, N-morpholinyl, N-thiomorpholinyl, and phenyl; wherein the phenyl substituent of pyridin-2-yl and pyridin-3-yl is optionally substituted with one to three substituents independently selected from the group consisting of C₁₋₄alkyl, C₁₋₄alkoxy, and halogen;
   provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;
   provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), L₁ is —(CH₂)₃—, and A₁ is pyrrol-1-yl, A₂ is other than 4-methoxy-phenyl;
   provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is unsubstituted phenyl or 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
   provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
   provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
   and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
   (c) is —CH₂NHCH₂—Ar₃, wherein W is N or CH, and Ar₃ is pyridinyl optionally substituted with amino;
   (d) is —(CH₂)₂—Ar₄, wherein Ar₄ is pyridinyl, or pyrimidinyl; wherein Ar₄ is optionally substituted with one to two substituents independently selected from the group consisting of C₁₋₄alkyl, C₁₋₄alkoxy, amino, (C₁₋₆alkyl)amino, and di(C₁₋₆)alkyl)amino;
   (e) is —CH═CH-pyridinyl;
   (f) is —O—CH(R₁)—Ar₆ when W is CH; or, (f) is —S—CH(R₁)—Ar₆ and W is N or CH; wherein R₁ is hydrogen or C₁₋₄alkyl, and Ar₆ is pyridinyl or pyrimidinyl; wherein Ar₆ is optionally substituted with one to three substituents independently selected from the group consisting of C₁₋₄alkyl, C₁₋₄alkoxy, amino, (C₁₋₆alkyl)amino, di(C₁₋₆alkyl)amino, halogen, and aminocarbonyl;
   and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
   provided that when Q is —O—CH(R₁)—Ar₆, A₁ and A₂ are 4-methoxy-phenyl, and R₁ is hydrogen, Ar₆ is other than unsubstituted pyridin-2-yl or 2-amino-pyridin-4-yl; and
   (g) is —X₁—(CH(Rₓ))₂—Ar₇ and W is CH; wherein X₁ is O, Rₓ is H, and Ar₇ is pyridinyl or pyrimidinyl; wherein Ar₇ is optionally substituted with one to two substituents independently selected from the group consisting of C₁₋₄alkyl, C₁₋₄alkoxy, amino, (C₁₋₆alkyl)amino, and di(C₁₋₆alkyl)amino;
   provided that when Q is —O (CH₂)₂—Ar₇ and A₁ and A₂ are 4-methoxy-phenyl, Ar₇ is other than unsubstituted pyridin-2-yl or unsubstituted pyridin-3-yl;
   wherein a nitrogen atom of Ar₁, Ar₂, Ar₃, Ar₄, Ar₆, and Ar₇ is optionally substituted with oxo;
(xvii) Q is selected from the group consisting of (b) and (d) wherein:
   (b) is —NHCH₂—Ar₂ wherein Ar₂ is pyridinyl, pyrimidinyl, or quinolinyl; such that the point of attachment to quinolinyl is at the 2, 3, or 4-position; and wherein Ar₂ is optionally substituted with one to three substituents independently selected from the group consisting of C₁₋₄alkyl, trifluoromethyl, C₁₋₄alkoxy, amino, (C₁₋₄alkyl)amino, and di(C₁₋₄alkyl)amino;
   wherein the C₁₋₄alkyl group of (C₁₋₄alkyl)amino and di(C₁₋₄alkyl)amino is optionally substituted with (C₁₋₄alkyl)amino, di(C₁₋₄alkyl)amino, C₁₋₄-alkoxy, C₁₋₄alkylthio, hydroxy, a 5 to 6 membered heteroaryl, or a 5 to 6 membered heterocyclyl;
   and wherein pyridin-2-yl and pyridin-3-yl are optionally further substituted with N-morpholinyl;
   provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;
   provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), L₁ is —(CH₂)₃—, and A₁ is pyrrol-1-yl, A₂ is other than 4-methoxy-phenyl;
   provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is unsubstituted phenyl or 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
   provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
   provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
   and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
   (d) is —(CH₂)₂—Ar₄ and W is CH; wherein Ar₄ is pyridinyl is optionally substituted with one to two substituents independently selected from the group consisting of C₁₋₄alkyl, C₁₋₄alkoxy, amino, (C₁₋₆alkyl)amino, and di(C₁₋₆)alkyl)amino;
   wherein a nitrogen atom of Ar₂ and Ar₄ is optionally substituted with oxo;
(xviii) Q is selected from the group consisting of (b) and (d) wherein:
   (b) is —NHCH₂—Ar₂ wherein Ar₂ is pyridin-2-yl, pyridin-3-yl, or pyrimidinyl; wherein Ar₂ is optionally substituted with one to three substituents independently selected from the group consisting of C₁₋₄alkyl, trifluoromethyl, C₁₋₄alkoxy, amino, and (C₁₋₄alkyl)amino;
   wherein the C₁₋₄alkyl group of (C₁₋₄alkyl)amino is optionally substituted with di(C₁₋₄alkyl)amino, C₁₋₄alkoxy, or hydroxy;
   and wherein pyridin-2-yl and pyridin-3-yl are optionally further substituted with N-morpholinyl;
   provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;
   provided that when Q is —NHCH₂(2-amino-pyridin-3-yl), L₁ is —(CH₂)₂— or —(CH₂)₅—, and A₁ is methoxy, A₂ is 4-difluoromethoxy-phenyl or 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), L₁ is —(CH₂)₃—, and A₁ is pyrrol-1-yl, A₂ is other than 4-methoxy-phenyl;
   provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is unsubstituted phenyl or 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
   provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
   provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
   and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
   (d) is —(CH₂)₂—Ar₄ and W is CH; wherein Ar₄ is pyridinyl is optionally substituted with amino;
   wherein a nitrogen atom of Ar₂ and Ar₄ is optionally substituted with oxo;
(xviv) Q is —NHCH₂—Ar₂ wherein Ar₂ is unsubstituted pyridin-2-yl, 4,6-dimethyl-pyridin-3-yl, 2-amino-pyridin-3-yl, or 2-((C₁₋₄alkyl)amino)-pyridin-3-yl;
   wherein the C₁₋₄alkyl group of (C₁₋₄alkyl)amino is optionally substituted with di(C₁₋₄alkyl)amino, C₁₋₄alkoxy, or hydroxy;
   and wherein 2-amino-pyridin-3-yl is optionally further substituted with 4,6-dimethyl or 4-methoxy;
   provided that when Q is —NHCH₂(2-amino-pyridin-3-yl), and A₁ is pyridin-4-yl, 4-t-butyl-phenyl, 3,4-dichloro-phenyl, or 4-methanesulfonyl-phenyl, A₂ is other than 4-methoxy-phenyl;
   provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), L₁ is —(CH₂)₃—, and A₁ is pyrrol-1-yl, A₂ is other than 4-methoxy-phenyl;
   provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is unsubstituted phenyl or 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
   provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
   provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
   and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
   wherein a nitrogen atom of Ar₂ and Ar₄ is optionally substituted with oxo;
      and combinations of (i) through (xviv) above.

In an embodiment, the present invention is directed to a method for treating, ameliorating, or preventing pain comprising, consisting of, and/or consisting essentially of administering to a subject in need thereof, a therapeutically effective amount of a compound of Formula (I):

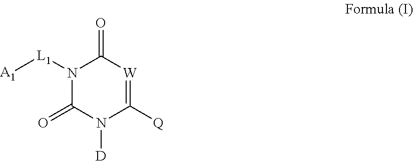

or an enantiomer, diastereomer, solvate, or pharmaceutically acceptable salt thereof;

wherein:
A₁ is CF₃, aryl, heteroaryl, or a benzofused heterocyclyl selected from the group consisting of benzo[1,3]dioxalyl and 2,3-dihydro-benzofuranyl; wherein aryl and heteroaryl are optionally substituted with one to three substituents independently selected from the group consisting of C₁₋₄alkyl, C₁₋₄alkoxy, nitro, fluoro, chloro, iodo, halogenated C₁₋₄alkyl, halogenated C₁₋₄alkoxy, and C₁₋₄alkylthio; provided that A₁ is other than 3,5-di-t-butyl-phenyl;
L₁ is —(CH₂)ᵣ—, wherein L₁ is optionally substituted with one to two substituents independently selected from the group consisting of C₁₋₄alkyl and C₂₋₄alkenyl and r is 1 or 2;
D is —P-A₂;
wherein P is —(CH₂)₁₋₂— when A₂ is phenyl, benzofused heterocyclyl, heteroaryl, or C₃₋₈cycloalkyl; alternatively, P is —(CH₂)₄₋₆—, when A₂ is hydrogen, C₁₋₄alkoxy, or C₁₋₄alkoxycarbonyl;
A₂ is hydrogen, C₁₋₄alkoxy, C₁₋₄alkoxycarbonyl, phenyl, benzofused heterocyclyl, heteroaryl other than pyridin-4-yl, tetrahydro-pyranyl, piperidinyl, or C₃₋₈cycloalkyl; wherein phenyl, heteroaryl and C₃₋₈cycloalkyl are optionally substituted with one to two substituents independently selected from the group consisting of C₁₋₆)alkyl, C₁₋₆alkoxy, fluoro, chloro, halogenated C₁₋₆alkoxy, phenyl, N-isoindole-1,3-dione, C₁₋₆alkylthio, C₁₋₆alkylsulfonyl, C₁₋₆alkoxycarbonyl, nitro, hydroxy, and C₁₋₆alkylcarbonylamino; provided that no more than one substituent on A₂ is phenyl or N-isoindole-1,3-dione; and provided that A₂ is other than 3,5-di-t-butyl-phenyl;
W is CH or N;
Q is selected from the group consisting of (a)-(g) wherein:
   (a) —NH(CH₂)₂—Ar₁ wherein Ar₁ is pyridinyl substituted with one to three C₁₋₄alkyl substituents or a substituent selected from the group consisting of C₁₋₄alkoxy and amino;
   (b) is —NHCH(R_{z})-Ar₂ wherein R_{z} is H or C₁₋₃alkyl; Ar₂ is pyridinyl, pyrimidinyl, pyrazinyl,

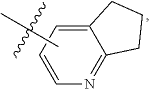

1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; and wherein Ar₂ is optionally substituted with one to three substituents independently selected from the group consisting of C₁₋₄alkyl, trifluoromethyl, hydroxyl-C₁₋₄alkyl, amino(C₁₋₄alkyl, (C₁₋₄alkyl)amino-(C₁₋₄)alkyl, di(C₁₋₄alkyl)amino-(C₁₋₄)alkyl, C₁₋₄alkoxy, C₃₋₈cycloalkylamino, amino, (C₁₋₆alkyl)amino, and di(C₁₋₆alkyl)amino; or Ar₂ is optionally substituted with one amino group and three substituents independently selected from the group consisting of C₁₋₄alkyl and C₁₋₄alkoxy;
wherein the C₁₋₆)alkyl group of (C₁₋₆alkyl)amino and di(C₁₋₆)alkyl)amino is optionally substituted with (C₁₋₄)alkyl)amino, di(C₁₋₄alkyl)amino, C₁₋₄alkoxy, C₁₋₄alkylthio, hydroxy, a 5 to 6 membered heteroaryl, or a 5 to 6 membered heterocyclyl; wherein a nitrogen atom of the 5 to 6 membered heterocyclyl is optionally substituted with a C₁₋₄alkyl substituent;
and wherein pyridin-2-yl and pyridin-3-yl are optionally further substituted with N-pyrrolidinyl, N-piperazinyl, N-piperidinyl, N-morpholinyl, N-thiomorpholinyl, —CH₂—O—CH₂—PH, and phenyl; wherein the phenyl substituent of pyridin-2-yl and pyridin-3-yl is optionally substituted with one to three substituents independently selected from the group consisting of C₁₋₄)alkyl, C₁₋₄alkoxy, and halogen;
provided that when Q is —NHCH₂(2-amino-pyridin-3-yl), and A₁ is pyridin-4-yl, 4-C₁₋₄alkyl-phenyl, or 3,4-dichloro-phenyl, A₂ is other than 4-methoxy-phenyl;
provided that when Q is —NHCH₂(2-amino-pyridin-3-yl), and A₁ is benzotriazol-1-yl, A₂ is other than 4-difluoromethoxy-phenyl;
provided that when Q is —NHCH₂(2-amino-pyridin-3-yl), L₁ is —(CH₂)₂—, and A₁ is 4-nitro-phenyl, A₂ is other than 4-methoxy-phenyl;
provided that when Q is —NHCH₂(2-amino-pyridin-3-yl), and A₁ is 4-fluoro-phenyl, A₂ is other than 4-fluoro-phenyl;
provided that when Q is —NHCH₂(6-amino-pyridin-2-yl), and A₁ is 4-fluoro-phenyl, A₂ is other than 4-trifluoromethoxy-phenyl;
provided that when Q is —NHCH₂(6-methyl-pyridin-2-yl), and A₁ is 4-methoxy-phenyl, A₂ is other than 4-methoxy-phenyl;
provided that when Q is —NHCH₂(imidazo[1,2-a]pyridinyl), and A₁ is 4-fluoro-phenyl, A₂ is other than 4-methoxy-phenyl;
provided that when Q is —NHCH₂(pyridin-4-yl), and A₁ is unsubstituted phenyl or 3,4-dichloro-phenyl, A₂ is other than 4-methoxy-phenyl;
provided that when Q is —NHCH₂(4,6-dimethyl-pyridin-3-yl), and A₁ is 4-methoxy-phenyl, —P-A₂ is other than —(CH₂)₅-methoxy;
provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, and 3-nitro-phenyl;
provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
(c) is —CH₂NHCH₂—Ar₃, wherein W is N or CH, and Ar₃ is pyridinyl optionally substituted with amino;
(d) is —(CH₂)₂—Ar₄, wherein Ar₄ is pyridinyl, or pyrimidinyl; wherein Ar₄ is optionally substituted with one to two substituents independently selected from the group consisting of C₁₋₄alkyl, C₁₋₄alkoxy, amino, (C₁₋₆alkyl)amino, and di(C₁₋₆alkyl)amino;
(e) is —CH═CH-pyridinyl;
(f) is —O—CH(R₁)—Ar₆ when W is CH; or, (f) is —S—CH(R₁)—Ar₆ and W is N or CH; wherein R₁ is hydrogen or C₁₋₄alkyl, and Ar₆ is pyridinyl or pyrimidinyl; wherein Ar₆ is optionally substituted with one to three substituents independently selected from the group consisting of C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
provided that when Q is —O—CH(R₁)—Ar₆, A₁ and A₂ are 4-methoxy-phenyl, and R₁ is hydrogen, Ar₆ is other than unsubstituted pyridin-2-yl or 2-amino-pyridin-4-yl;
and
(g) is —X₁—(CH(Rₓ))₂—Ar₇ and W is CH; wherein X₁ is O, Rₓ is H, and Ar₇ is pyridinyl or pyrimidinyl; wherein Ar₇ is optionally substituted with one to two substituents independently selected from the group consisting of C₁₋₄alkyl, C₁₋₄alkoxy, amino, (C₁₋₆alkyl)amino, and di(C₁₋₆)alkyl)amino;
provided that when Q is —O (CH₂)₂—Ar₇ and A₁ and A₂ are 4-methoxy-phenyl, Ar₇ is other than unsubstituted pyridin-2-yl or unsubstituted pyridin-3-yl;
   wherein a nitrogen atom of Ar₁, Ar₂, Ar₃, Ar₄, Ar₆, and Ar₇ is optionally substituted with oxo.

In an embodiment, the present invention is directed to a method for treating, ameliorating, or preventing pain comprising, consisting of, and/or consisting essentially of administering to a subject in need thereof, a therapeutically effective amount of a compound of Formula (I):

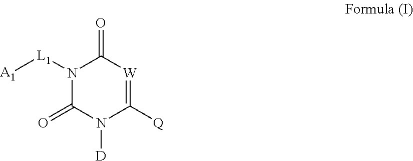

or an enantiomer, diastereomer, solvate, or pharmaceutically acceptable salt thereof;

wherein:
A₁ is aryl, heteroaryl, or a benzofused heterocyclyl selected from the group consisting of benzo[1,3]dioxalyl and 2,3-dihydro-benzofuranyl; wherein aryl and heteroaryl are optionally substituted with one to three substituents independently selected from the group consisting of C₁₋₃)alkyl, methoxy, fluoro, chloro, trifluoromethyl, trifluoromethoxy, and methylthio;
L₁ is —CH₂—;
D is —P-A₂;
wherein P is —CH₂— when A₂ is phenyl, benzofused heterocyclyl, or heteroaryl; alternatively, P is —(CH₂)₄₋₆—, when A₂ is C₁₋₄alkoxy;
A₂ is C₁₋₄alkoxy, phenyl, benzofused heterocyclyl, or a heteroaryl other than pyridin-4-yl; wherein phenyl and heteroaryl are optionally substituted with one to two substituents independently selected from the group consisting of C₁₋₄alkyl, C₁₋₄alkoxy, fluoro, chloro, halogenated C₁₋₄alkoxy, N-isoindole-1,3-dione, C₁₋₄alkylthio, C₁₋₄alkylsulfonyl, C₁₋₄alkoxycarbonyl, nitro, hydroxy, and C₁₋₄alkylcarbonylamino; provided that no more than one substituent on A₂ is N-isoindole-1,3-dione; and provided that A₂ is other than 3,5-di-t-butyl-phenyl;
W is N or CH;
Q is selected from the group consisting of (b) and (d) wherein:
   (b) is —NHCH₂—Ar₂ wherein Ar₂ is pyridinyl, pyrimidinyl, or quinolinyl; such that the point of attachment to quinolinyl is at the 2, 3, or 4-position; and wherein Ar₂ is optionally substituted with one to three substituents independently selected from the group consisting of C₁₋₄alkyl, trifluoromethyl, C₁₋₄alkoxy, amino, (C₁₋₄alkyl)amino, and di(C₁₋₄alkyl)amino;
   wherein the C₁₋₄alkyl group of (C₁₋₄alkyl)amino and di(C₁₋₄alkyl)amino is optionally substituted with (C₁₋₄alkyl)amino, di(C₁₋₄alkyl)amino, C₁₋₄alkoxy, C₁₋₄alkylthio, hydroxy, a 5 to 6 membered heteroaryl, or a 5 to 6 membered heterocyclyl;
   and wherein pyridin-2-yl and pyridin-3-yl are optionally further substituted with N-morpholinyl;
   provided that when Q is —NHCH₂(2-amino-pyridin-3-yl), and A₁ is pyridin-4-yl, 4-C₁₋₃alkyl-phenyl, or 3,4-dichloro-phenyl, A₂ is other than 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is unsubstituted phenyl or 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, and 3-nitro-phenyl;
   provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-trifluoromethyl-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
   and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
   (d) is —(CH₂)₂—Ar₄ and W is CH; wherein Ar₄ is pyridinyl is optionally substituted with one to two substituents independently selected from the group consisting of C₁₋₄alkyl, C₁₋₄alkoxy, amino, (C₁₋₆alkyl)amino, and di(C₁₋₆alkyl)amino; wherein a nitrogen atom of Ar₂ and Ar₄ is optionally substituted with oxo.

In an embodiment, the present invention is directed to a method for treating, ameliorating, or preventing pain; or a disease, syndrome, condition, or disorder that causes such pain; comprising, consisting of, and/or consisting essentially of administering to a subject in need thereof, a therapeutically effective amount of a prokineticin receptor antagonist of Formula (I):

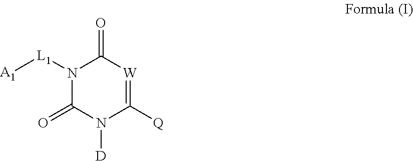

or an enantiomer, diastereomer, solvate, or pharmaceutically acceptable salt thereof;

wherein:
A₁ is substituted phenyl, heteroaryl, or a benzofused heterocyclyl selected from the group consisting of benzo[1,3]dioxalyl and 2,3-dihydro-benzofuranyl; wherein substituted phenyl is substituted with, and heteroaryl is optionally substituted with, one to three substituents independently selected from the group consisting of C₁₋₃alkyl, methoxy, fluoro and methylthio;
L₁ is —CH₂—;
D is —P-A₂; wherein P is —CH₂— when A₂ is phenyl, benzofused heterocyclyl or heteroaryl; alternatively, P is —(CH₂)₄₋₆—, when A₂ is C₁₋₄alkoxy;
A₂ is C₁₋₄alkoxy, phenyl, benzofused heterocyclyl, or a heteroaryl other than pyridin-4-yl; wherein phenyl and heteroaryl are optionally substituted with one to two substituents independently selected from the group consisting of C₁₋₄alkoxy, fluoro, halogenated C₁₋₄alkoxy, C₁₋₄alkylthio, C₁₋₄alkylsulfonyl, C₁₋₄alkoxycarbonyl, nitro, and hydroxy;
W is N or CH;
Q is selected from the group consisting of (b) and (d) wherein:
   (b) is —NHCH₂—Ar₂ wherein Ar₂ is pyridin-2-yl, pyridin-3-yl, or pyrimidinyl; wherein Ar₂ is optionally substituted with one to three substituents independently selected from the group consisting of C₁₋₄alkyl, trifluoromethyl, C₁₋₄alkoxy, amino, and (C₁₋₄alkyl)amino;
   wherein the C₁₋₄alkyl group of (C₁₋₄alkyl)amino is optionally substituted with di(C₁₋₄alkyl)amino, C₁₋₄alkoxy, or hydroxy;
   and wherein pyridin-2-yl and pyridin-3-yl are optionally further substituted with N-morpholinyl;
   provided that when Q is —NHCH₂(2-amino-pyridin-3-yl), and A₁ is pyridin-4-yl, 4-C₁₋₃alkyl-phenyl or 3,4-dichloro-phenyl, A₂ is other than 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(pyridin-4-yl), and A₁ is 3,4-dichloro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl) and A₁ is 4-methoxy-phenyl, provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2,4-difluoro-phenyl, 2,6-difluoro-phenyl, 2,6-dichloro-phenyl, 2-chloro-4-fluoro-phenyl, or 2,6-difluoro-4-methoxy-phenyl, A₂ is other than 4-difluoromethoxy-phenyl;
   and, provided that when Q is —NHCH₂(4,6-dimethyl-pyridin-3-yl) and A₁ is 2,6-difluoro-4-methoxy-phenyl or 3,4-dichloro-phenyl, A₂ is other than 4-methoxy-phenyl;
   (d) is —(CH₂)₂—Ar₄ and W is CH; wherein Ar₄ is pyridinyl is optionally substituted with amino;
      wherein a nitrogen atom of Ar₂ and Ar₄ is optionally substituted with oxo.

In an embodiment, the present invention is directed to a method for treating, ameliorating, or preventing pain comprising, consisting of, and/or consisting essentially of administering to a subject in need thereof, a therapeutically effective amount of a compound of Formula (I):

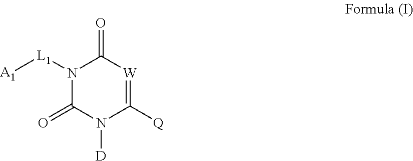

or an enantiomer, diastereomer, solvate, or pharmaceutically acceptable salt thereof;

wherein:
A₁ is substituted phenyl, benzotriazolyl, benzofuranyl, benzo[1,3]dioxalyl or 2,3-dihydro-benzofuranyl; wherein phenyl is substituted at the 4-position with methoxy, fluoro, or methylthio; and wherein A₁ other than substituted phenyl is optionally substituted with one to two substituents independently selected from the group consisting of methyl, methoxy, fluoro and methylthio;
L₁ is —CH₂—;
D is —P-A₂;
wherein P is —CH₂— when A₂ is phenyl, 2,3-dihydro-benzofuranyl, indolyl, benzofuranyl, pyridin-3-yl, or benzothiophenyl; alternatively, P is —(CH₂)₄₋₆—, when A₂ is C₁₋₄alkoxy;
A₂ is C₁₋₄alkoxy, phenyl, 2,3-dihydro-benzofuranyl, indolyl, benzofuranyl, pyridin-3-yl, or benzothiophenyl; wherein A₂ other than C₁₋₄alkoxy is optionally substituted with one to two substituents independently selected from the group consisting of C₁₋₄alkoxy, fluoro, fluorinated C₁₋₄alkoxy, C₁₋₄alkylthio, C₁₋₄alkylsulfonyl, C₁₋₄alkoxycarbonyl, nitro, and hydroxy;
W is N or CH;
Q is —NHCH₂—Ar₂ wherein Ar₂ is unsubstituted pyridin-2-yl, 4,6-dimethyl-pyridin-3-yl, 2-amino-pyridin-3-yl, or 2-((C₁₋₄alkyl)amino)-pyridin-3-yl;
   wherein the C₁₋₄alkyl group of (C₁₋₄alkyl)amino is optionally substituted with di(C₁₋₄alkyl)amino, C₁₋₄alkoxy, or hydroxy;
   and wherein 2-amino-pyridin-3-yl is optionally further substituted with 4,6-dimethyl or 4-methoxy;
   provided that when Q is —NHCH₂(2-amino-pyridin-3-yl), and A₁ is pyridin-4-yl or 4-methyl-phenyl, A₂ is other than 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl) and A₁ is 4-methoxy-phenyl, A₂ is other than 3-methoxy-phenyl or 3-nitro-phenyl;
   and
   provided that when Q is —NHCH₂(4,6-dimethyl-pyridin-3-yl) and A₁ is benzotriazol-1-yl, A₂ is other than 4-difluoromethoxy-phenyl;
      wherein a nitrogen atom of Ar₂ and Ar₄ is optionally substituted with oxo.

In an embodiment, the present invention is directed to a method for treating, ameliorating, or preventing pain comprising, consisting of, and/or consisting essentially of administering to a subject in need thereof, a therapeutically effective amount of a compound of Formula (I):

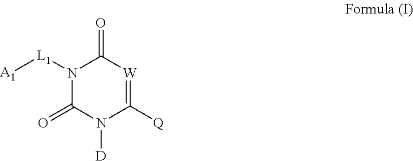

or an enantiomer, diastereomer, solvate, or pharmaceutically acceptable salt thereof;

wherein:
A₁ is substituted phenyl, benzotriazolyl, benzofuranyl, benzo[1,3]dioxalyl or 2,3-dihydro-benzofuranyl; wherein phenyl is substituted at the 4-position with methoxy, fluoro, or methylthio; and wherein A₁ other than substituted phenyl is optionally substituted with one to two substituents independently selected from the group consisting of methyl, methoxy, fluoro and methylthio;
L₁ is —CH₂—;
D is —P-A₂;
wherein P is —CH₂— when A₂ is phenyl, 2,3-dihydro-benzofuranyl, indolyl, benzofuranyl, pyridin-3-yl, or benzothiophenyl; alternatively, P is —(CH₂)₄₋₆—, when A₂ is C₁₋₄alkoxy;
A₂ is C₁₋₄alkoxy, phenyl, 2,3-dihydro-benzofuranyl, indolyl, benzofuranyl, pyridin-3-yl, or benzothiophenyl; wherein A₂ other than C₁₋₄alkoxy is optionally substituted with one to two substituents independently selected from the group consisting of C₁₋₄alkoxy, fluoro, fluorinated C₁₋₄alkoxy, C₁₋₄alkylthio, C₁₋₄alkylsulfonyl, C₁₋₄alkoxycarbonyl, nitro, and hydroxy;
W is N;
Q is —NHCH₂—Ar₂ wherein Ar₂ is unsubstituted pyridin-2-yl, 4,6-dimethyl-pyridin-3-yl, 2-amino-pyridin-3-yl, or 2-((C₁₋₄alkyl)amino)-pyridin-3-yl;
   wherein the C₁₋₄alkyl group of (C₁₋₄alkyl)amino is optionally substituted with di(C₁₋₄alkyl)amino, C₁₋₄alkoxy, or hydroxy;
   and wherein 2-amino-pyridin-3-yl is optionally further substituted with 4,6-dimethyl or 4-methoxy;
   provided that when Q is —NHCH₂(2-amino-pyridin-3-yl), and A₁ is pyridin-4-yl or 4-methyl-phenyl, A₂ is other than 4-methoxy-phenyl;
   provided that when Q is —NHCH₂(2-amino-pyridin-3-yl), and A₁ is benzotriazol-1-yl, A₂ is other than 4-difluoromethoxy-phenyl;
   provided that when Q is —NHCH₂(2-amino-pyridin-3-yl), and A₁ is 4-fluoro-phenyl, A₂ is other than 4-fluoro-phenyl;
   provided that when Q is —NHCH₂(6-amino-pyridin-2-yl), and A₁ is 4-fluoro-phenyl, A₂ is other than 4-trifluoromethoxy-phenyl;
   provided that when Q is —NHCH₂(6-methyl-pyridin-2-yl), and A₁ is 4-methoxy-phenyl, A₂ is other than 4-methoxy-phenyl;
   provided that when Q is —NHCH₂(imidazo[1,2-a]pyridinyl), and A₁ is 4-fluoro-phenyl, A₂ is other than 4-methoxy-phenyl;
   provided that when Q is —NHCH₂(4,6-dimethyl-pyridin-3-yl), and A₁ is 4-methoxy-phenyl, —P-A₂ is other than —(CH₂)₅-methoxy;
   provided that when Q is —NHCH₂(4,6-dimethyl-pyridin-3-yl) and A₁ is 4-methoxy-phenyl, A₂ is other than 3-methoxy-phenyl or 3-nitro-phenyl;
   and
   provided that when Q is —NHCH₂(4,6-dimethyl-pyridin-3-yl) and A₁ is benzotriazol-1-yl, A₂ is other than 4-difluoromethoxy-phenyl;
      wherein a nitrogen atom of Ar₂ and Ar₄ is optionally substituted with oxo.

In an embodiment, the present invention is directed to a method for treating, ameliorating, or preventing pain comprising, consisting of, and/or consisting essentially of administering to a subject in need thereof, a therapeutically effective amount of a compound of Formula (I):

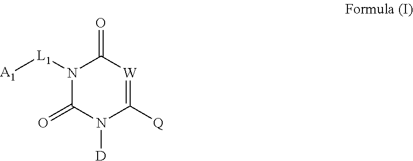

or pharmaceutically acceptable salt thereof;
selected from the group consisting of
a compound of Formula (I) wherein A₁ is 4-methoxy-phenyl, L₁ is CH₂, D is 4-methoxy-phenylmethyl, W is N, and Q is

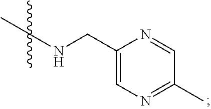

a compound of Formula (I) wherein A₁ is 4-methoxy-phenyl, L₁ is CH₂, D is 4-methoxy-phenylmethyl, W is N, and Q is

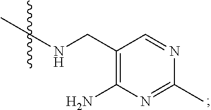

a compound of Formula (I) wherein A₁ is 4-chloro-phenyl, L₁ is CH₂, D is —(CH₂)₅OCH₃, W is N, and Q is

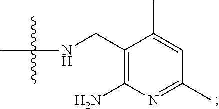

a compound of Formula (I) wherein A₁ is 3,4-dichloro-phenyl, L₁ is CH₂, D is 4-methoxy-phenylmethyl, W is N, and Q is 2-(pyridin-2-yl)ethyl-amino;
a compound of Formula (I) wherein A₁ is 3,4-dichloro-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 5-amino-pyridin-2-ylmethyl-amino;
a compound of Formula (I) wherein A₁ is 4-chloro-phenyl, L₁ is CH₂, D is 4-methoxy-phenylmethyl, W is N, and Q is 6-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-amino-pyrimidin-5-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-aminomethyl;
a compound of Formula (I) wherein A₁ is 4-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-quinolin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-pyridin-3-yl)-ethylamino;
a compound of Formula (I) wherein A₁ is 4-fluoro-phenyl, L₁ is CH₂, D is 4-methoxy-phenylmethyl, W is N, and Q is 2-N-pyrrolidinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperazinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperidinyl-pyridin-3-ylmethyl-amino;
a compound of Formula (I) wherein A₁ is 4-fluoro-phenyl, L₁ is CH₂, D is 4-methoxy-phenylmethyl, W is N, and Q is 2-methylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-butylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-thiomorpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-ethylamino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 1,2,3,4-tetrahydro-[1,8]naphthyridin-7-ylmethyl-amino;
a compound of Formula (I) wherein A₁ is 4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-2-yl, L₁ is CH₂, D is 4-methoxy-phenylmethyl, W is N, and Q is 2-amino-pyridin-3-ylmethyl-amino;
a compound of Formula (I) wherein A₁ is 4-methylthio-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-(4-fluoro-phenyl)-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-hydroxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-cyclohexylamino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-2-amino-pyridin-3-ylmethyl-amino;
a compound of Formula (I) wherein A₁ is 4-methoxy-phenyl, L₁ is CH₂, D is 4-hydroxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methylcarbonylamino-phenylmethyl, W is N, and Q is 2-amino-pyridin-3-ylmethyl-amino;
a compound of Formula (I) wherein A₁ is 4-methoxy-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-2-ylmethyl-amino;
a compound of Formula (I) wherein A₁ is 4-methoxy-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-4-ylmethyl-amino;
a compound of Formula (I) wherein A₁ is 3-methoxy-phenyl, L₁ is CH₂, D is 4-methoxy-phenylmethyl, W is N, and Q is 2-amino-pyridin-3-ylmethyl-amino;
a compound of Formula (I) wherein A₁ is phenyl, L₁ is CH₂, D is 4-methoxy-phenylmethyl, W is N, and Q is 2-amino-pyridin-3-ylmethyl-amino;
a compound of Formula (I) wherein A₁ is 4-cyano-phenyl, L₁ is CH₂, D is 4-methoxy-phenylmethyl, W is N, and Q is 2-amino-pyridin-3-ylmethyl-amino;
a compound of Formula (I) wherein A₁ is 4-trifluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-ethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-nitro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH(allyl), D is 4-methoxy-phenylmethyl, W is N, and Q is 2-amino-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-methoxy-phenyl, L₁ is CH₂, D is 4-aminocarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-pyridin-3-ylmethyl-amino;
a compound of Formula (I) wherein A₁ is 4-hydroxy-phenyl, L₁ is CH₂, D is 4-methoxy-phenylmethyl, W is N, and Q is 2-amino-pyridin-3-ylmethyl-amino;
a compound of Formula (I) wherein A₁ is 3-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-5-phenyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4-methoxy-pyridin-3-ylmethyl-amino;
a compound of Formula (I) wherein A₁ is 4-methoxy-phenyl, L₁ is CH₂, D is 4-methoxy-phenylmethyl, W is N, and Q is 6-methyl-pyridin-3-ylmethyl-amino;
a compound of Formula (I) wherein A₁ is 4-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-2-ylmethyl-amino;
a compound of Formula (I) wherein A₁ is 4-methoxy-phenyl, L₁ is CH₂, D is 4-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-trifluoromethyl-pyridin-2-ylmethyl-amino;
a compound of Formula (I) wherein A₁ is 4-methoxy-phenyl, L₁ is CH₂, D is 4-methoxy-phenylmethyl, W is N, and Q is 3-methyl-pyridin-2-ylmethyl-amino;
a compound of Formula (I) wherein A₁ is 4-methoxy-phenyl, L₁ is CH₂, D is 4-methoxy-phenylmethyl, W is N, and Q is 2-(2-methylthio-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(3-methyl-butylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(tetrahydro-furan-2-ylmethyl)-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(furan-2-ylmethyl-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-ethyl-pyrrolidin-2-ylmethyl-amino)-pyridin-3-ylmethyl-amino;
a compound of Formula (I) wherein A₁ is phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is phenoxy, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-nitro-phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methylthio-phenylmethyl, W is N, and Q is 2-amino-pyridin-3-ylmethyl-amino;
a compound of Formula (I) wherein A₁ is 4-methoxy-phenyl, L₁ is CH₂, D is pyridin-4-ylmethyl, W is N, and Q is 2-amino-pyridin-3-ylmethyl-amino;
a compound of Formula (I) wherein A₁ is 4-methoxy-phenyl, L₁ is CH₂, D is benzofuran-2-ylmethyl, W is N, and Q is 2-amino-pyridin-3-ylmethyl-amino;
a compound of Formula (I) wherein A₁ is 4-methoxy-phenyl, L₁ is CH₂, D is 5-methoxy-n-pentyl, W is N, and Q is 2-amino-pyridin-3-ylmethyl-amino;
a compound of Formula (I) wherein A₁ is 4-methoxy-phenyl, L₁ is CH₂, D is n-hexyl, W is N, and Q is 2-amino-pyridin-3-ylmethyl-amino;
a compound of Formula (I) wherein A₁ is 4-methoxy-phenyl, L₁ is CH₂, D is 3-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-cyano-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-cyano-phenylmethyl, W is N, and Q is 2-amino-pyridin-3-ylmethyl-amino;
a compound of Formula (I) wherein A₁ is 4-iodo-phenyl, L₁ is CH₂, D is 4-methoxy-phenylmethyl, W is N, and Q is 2-amino-pyridin-3-ylmethyl-amino;
a compound of Formula (I) wherein A₁ is 4-pyrazol-1-yl-phenyl, L₁ is CH₂, D is 4-methoxy-phenylmethyl, W is N, and Q is 2-amino-pyridin-3-ylmethyl-amino;
a compound of Formula (I) wherein A₁ is 4-fluoro-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 2-(4-methoxy-phenyl)-ethyl, W is N, and Q is 2-amino-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 2-amino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methylthio-phenylmethyl, W is N, and Q is 4,6-dimethyl-pyridin-3-ylmethyl-amino;
a compound of Formula (I) wherein A₁ is 4-methoxy-phenyl, L₁ is CH₂, D is pyridin-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-2-ylmethyl, W is N, and Q is 4,6-dimethyl-pyridin-3-ylmethyl-amino;
a compound of Formula (I) wherein A₁ is 4-methoxy-phenyl, L₁ is CH₂, D is n-hexyl, W is N, and Q is 4,6-dimethyl-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 4,6-dimethyl-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-methoxy-phenylmethyl, W is N, and Q is 4,6-dimethyl-pyridin-3-ylmethyl-amino;
a compound of Formula (I) wherein A₁ is 4-ethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-nitro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH(allyl), D is 4-methoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is pyridin-4-ylmethyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-amino-pyridin-2-ylmethyl-amino;
a compound of Formula (I) wherein A₁ is 4-methoxy-phenyl, L₁ is CH₂, D is 4-fluoro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-chloro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-4,6-dimethyl-pyridin-3-ylmethyl-amino;
a compound of Formula (I) wherein A₁ is indol-3-yl, L₁ is CH₂CH₂, D is 4-methoxy-phenylmethyl, W is N, and Q is 2-amino-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethoxy;
a compound of Formula (I) wherein A₁ is 4-methoxy-phenyl, L₁ is CH₂, D is 4-methoxy-phenylmethyl, W is N, and Q is 6-trifluoromethyl-pyridin-3-ylmethyl-amino;
a compound of Formula (I) wherein A₁ is 2,3-dihydro-benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-nitro-4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is indol-5-ylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is indol-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-t-butoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzothiophen-5-ylmethyl, W is N, and Q is 2-amino-pyridin-3-ylmethyl-amino;
a compound of Formula (I) wherein A₁ is 4-fluoro-phenoxy, L₁ is CH₂CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is benzothiophen-5-ylmethyl, W is N, and Q is 4,6-dimethyl-pyridin-3-ylmethyl-amino;
a compound of Formula (I) wherein A₁ is 2-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 2-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzothiophen-5-yl, L₁ is CH₂, D is 4-methoxy-phenylmethyl, W is N, and Q is 2-amino-pyridin-3-ylmethyl-amino;
a compound of Formula (I) wherein A₁ is benzothiophen-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-n-propylamino-pyridin-2-ylmethyl-amino;
a compound of Formula (I) wherein A₁ is 4-methoxy-phenyl, L₁ is CH₂, D is 4-methoxy-phenylmethyl, W is CH, and Q is 6-amino-pyridin-2-ylmethyl-amino;
a compound of Formula (I) wherein A₁ is 4-methoxy-phenyl, L₁ is CH₂, D is 4-methoxy-cyclohexylmethyl, W is N, and Q is 2-amino-pyridin-3-ylmethyl-amino;
a compound of Formula (I) wherein A₁ is 4-methoxy-phenyl, L₁ is CH₂, D is 4-methoxy-cyclohexylmethyl, W is N, and Q is 4,6-dimethyl-pyridin-3-ylmethyl-amino;
a compound of Formula (I) wherein A₁ is 4-methoxy-phenyl, L₁ is CH₂, D is 3,4-dichloro-phenylmethyl, W is N, and Q is 2-amino-pyridin-3-ylmethyl-amino;
a compound of Formula (I) wherein A₁ is 4-methoxy-phenyl, L₁ is CH₂, D is 4-(isoindol-1,3-dione-2-yl)-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-methoxycarbonyl-n-propyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-pyridin-2-yl-ethylamino;
a compound of Formula (I) wherein A₁ is 4-methoxy-phenyl, L₁ is CH₂, D is indol-4-ylmethyl, W is N, and Q is 2-amino-4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 6-amino-pyridin-2-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-pyrazol-1-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-iodo-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-[1,2,3]thiadiazol-4-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-3-yl-ethyl;
a compound of Formula (I) wherein A₁ is 4-methoxy-phenyl, L₁ is CH₂, D is indol-6-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzothiophen-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-hydroxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methylcarbonyloxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenyl, W is CH, and Q is 2-pyridin-4-yl-ethyl;
a compound of Formula (I) wherein A₁ is 4-methoxy-phenyl, L₁ is CH₂, D is 4-methoxy-phenyl, W is CH, and Q is cis-2-pyridin-4-yl-vinyl;
a compound of Formula (I) wherein A₁ is 2,3-dihydro-benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-2-yl-ethyl;
a compound of Formula (I) wherein A₁ is 4-methoxy-phenyl, L₁ is CH₂, D is 4-methoxy-phenylmethyl, W is N, and Q is imidazo[1,2-a]pyridin-8-ylmethyl-amino;
a compound of Formula (I) wherein A₁ is 4-methoxy-phenyl, L₁ is CH₂, D is 4-methoxy-phenylmethyl, W is CH, and Q is 2-(2-aminocarbonyl-pyridin-3-yl)-ethyl;
a compound of Formula (I) wherein A₁ is 4-methoxy-phenyl, L₁ is CH₂, D is 4-methoxy-phenylmethyl, W is CH, and Q is 2-amino-pyridin-3-ylmethoxy;
a compound of Formula (I) wherein A₁ is 4-hydroxymethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-aminocarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,6-difluoro-4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-methoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(2-amino-pyridin-3-yl)-ethyl;
a compound of Formula (I) wherein A₁ is 4-methoxy-phenyl, L₁ is CH₂, D is 2,4-dimethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-4,6-dimethyl-pyridin-3-ylmethoxy;
a compound of Formula (I) wherein A₁ is 4-methoxy-phenyl, L₁ is CH₂, D is 3-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-fluoro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-methoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethylthio;
a compound of Formula (I) wherein A₁ is 4-methoxy-phenyl, L₁ is CH₂, D is 2-methyl-2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-piperidinyl)-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(4-amino-pyridin-3-yl)-ethyl;
a compound of Formula (I) wherein A₁ is 4-methoxy-phenyl, L₁ is CH₂, D is 4-methoxy-phenylmethyl, W is N, and Q is 2-(pyridin-4-yl)-ethylamino;
a compound of Formula (I) wherein A₁ is 1-methyl-1H-benzotriazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(6-amino-pyridin-2-yl)ethyl;
a compound of Formula (I) wherein A₁ is 4-methoxy-phenyl, L₁ is CH₂, D is 5-methoxy-n-pentyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 1-(2-amino-pyridin-4-yl)-ethoxy;
a compound of Formula (I) wherein A₁ is 2,3-dihydro-benzofuran-5-yl, L₁ is CH₂, D is 2,3-dihydro-benzofuran-5-ylmethyl, W is N, and Q is N-oxo-2-amino-4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 2-amino-pyridin-3-ylmethyl-amino;
a compound of Formula (I) wherein A₁ is indol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyrimidin-4-ylmethoxy;
   and
a compound of Formula (I) wherein A₁ is 2,3-dihydro-benzofuran-5-yl, L₁ is CH₂, D is 4-difluoromethoxy-phenylmethyl, W is N, and Q is N-oxo-2-amino-4,6-dimethyl-pyridin-3-ylmethyl-amino;
   and combinations thereof.

For use in medicine, salts of compounds of Formula (I) refer to non-toxic “pharmaceutically acceptable salts.” Other salts may, however, be useful in the preparation of compounds of Formula (I) or of their pharmaceutically acceptable salts thereof. Suitable pharmaceutically acceptable salts of compounds of Formula (I) include acid addition salts which can, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts, such as sodium or potassium salts; alkaline earth metal salts, such as calcium or magnesium salts; and salts formed with suitable organic ligands, such as quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases that may be used in the preparation of pharmaceutically acceptable salts include acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholin, piperazine, potassium hydroxide, 1-(2-hydroxyethyl)-pyrrolidine, secondary amine, sodium hydroxide, triethanolamine, tromethamine and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds according to embodiments of this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula I.

Where the processes for the preparation of the compounds according to certain embodiments of the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

One embodiment of the present invention is directed to a composition, including a pharmaceutical composition, comprising, consisting of, and/or consisting essentially of the (+)-enantiomer of a compound of Formula (I) wherein said composition is substantially free from the (−)-isomer of said compound. In the present context, substantially free means less than about 25%, preferably less than about 10%, more preferably less than about 5%, even more preferably less than about 2% and even more preferably less than about 1% of the (−)-isomer calculated as.

$$\% ( + ) \text{-} enantiomer = \frac{( mass ( + ) \text{-} enantiomer )}{( mass ( + ) \text{-} enantiomer ) + ( mass ( - ) \text{-} enantiomer )} \times 100.$$

Another embodiment of the present invention is a composition, including a pharmaceutical composition, comprising, consisting of, and consisting essentially of the (−)-enantiomer of a compound of Formula (I) wherein said composition is substantially free from the (+)-isomer of said compound. In the present context, substantially free from means less than about 25%, preferably less than about 10%, more preferably less than about 5%, even more preferably less than about 2% and even more preferably less than about 1% of the (+)-isomer calculated as

$$\% ( - ) \text{-} enantiomer = \frac{( mass ( - ) \text{-} enantiomer )}{( mass ( + ) \text{-} enantiomer ) + ( mass ( - ) \text{-} enantiomer )} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups, such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent

By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

Solid oral dosage forms, such as tablets or capsules, containing the compounds of the present invention may be administered in at least one dosage form at a time, as appropriate. It is also possible to administer the compounds in sustained release formulations.

Additional oral forms in which the present inventive compounds may be administered include exilirs, solutions, syrups, and suspensions; each optionally containing flavoring agents and coloring agents.

Alternatively, compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a white wax or white soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example intracavernosally, intravenously, intramuscularly, subcutaneously, intradermally or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus for liquid oral preparations, such as suspensions, syrups, elixirs and solutions, suitable carriers, excipients and diluents include water, glycols, oils, alcohols, flavoring agents, preservatives, stabilizers, coloring agents and the like; for solid oral preparations, such as powders, capsules and tablets, suitable carriers, excipients and diluents include starches, sugars, diluents, granulating agents, lubricants, binders, disintegrating agents and the like. Solid oral preparations also may be optionally coated with substances, such as, sugars, or be enterically-coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives, such as solubilizers and preservatives.

A therapeutically effective amount of a compound of Formula (I) or a pharmaceutical composition thereof includes a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about 4 times per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition is preferably provided in the form of tablets containing about 0.01, about 10, about 50, about 100, about 150, about 200, about 250, and about 500 milligrams of a compound of Formula (I).

Advantageously, a compound of Formula (I) may be administered in a single daily dose, or the total daily dosage may be administered in divided doses of two, three and four times daily.

Optimal dosages of a compound of Formula (I) to be administered may be readily determined and will vary with the particular compound used, the mode of administration, the strength of the preparation and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is required for a subject in need thereof.

As Prokineticin 1 receptor antagonists, the compounds of Formula (I) are useful in methods for treating, ameliorating, or preventing pain in a subject, including an animal, a mammal and a human. Such methods comprise, consist of and/or consist essentially of administering to a subject, including an animal, a mammal, and a human in need of such treatment or prevention a therapeutically effective amount of a compound, salt or solvate of Formula (I).

Representative IUPAC names for the compounds of the present invention were derived using the ACD/LABS SOFTWARE™ Index Name Pro Version 4.5 nomenclature software program provided by Advanced Chemistry Development, Inc., Toronto, Ontario, Canada.

Abbreviations used in the instant specification, particularly the Schemes and Examples, are as follows
AIBN=2,2′-azobisisobutyronitrile
Boc=tert-butoxycarbonyl
BuLi=n-butyllithium
Cpd or Cmpd=compound
d=day/days
DCM=dichloromethane
DIAD=diisopropyl azodicarboxylate
DIPEA or DIEA=diisopropylethylamine
DMEM=Dulbecco's Modified Eagle Medium
DMF=N,N-dimethylformamide
DMSO=dimethylsulfoxide
EDCI=1-(3-Dimethylaminopropyl)-3-ethylcarbodiimide hydrochloride
EtOAc=ethyl acetate
EtOH=ethanol
h=hour/hours
HBTU=O-Benzotriazol-1-yl-N,N,N′,N′-tetramethyluronium hexafluorophosphate
LDA=lithium diisopropylamide
M=molar
MeCN=acetonitrile
MeOH=methanol
min=minutes
NaOMe=sodium methoxide
NBS=N-bromosuccinimide
PyBOP=benzotriazole-1-yl-oxy-tris-pyrrolidino-phosphonium hexafluorophosphate
rt/RT=room temperature
TBAF=tetra-n-butylammonium fluoride
TEBA=benzyltriethylammonium chloride
THF=tetrahydrofuran
TFA=trifluoroacetic acid
UHP=urea-hydrogen peroxide addition complex
μw=microwave

### General Schemes

Representative compounds of the present invention can be synthesized in accordance with the general synthetic methods described below and are illustrated in the schemes that follow. The starting materials and reagents used in the schemes that follow are understood to be either commercially available or prepared by methods known to those skilled in the art. Since the schemes are an illustration, the invention should not be construed as being limited by the chemical reactions and conditions expressed.

Scheme A describes the preparation of certain compounds of the present invention wherein Q of Formula (I) is (a) or (b) and W is N. More specifically, Q is —NH(CH₂)₂Ar₁ or —NHCH(R_{z})-Ar₂. In Scheme A, n is 1 or 2 and Arₘ is Ar₁ or Ar₂, such that when n is 2, Arₘ is Ar₁, and when n is 1 and R_{z} is H or C₁₋₃alkyl, Arₘ is Ar₂.

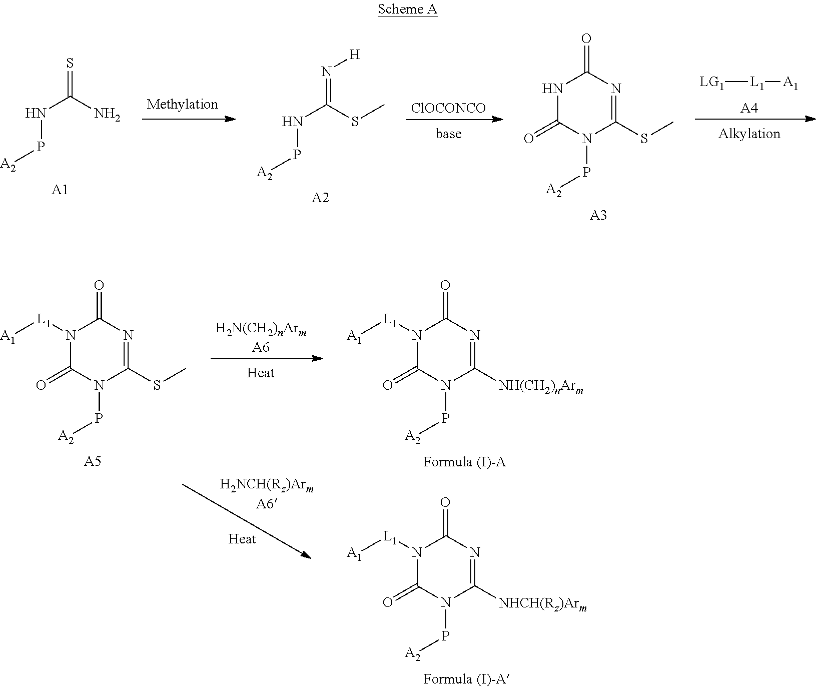

A compound of formula A1 is either commercially available or may be prepared by known methods described in the scientific literature. A compound of formula A1 may be methylated with a methylating agent such as methyl iodide in a polar solvent such as methanol to give a compound of formula A2. A compound of formula A2 may be condensed with an appropriately substituted isocyanate such as N-chlorocarbonyl isocyanate in the presence of excess of a tertiary amine such as diisopropylethylamine to give a triazine of formula A3. A compound of formula A3 may be alkylated with a compound of formula A4, which is either commercially available or may be prepared by known methods described in the scientific literature, wherein LG₁ is a leaving group, using conventional chemistry known to one versed in the art. For instance, when LG₁ is a hydroxy group, compound A4 may be coupled with a compound of formula A3 in the presence of a coupling agent such as DIAD in a non-alcoholic polar solvent such as THF or methylene chloride. Alternatively, LG₁ may be a halide, tosylate, or the like such that LG₁ is displaced by the amino portion of a compound of A3 to give a compound of formula A5. The Q-portion of a compound of Formula (I)-A may be installed by treating a compound of formula A5 with a compound of formula A6 or A6′ to afford a compound of Formula (I)-A or (I)-A′, respectively.

Scheme A-1 describes the synthesis of intermediates of formula A6.

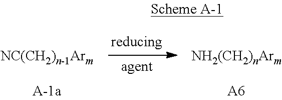

A compound of formula A-1a is either commercially available or may be prepared by known methods described in the scientific literature. A compound of formula A-1a may be reduced under various reaction conditions, such as Raney Nickel with hydrazine or under a pressurized atmosphere of hydrogen gas in the presence of an organometallic catalyst such as Pd/C, to afford a compound of formula A6.

Scheme B illustrates the general synthesis of compounds of the present invention wherein Q of Formula (I) is (d) or (e) and W is N. More specifically, Q is —(CH₂)₂Ar₄ or —CH═CH—Ar₅. In Scheme B, Ar_{y} is Ar₄ or Ar₅.

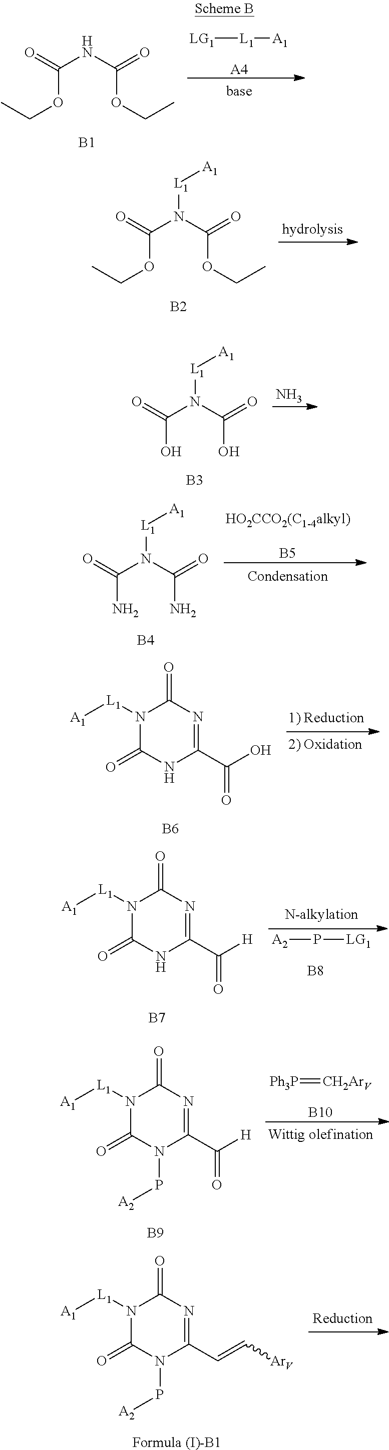

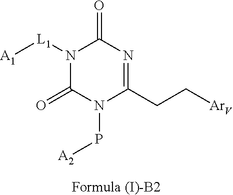

A compound of formula B1 (either commercially available or prepared by known methods described in the scientific literature) may be treated with a base followed by alkylation with a compound of formula A4 to afford a compound of formula B2. Treatment of a compound of formula B2 with an aqueous base such as hydroxide gives a compound of formula B3, which upon treatment with ammonia or its equivalent provides a compound of formula B4. The compound of formula B4 may then be condensed with a compound of formula B5 to form a triazine compound of formula B6.

Using conventional reagents and methods known to one of ordinary skill in the art, the carboxy group of a compound of formula B6 may be reduced to its corresponding alcohol, followed by oxidation to an aldehyde of formula B7. The secondary amino group of the triazinyl ring may be alkylated with a compound of formula B8 using coupling chemistry or standard alkylation chemistry to afford a compound of formula B9. The aldehyde portion of the compound may participate in a Wittig olefination with a compound of formula B10 to provide a compound of formula Formula (I)-B1. The compound of Formula (I)-B1 can be reduced under standard hydrogenation conditions to afford a compound of Formula (I)-B2.

Scheme C illustrates the general synthesis of compounds of the present invention wherein Q of Formula (I) is (d) or (e) and W is C(R_{W}).

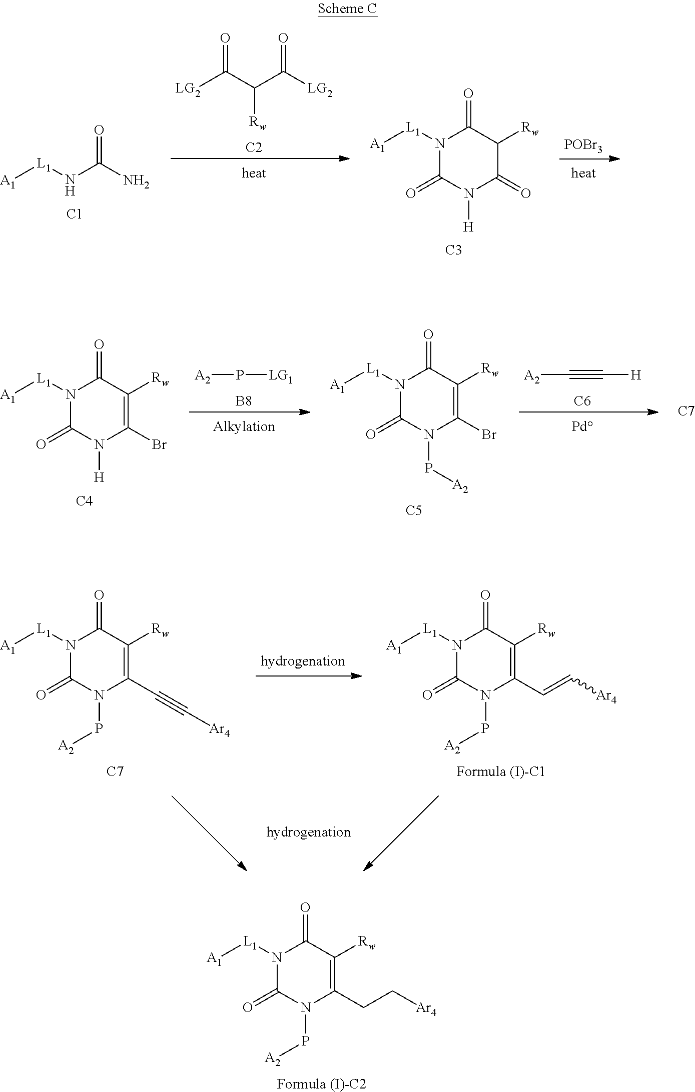

A compound of formula C1 (either commercially available or prepared by known methods described in the scientific literature) may be condensed with a compound of formula C2 with heating, wherein LG₂ is C₁₋₄alkoxy, choro, or the like, to form a compound of formula C3. Compound C3 can be reacted with phosphorus oxybromide with heating to provide a bromo-uracil of formula C4. A compound of formula C4 may be alkylated with a compound of formula B8 to provide a compound of formula C5. A compound of formula C5 may be coupled with a compound of formula C6 in the presence of an organometallic reagent such as tetrakis(triphenylphosphine)-palladium to yield a compound of formula C7. Hydrogenation of a compound of formula C7 provides a compound of formula Formula (I)-C1 which may be further reduced by prolonged exposure to hydrogenation conditions to yield a compound of Formula (I)-C2. Alternatively, a compound of formula C7 may be converted directly to a compound of Formula (I)-C2 using conventional hydrogenation reagents and methods. One of ordinary skill in the art will recognize that the duration of exposure of a compound to hydrogenation conditions is one way of controlling the degree of reduction of an alkyne to an alkene or alkane.

Scheme D illustrates the general synthesis of compounds of the present invention wherein Q of Formula (I) is (a) or (b) and W is C(R_{W}). Scheme D also illustrates the general synthesis of compounds of the present invention wherein Q if Formula (I) is (g) and W is C(R_{W}).

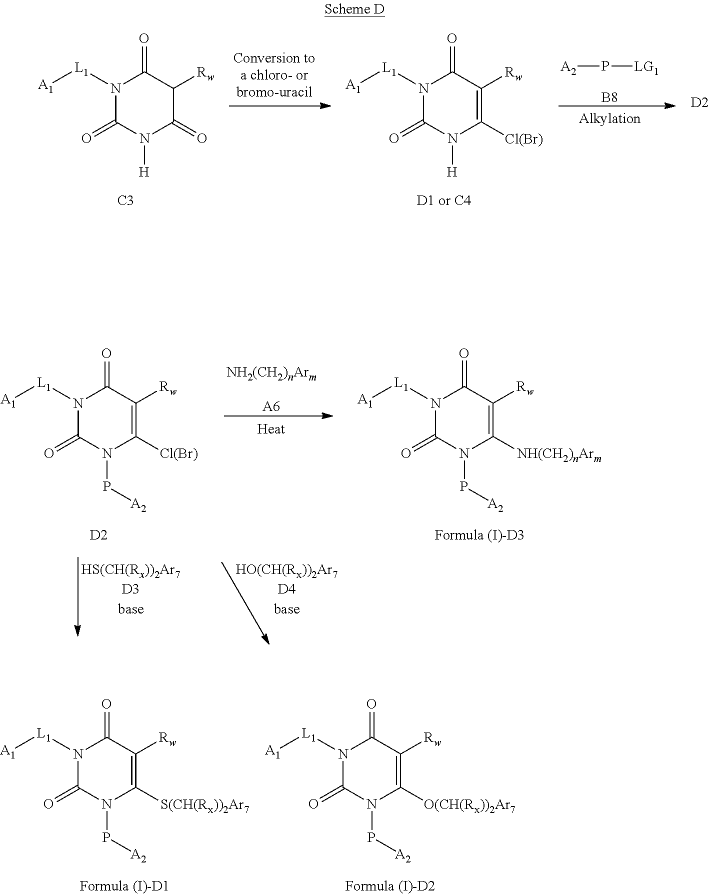

A compound of formula C3 may be treated with phosphorus oxychloride, PCl₅, or the like, with heating to afford a compound of formula D1; alternatively, the bromo analog (Formula C4) may be used in this synthetic sequence. A compound of formula B8 may be used to install —P-A₂ via conventional alkylation procedures as described herein. A compound of formula D2 may be elaborated via a nucleophilic displacement of the chloride (or bromide) with an amine of formula A6 (wherein Arₘ is defined as Ar₁ or Ar₂) to afford a compound of Formula (I)-D3. A compound of formula D2 may be elaborated via a nucleophilic displacement of the chloride (or bromide) under basic conditions with alcohol D4 to provide a compound of Formula (I)-D2 (when X₁=O). A compound of formula D2 may also be elaborated via a nucleophilic displacement of the chloride (or bromide) under basic conditions with a compound of formula D3 to provide a compound of Formula (I)-D1 (when X₁=S).

Scheme E depicts the general synthesis of compounds of the present invention wherein Q of Formula (I) is —S—CH(R₁)Ar₆ of (f) or Q is —S(CH(Rₓ))₂—Ar₇ of (g), and W is N.

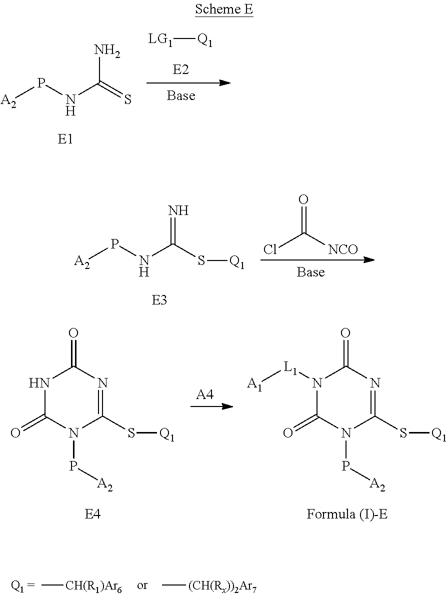

A compound of formula E1 (either commercially available or prepared by known methods described in the scientific literature) may be alkylated under basic conditions with a compound of formula E2 (wherein Q₁ is —CH(R₁)Ar₆ or —(CH(Rₓ))₂Ar₇) to provide a compound of formula E3. A compound of formula E3 may be condensed with an appropriately substituted isocyanate such as N-chlorocarbonyl isocyanate in the presence of excess tertiary amine such as diisopropylethylamine to give a triazine of formula E4. A compound of formula E4 may be alkylated with a compound of formula A4 to provide a compound of Formula (I)-E.

Scheme F illustrates the general synthesis of compounds of the present invention wherein Q of Formula (I) is (c) and W is CH.

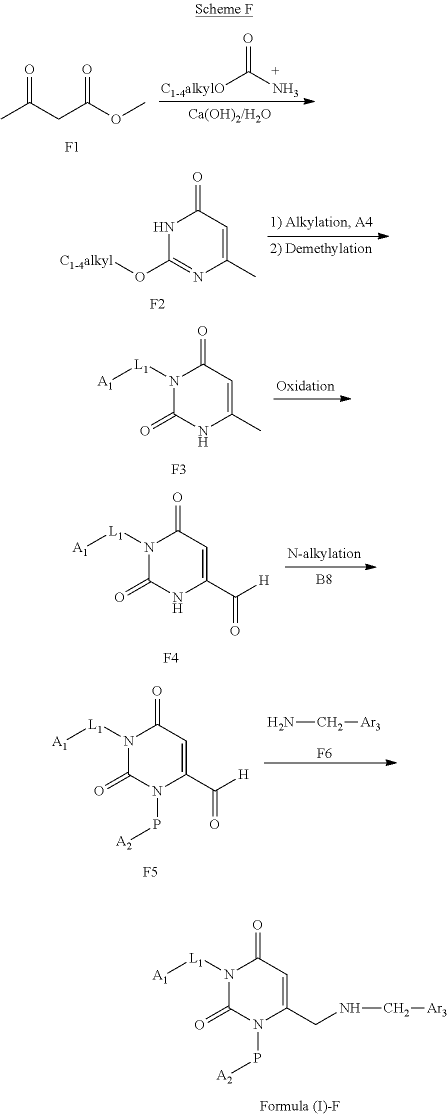

A compound of formula F1 (either commercially available or prepared by known methods described in the scientific literature) may be condensed with an O-alkylated isourea to afford a cyclic compound of formula F2. The amino functionality of a compound of formula F2 may be deprotonated selectively with a base such as lithium hydride and subsequently treated with a compound of formula A4. The O-demethylation of the alkylated compounds formula F2 affords compounds of formula F3. Using conventional oxidation chemistry, the methyl substituent of a compound of formula F3 may be converted to its corresponding aldehyde, affording a compound of formula F4. The secondary amino group may be substituted with —P-A₂ of Formula (I) using coupling chemistry or standard alkylation with a compound of formula B8 to afford a compound of formula F5. A reductive amination with a compound of formula F6 may afford a compound of Formula (I)-F.

Scheme G illustrates the general synthesis of compounds of the present invention wherein Q of Formula (I) is (c) and W is N.

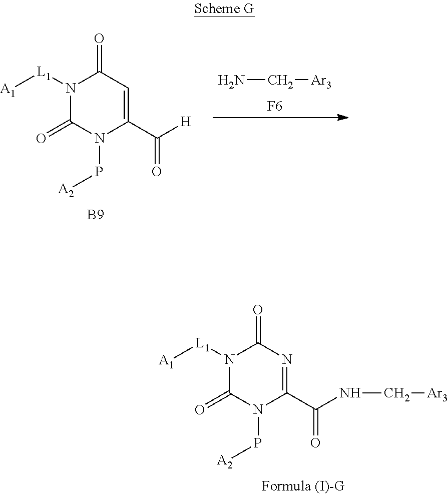

A reductive amination of a compound of formula F6 with a compound of formula B9 may afford a compound of Formula (I)-G.

Scheme H illustrates the general synthesis of compounds of the present invention wherein Q of Formula (I) is (a) or (b) and W is C(R_{W}), wherein R_{W} is C₁₋₂alkyl, and wherein Arₘ is Ar₁ or Ar₂ as previously defined.

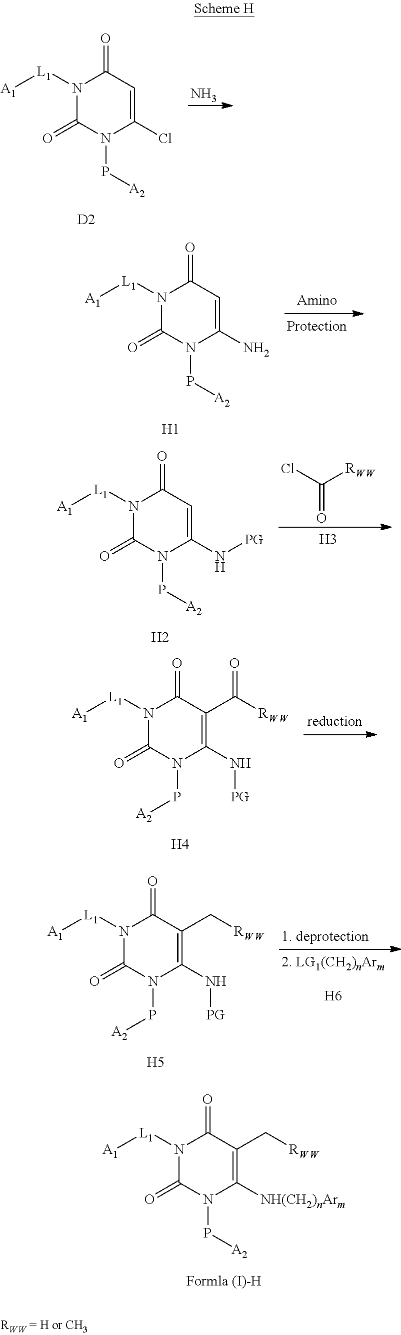

Compound D2 may be reacted with an ammonium salt or an ammonium equivalent to provide a compound of formula H1. The amino functionality of a compound of formula H1 may be protected with an appropriate amino protecting group to provide a compound of formula H2. Acylation of a compound of formula H2 with a compound of formula H3 (wherein R_{WW} may be H or methyl) may give a compound of formula H4. Reduction of the carbonyl group of a compound of formula H4 using standard procedures may provide a compound of formula H5. Removal of the amino protecting group (PG), followed by alkylation of the amino group with a compound of formula H6 provides a compound of Formula (I)-H.

In preparing compounds of Formula (I) wherein A₂ is piperidinyl, a standard protecting group such as N-boc can be used to protect the —NH— in the piperidinyl ring in the synthetic steps shown above. A standard deprotection step can be used after the last step in each scheme to provide compounds of Formula (I) wherein A₂ is piperidinyl.

### SPECIFIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The instant compounds may also be used as intermediates in subsequent examples to produce additional compounds of the present invention. No attempt has been made to optimize the yields obtained in any of the reactions. One skilled in the art would know how to increase such yields through routine variations in reaction times, temperatures, solvents and/or reagents.

Reagents were purchased from commercial sources. Nuclear magnetic resonance (NMR) spectra for hydrogen atoms were measured in the indicated solvent with (TMS) as the internal standard on a Bruker-Biospin Inc. DRX 500 (500 MHz) or DPX 300 (300 MHz) spectrometer. The values are expressed in parts per million downfield from TMS. The mass spectra (MS) were determined on a Micromass Platform LC spectrometer, an Agilent LC spectrometer or a Micromass LCT spectrometer using electrospray techniques. Microwave accelerated reactions were performed using a CEM Discover microwave instrument, and were contained in a sealed pressure vessel unless otherwise noted. Stereoisomeric compounds may be characterized as racemic mixtures or as separate diastereomers and enantiomers thereof using X-ray crystallography and other methods known to one skilled in the art. Unless otherwise noted, the materials used in the examples were obtained from readily available commercial suppliers or synthesized by standard methods known to one skilled in the art of chemical synthesis. The substituent groups, which vary between examples, are hydrogen unless otherwise noted.

### Example 1

### 2-amino-3-methylaminopyridine (Cpd 1a)

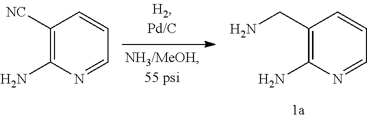

2-Amino-3-methylaminopyridine (Cpd 1a). 2-amino-3-cyanopyridine (3.0 g, 25.2 mmol) was dissolved in 2N NH₃ in methanol (50 mL) and the solution was added to a Parr reaction vessel containing 10% Palladium on charcoal (500 mg) under argon. The reaction was run on a Parr hydrogenation apparatus at 55 psi until the uptake of hydrogen had ceased (˜12 hours). Upon completion, the catalyst was removed via filtration through a pad of diatomaceous earth. The pad was rinsed with methanol (3×50 mL) and the filtrate was reduced in vacuo to provide Compound 1a as a yellow solid. The crude mixture was used in further synthesis without additional purification.

### Example 2

### 3-Aminomethyl-4,6-dimethylpyridine (Cpd 2a)

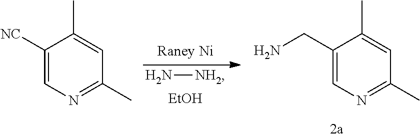

4,6-Dimethylnicotinonitrile (1.0 g, 7.6 mmol) was dissolved in ethanol (35 mL) and the mixture was treated with Raney nickel (5 mL, slurry in water) and hydrazine hydrate (3.8 mL, 75.6 mmol). The solution was stirred overnight at room temperature. Compound 2a was obtained by filtering the reaction mixture through a pad of diatomaceous earth, which was rinsed with methanol (3×50 mL). The filtrate was dried over Na₂SO₄, filtered and concentrated under reduced pressure to afford Compound 2a. The compound was used without additional purification. M+(ES+)=137.1 ¹H NMR (DMSO, d₆) δ 2.35 (s, 3H), 2.42 (s, 3H), 4.01 (s, 2H), 7.13 (s, 1H), 8.42 (s, 1H).

### Example 3

### 3-Aminomethyl-4,6-dimethylpyridine (Cpd 2a)

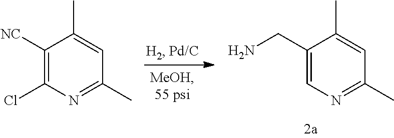

An alternative route for the preparation of compound 2a is described herein. 2-chloro-4,6-dimethylnicotinonitrile (5.0 g, 30 mmol) was dissolved in methanol (50 mL) and the solution was carefully added to a Parr reaction vessel containing 10% Pd on charcoal (500 mg) under argon. The reaction was run on Parr hydrogenation apparatus at 55 psi until uptake of hydrogen had ceased (˜12 h). Upon completion, the catalyst was removed via filtration through a pad of diatomaceous earth. The pad was rinsed with methanol (3×50 mL) and the filtrate was reduced in vacuo to provide Compound 2a. The crude mixture was used in further synthesis without additional purification. MS m/z (ES)=137.1(M+H); ¹H NMR (DMSO, d₆) δ 2.35 (s, 3H), 2.42 (s, 3H), 4.01 (s, 2H), 7.13 (s, 1H), 8.42 (s, 1H).

### Example 4

### 2-amino-3-aminomethyl-4,6-dimethylpyridine (Cpd 4a)

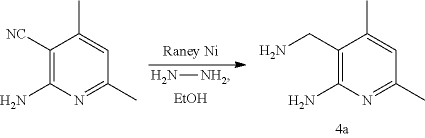

2-Amino-3-aminomethyl-4,6-dimethylpyridine (Cpd 4a). 2-amino-3-cyano-4,6-dimethylpyridine (1.0 g, 6.8 mmol) was dissolved in ethanol (35 mL) and the mixture was treated with Raney nickel (3 mL, slurry in water) and hydrazine hydrate (3.4 mL, 67.9 mmol). The solution was stirred overnight at room temperature. Compound 4a was obtained by filtering the reaction mixture through a pad of diatomaceous earth, which was rinsed with methanol (3×50 mL). The filtrate was dried over Na₂SO₄, filtered and concentrated under reduced pressure to afford Compound 4a. The compound was used without additional purification.

### Example 5

### 6-[(4,6-Dimethyl-pyridin-3-ylmethyl)-amino]-1,3-bis-(4-methoxy-benzyl)-1H-[1,3,5]triazine-2,4-dione (Cpd 22)

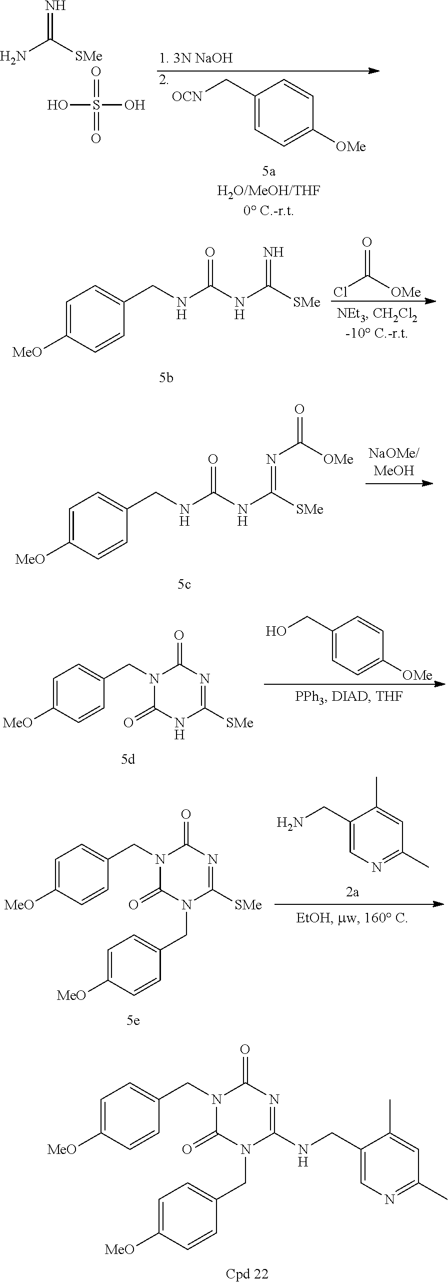

A. ((4-Methoxybenzyl)amino)carbonyl)carbamimidothioic acid methyl ester (Cpd 5b). S-methylisothiouronium sulfate (15.35 g, 55.2 mmol) was dissolved in 8:2:1 MeOH/H₂O/THF (150 mL) and the mixture was treated with 3 N NaOH (18.4 mL, 55.2 mmol). The solution was then cooled to 0° C. and 4-methoxybenzyl isocyanate (Cpd 5a, 9.0 g, 55.2 mmol) was added dropwise over 30 min. The reaction was stirred overnight and gradually warmed to room temperature. The mixture was then washed with saturated aqueous NH₄Cl (100 mL) and extracted with dichloromethane (3×75 mL). The combined organic phases were dried over Na₂SO₄, filtered and concentrated under reduced pressure. The resultant residue was purified by normal phase column chromatograpy (silica gel, 20% EtOAc-100% EtOAc in heptane), to give Compound 5b.

C. 5-(Methylthio)-3,7-dioxo-1-(4-methoxybenzyl)-2-oxa-4,6,8-triazanon-4-en-9-oic acid methyl ester (Cpd 5c). A solution of Compound 5b (7.9 g, 31.2 mmol) in dichloromethane (150 mL) was treated with triethylamine (5.22 mL, 37.4 mmol) and the mixture was cooled to −10° C. Methyl chloroformate (4.79 mL, 62.4 mmol) was added dropwise over 15 min and the reaction was stirred for 4 h while gradually warming to room temperature. The solution was then washed with saturated aqueous NH₄Cl (100 mL) and extracted with dichloromethane (3×75 mL). The combined organic phases were dried over Na₂SO₄, filtered and concentrated. The resultant residue was purified by normal phase column chromatograpy (silica gel, 5% MeOH/95% CH₂Cl₂) to afford Compound 5c.

D. 3-(4-Methoxybenzyl)-6-methylsulfanyl-1H-[1,3,5]-triazine-2,4-dione (Cpd 5d). Compound 5c (8.1 g, 26.0 mmol) was dissolved in MeOH (150 mL) and the solution was treated with NaOMe in MeOH (4.6 M, 10.1 mL, 31.2 mmol) and the reaction was allowed to stir at room temperature for 1 h. A white precipitate formed upon addition of the NaOMe. The reaction mixture was diluted with 1N HCl (50 mL) and the resultant precipitate was collected by vacuum filtration. The solid was dried under reduced pressure at 160° C. over xylenes to afford Compound 5d as its HCl salt.

E. 3-(4-Methoxybenzyl)-1-(4-methoxybenzyl)-6-methylsulfanyl-1H-[1,3,5]-triazine-2,4 dione (Cpd 5e). Compound 5d (4.0 g, 12.7 mmol) was dissolved in THF and was treated with 4-methoxybenzyl alcohol (1.75 g, 12.7 mmol), triphenylphosphine (6.7 g, 25.4 mmol), and diisopropyl azodicarboxylate (2.57 g, 12.7 mmol). The reaction was allowed to stir overnight at room temperature. The solution was partitioned between water (100 mL) and ethyl acetate (3×75 mL). The combined organic layers were dried over anhydrous sodium sulfate, filtered and concentrated under reduced pressure. The crude mixture was purified by normal phase column chromatograpy (silica gel, 20% ethyl acetate-100% ethyl acetate in heptane) to afford Compound 5e.

F. 6-[(4,6-Dimethyl-pyridin-3-ylmethyl)-amino]-1,3-bis-(4-methoxy-benzyl)-1H-[1,3,5]-triazine-2,4-dione (Cpd 22). Compound 5e (100 mg, 0.25 mmol) and Compound 2a (140 mg, 1.0 mmol) were suspended in EtOH (2 mL) and the reaction was irradiated at 160° C. for a total of 60 min in a microwave instrument. The reaction mixture was then reduced under nitrogen and the residue was purified and isolated by reverse phase HPLC to afford Compound 61. MS m/z (ES)=488.3 (M+H); ¹H NMR (DMSO, d₆) δ 2.39 (s, 3H), 2.62 (s, 3H), 3.71 (s, 3H), 3.74 (s, 3H), 4.53 (m, 2H), 4.82 (s, 2H), 5.08 (s, 2H), 6.88 (m, 4H), 7.22 (m, 4H), 7.67 (s, 1H), 8.47 (s, 1H).

Other compounds of the present invention may be prepared by those skilled in the art by varying the starting materials, reagent(s) and conditions used. Using the general procedure of Example 5, the following compounds were prepared:

| Cpd | MS obs | MS calc |
|---|---|---|
| 1 | 513.7 | 513.4 |
| 2 | 499.6 | 499.4 |
| 4 | 478.8 | 479.9 |
| 5 | 478.8 | 479.9 |
| 6 | 475.8 | 476.5 |
| 8 | 463.1 | 463.5 |
| 9 | 525.2 | 525.6 |
| 10 | 476.9 | 477.5 |
| 12 | 544.2 | 544.6 |
| 13 | 543.2 | 543.6 |
| 20 | 545.1 | 545.6 |
| 25 | 554.3 | 554.6 |
| 35 | 511.2 | 511.5 |
| 36 | 503.2 | 503.5 |
| 37 | 502.2 | 502.5 |
| 38 | 529.2 | 529.5 |
| 39 | 460.2 | 460.5 |
| 40 | 460.2 | 460.5 |
| 41 | 460.2 | 460.5 |
| 52 | 488.2 | 488.5 |
| 57 | 551.2 | 551.6 |
| 58 | 505.2 | 505.5 |
| 59 | 474.2 | 474.5 |
| 60 | 476.2 | 476.5 |
| 62 | 474.2 | 474.5 |
| 63 | 473.2 | 473.5 |
| 64 | 528.2 | 528.5 |
| 65 | 474.0 | 474.5 |
| 75 | 491.2 | 491.6 |
| 76 | 446.2 | 446.5 |
| 77 | 485.2 | 485.5 |
| 78 | 455.2 | 455.5 |
| 79 | 439.2 | 439.5 |
| 80 | 475.2 | 475.5 |
| 81 | 470.1 | 470.5 |
| 82 | 490.1 | 490.5 |
| 86 | 473.2 | 473.5 |
| 87 | 529.2 | 529.5 |
| 88 | 470.1 | 470.5 |
| 91 | 517.1 | 517.5 |
| 92 | 475.2 | 475.5 |
| 93 | 503.2 | 503.5 |
| 94 | 489.1 | 489.5 |
| 95 | 476.1 | 476.5 |
| 96 | 524.2 | 524.5 |
| 98 | 529.2 | 529.5 |
| 99 | 542.3 | 542.5 |
| 100 | 504.1 | 504.6 |
| 101 | 459.1 | 459.5 |
| 102 | 498.1 | 498.6 |
| 103 | 452.2 | 452.6 |
| 104 | 489.1 | 489.5 |
| 105 | 542.3 | 542.5 |
| 106 | 488.2 | 488.6 |
| 116 | 476.2 | 476.5 |
| 117 | 492.1 | 493.0 |
| 122 | 527.8 | 528.5 |
| 125 | 487.2 | 487.5 |
| 126 | 485.2 | 485.5 |
| 127 | 484.2 | 484.5 |
| 128 | 500.2 | 500.6 |
| 129 | 498.1 | 498.6 |
| 130 | 497.2 | 497.6 |
| 131 | 523.2 | 523.6 |
| 132 | 536.2 | 536.6 |
| 135 | 517.2 | 517.6 |
| 136 | 533.3 | 533.6 |
| 137 | 520.2 | 520.5 |
| 138 | 484.2 | 484.5 |
| 139 | 497.2 | 497.6 |
| 140 | 501.1 | 501.6 |
| 142 | 514.2 | 514.6 |
| 149 | 481.2 | 481.6 |
| 150 | 494.2 | 494.6 |
| 152 | 603.3 | 603.7 |
| 153 | 468.1 | 468.5 |
| 154 | 474.2 | 474.5 |
| 155 | 512.2 | 512.6 |
| 170 | 484.2 | 484.5 |
| 171 | 484.2 | 484.5 |
| 172 | 497.2 | 497.6 |
| 200 | 505.5 | 505.5 |
| 201 | 474.3 | 474.5 |
| 203 | 493.1 | 493.5 |
| 204 | 506.2 | 506.6 |
| 205 | 493.3 | 493.5 |
| 206 | 506.3 | 506.6 |
| 224 | 483.3 | 483.6 |
| 231 | 479.0 | 478.9 |
| 234 | 473.9 | 473.53 |
| 235 | 527.8 | 527.50 |
| 236 | 527.8 | 527.50 |
| 237 | 528.2 | 527.50 |
| 238 | 443.2 | 466.54 |
| 239 | 469.2 | 468.56 |
| 241 | 519.03 | 518.57 |
| 246 | 590.8 | 590.68 |
| 247 | 475.2 | 474.52 |
| 248 | 489.9 | 489.54 |
| 250 | 608.27 | 608.70 |
| 253 | 487.27 | 487.00 |
| 254 | 453.3 | 452.56 |
| 255 | 521.26 | 521.45 |
| 256 | 459.1 | 458.95 |
| 257 | 491.09 | 490.51 |
| 258 | 508.22 | 507.51 |
| 259 | 532.2 | 531.61 |
| 260 | 533.3 | 532.60 |
| 263 | 516.9 | 516.60 |
| 264 | 528.9 | 528.61 |
| 265 | 559.3 | 558.68 |
| 266 | 464.15 | 463.46 |
| 267 | 473.9 | 473.53 |
| 271 | 453.16 | 452.51 |
| 272 | 465.3 | 464.57 |

### Additional ¹H NMR Data for Compounds of Example 5

6-[(2-Amino-pyridin-3-ylmethyl)-amino]-3-(4-methoxy-benzyl)-1-(5-methoxy-pentyl)-1H-[1,3,5]-triazine-2,4-dione (Cpd 78). ¹H NMR (DMSO, d₆) δ 1.30 (m, 2H), 1.53 (m, 4H), 3.20 (s, 3H), 3.28 (t, 2H, J=6.25 Hz), 3.71 (s, 3H), 3.79 (m, 2H), 4.38 (d, 2H, J=3.88 Hz), 4.80 (s, 2H), 6.86 (m, 3H), 7.23 (d, 2H, J=8.68 Hz), 7.92 (d, 1H, J=5.31 Hz), 8.18 (m, 1H).

6-[(2-Amino-4,6-dimethyl-pyridin-3-ylmethyl)-amino]-1-(1H-indol-4-ylmethyl)-3-(4-methoxy-benzyl)-1H-[1,3,5]triazine-2,4-dione (Cpd 155). ¹H NMR (DMSO, d₆) δ 2.33 (s, 3H), 2.35 (s, 3H), 3.71 (s, 3H), 4.35 (m, 2H), 4.84 (s, 2H), 5.32 (s, 2H), 6.43 (s, 1H), 6.60 (m, 2H), 6.83 (d, 2H, J=8.67 Hz), 7.01 (t, 1H, J=8.15 Hz), 7.24 (d, 2H, J=8.66 Hz), 7.34 (m, 2H), 7.98 (s, 1H), 11.25 (s, 1H).

6-[(2-Amino-4,6-dimethyl-pyridin-3-ylmethyl)-amino]-3-(4-methoxy-benzyl)-1-(5-methoxy-pentyl)-1H-[1,3,5]triazine-2,4-dione (Cpd 224). ¹H NMR (DMSO, d₆) δ 1.25 (m, 2H), 1.47 (m, 4H), 2.37 (s, 3H), 2.49 (s, 3H), 3.19 (s, 3H), 3.25 (t, 2H, J=6.31 Hz), 3.72 (s, 3H), 3.79 (t, 2H, J=6.97 Hz), 4.37 (d, 2H, J=4.30 Hz), 4.80 (s, 2H), 6.69 (s, 1H), 6.86 (d, 2H, J=8.73 Hz), 7.23 (d, 2H, J=8.68 Hz), 7.60 (s, 1H), 7.80 (m, 1H).

### Example 6

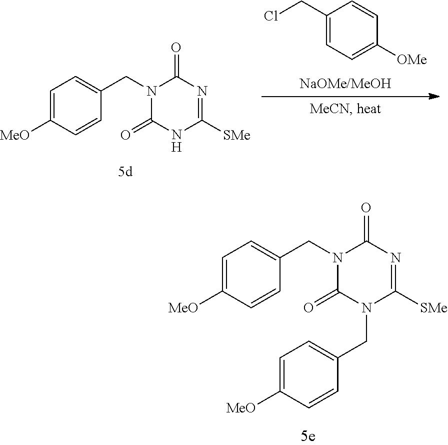

Example 6 describes an alternative route for the preparation of 3-(4-methoxybenzyl)-1-(4-methoxybenzyl)-6-methylsulfanyl-1H-[1,3,5]triazine-2,4 dione, Cpd 5e. Compound 5d (2.0 g, 7.2 mmol) was dissolved in acetonitrile (100 mL) and the reaction mixture was treated with diisopropylethylamine (2.5 mL, 14.3 mmol) and 4-methoxybenzyl chloride (1.35 g, 8.6 mmol). The reaction mixture was then heated to 90° C. and was allowed to stir overnight. Upon cooling, the mixture was partitioned between saturated aqueous NH₄Cl (100 mL) and ethyl acetate (3×75 mL). Combined organic extracts were dried over Na₂SO₄, filtered and reduced. Purification by normal phase column chromatograpy (silica gel, 20% ethyl acetate-100% ethyl acetate in heptane) afforded Compound 5e as a white solid.

### Example 7

### 6-[(2-Amino-4,6-dimethyl-pyridin-3-ylmethyl)-amino]-1,3-bis-(4-methoxy-benzyl)-1H-[1,3,5]triazine-2,4-dione (Cpd 97)

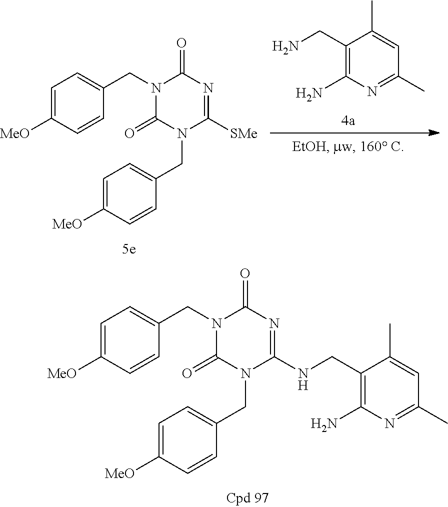

Compound 5e (100 mg, 0.25 mmol) and Compound 4a (76 mg, 0.50 mmol) were suspended in EtOH (2 mL) and the reaction was irradiated at 160° C. for a total of 60 minutes in a microwave instrument. The reaction mixture was then reduced under nitrogen and the resultant residue was purified and isolated by reverse phase HPLC to afford Compound 97. MS m/z (ES)=503.19 (M+H); ¹H NMR (DMSO, d₆) δ 2.35 (s, 3H), 2.36 (s, 3H), 3.72 (s, 3H), 3.73 (s, 3H), 4.36 (d, 2H, J=3.33 Hz), 4.83 (s, 2H), 4.99 (s, 2H), 6.65 (s, 1H), 6.87 (m, 4H), 7.15 (d, 2H, J=8.63 Hz), 7.23 (d, 2H, J=8.61 Hz), 7.62 (s, 2H), 7.97 (m, 1H).

Other compounds of the present invention may be prepared by those skilled in the art by varying the starting materials, reagent(s) and conditions used. Using the general procedure of Example 7, the following compounds were prepared:

| Cpd | MS obs | MS calc |
|---|---|---|
| 157 | 515.2 | 515.6 |
| 212 | 529.3 | 529.6 |
| 213 | 571.4 | 571.7 |

### Example 8

### 3-(2,3-Dihydro-benzofuran-5-ylmethyl)-6-[(4,6-dimethyl-pyridin-3-ylmethyl)-amino]-1-(4-methoxy-benzyl)-1H-[1,3,5]triazine-2,4-dione (Cpd 123)

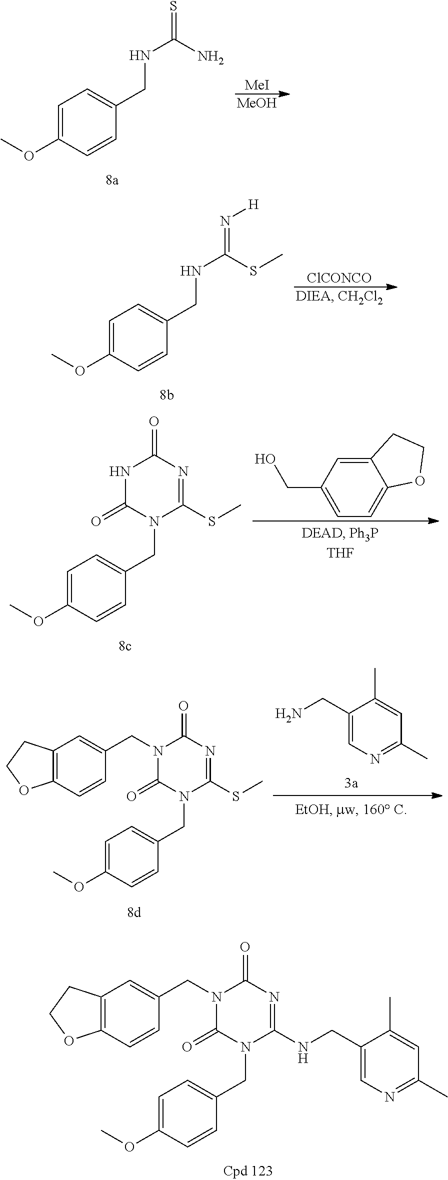

A. 1-(4-Methoxy-benzyl)-6-methylsulfanyl-1H-[1,3,5]triazine-2,4-dione (Cpd 6b). To (4-methoxy-benzyl) thiourea (Cpd 8a, 2.00 g, 10.1 mmol) in MeOH (40 mL) was added methyl iodide (0.64 mL, 10.1 mmol). The reaction was stirred at room temperature for 24 h. The reaction mixture was concentrated to yield crude compound 8b that was used in the next step without further purification.

B. 1-(4-Methoxy-benzyl)-6-methylsulfanyl-1H-[1,3,5]triazine-2,4-dione (Cpd 6c). To Compound 8b (3.6 g, 17.1 mmol) in methylene chloride (40 mL) was added excess diisopropylethylamine (6.61 g, 51.3 mmol). The reaction mixture was cooled to 0° C. A portion of N-chlorocarbonyl isocyanate (1.78 g, 17.1 mmol) was added dropwise. The reaction mixture was allowed to slowly warm to room temperature. After 24 h, water was added and the reaction mixture was extracted with ethyl acetate. The phases were separated, and the organic layer was dried over sodium sulfate, filtered, and concentrated. Methanol was added to the crude product, and the solid was collected by vacuum filtration to give Compound 8c. ¹H NMR (DMSO-d₆) δ 2.45 (3H, s), 3.73 (3H, s), 4.98 (2H, s), 6.89-6.92 (2H, d, J=8.5 Hz), 7.22-7.25 (2H, d, J=8.5 Hz), 11.58 (1H, s).

C. 3-(2,3-Dihydro-benzofuran-5-ylmethyl)-1-(4-methoxy-benzyl)-6-methylsulfanyl-1H-[1,3,5]triazine-2,4-dione (Cpd 8d). To Cpd 8c (0.3 g, 1.07 mmol) in tetrahydrofuran was added 2,3-dihydro-1-benzofuran-5-ylmethanol (0.16 g, 1.07 mmol), triphenylphosphine (0.57 g, 2.15 mmol) and diethyl azodicarboxylate (0.22 g, 1.29 mmol). The reaction was stirred at room temperature for 24 h. The reaction mixture was taken up in ethyl acetate, washed with water, and the phases were separated. The organic layer was dried over sodium sulfate, filtered, and concentrated. The resulting material was purified by normal phase chromatography using an ISCO automated system to give Cpd 8d.

D. 3-(2,3-Dihydro-benzofuran-5-ylmethyl)-6-[(4,6-dimethyl-pyridin-3-ylmethyl)-amino]-1-(4-methoxy-benzyl)-1H-[1,3,5]triazine-2,4-dione (Cpd 8e). Compound 8d (100 mg, 0.24 mmol) and compound 3a (33 mg, 0.25 mmol) were suspended in EtOH (2 mL) and the reaction was irradiated at 160° C. for 60 minutes in a microwave instrument. The reaction mixture was then reduced under nitrogen and the product was purified and isolated by reverse phase HPLC to afford Compound 123. MS m/z (ES)=500.0 (M+H); ¹H NMR (DMSO, d₆) δ 2.49 (3H, s), 2.60 (3H, s), 3.08-3.19 (2H, t, J=8.64 Hz), 3.73 (3H, s), 4.45-4.53 (4H, m), 4.80 (2H,$), 5.05 (2H,$), 6.65-6.68 (1H, d, J=8.18 Hz), 6.87-6.91 (1H, d, J=8.7 Hz), 7.03-7.06 (1H, m), 7.15-7.18 (2H, m), 7.66 (1H, s), 8.30-8.35 (1H, br s), 8.45 (1H, s).

Other compounds of the present invention may be prepared by those skilled in the art by varying the starting materials, reagent(s) and conditions used. Using the general procedure of Example 8, the following compounds were prepared:

| Cpd | MS obs | MS calc |
|---|---|---|
| 45 | 529.1 | 529.5 |
| 46 | 489.3 | 489.5 |
| 47 | 490.2 | 490.5 |
| 48 | 515.2 | 515.6 |
| 49 | 513.2 | 513.5 |
| 55 | 463.2 | 463.5 |
| 56 | 503.3 | 503.5 |
| 107 | 501.9 | 502.6 |
| 108 | 503.0 | 503.5 |
| 109 | 527.8 | 528.6 |
| 110 | 525.9 | 526.5 |
| 111 | 488.0 | 488.6 |
| 112 | 475.9 | 476.5 |
| 113 | 458.9 | 459.5 |
| 114 | 515.8 | 516.6 |
| 124 | 519.9 | 520.5 |
| 133 | 497.9 | 498.6 |
| 134 | 484.9 | 485.5 |
| 143 | 474.9 | 475.5 |
| 144 | 487.9 | 488.6 |
| 145 | 500.9 | 501.6 |
| 146 | 513.9 | 514.6 |

### Example 9

### 6-[(2-Amino-pyridin-3-ylmethyl)-amino]-3-(4-hydroxy-benzyl)-1-(4-methoxy-benzyl)-1H-[1,3,5]triazine-2,4-dione (Cpd 54)

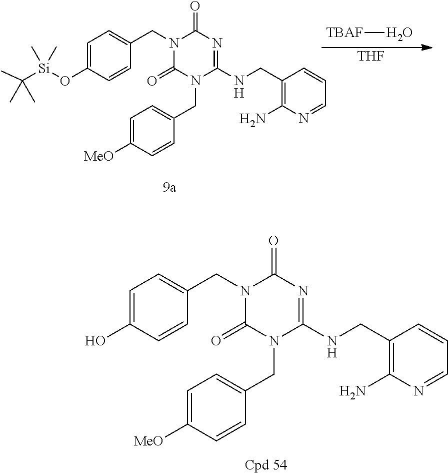

A. 6-[(2-Amino-pyridin-3-ylmethyl)-amino]-3-[4-(tert-butyl-dimethyl-silanyloxy)-benzyl]-1-(4-methoxy-benzyl)-1H-[1,3,5]-triazine-2,4-dione (Cpd 9a) (150 mg, 0.26 mmol) was prepared according to the methods described in Example 8, and substituting [4-(tert-butyl-dimethyl-silanyloxy)-phenyl]-methanol for 2,3-dihydro-1-benzofuran-5-ylmethanol in Step C.

B. 6-[(2-Amino-pyridin-3-ylmethyl)-amino]-3-(4-hydroxy-benzyl)-1-(4-methoxy-benzyl)-1H-[1,3,5]triazine-2,4-dione (Cpd 54). Compound 7a was suspended in THF (3 mL) and the reaction mixture was treated with tetrabutylammonium fluoride monohydrate (82 mg, 0.31 mmol). The solution was stirred at room temperature overnight. The mixture was then concentrated under nitrogen and the residue was purified by reverse phase HPLC to give the title compound 54. MS m/z (ES)=461.1 (M+H).

Other compounds of the present invention may be prepared by those skilled in the art by varying the starting materials, reagent(s) and conditions used. Using the general procedure of Example 9, the following compounds were prepared:

| Cpd | MS obs | MS calc |
|---|---|---|
| 181 | 510.2 | 510.5 |

### Example 10

### 6-[(6-Amino-pyridin-2-ylmethyl)-amino]-1,3-bis-(4-methoxy-benzyl)-1H-[1,3,5]triazine-2,4-dione (Cpd 115)

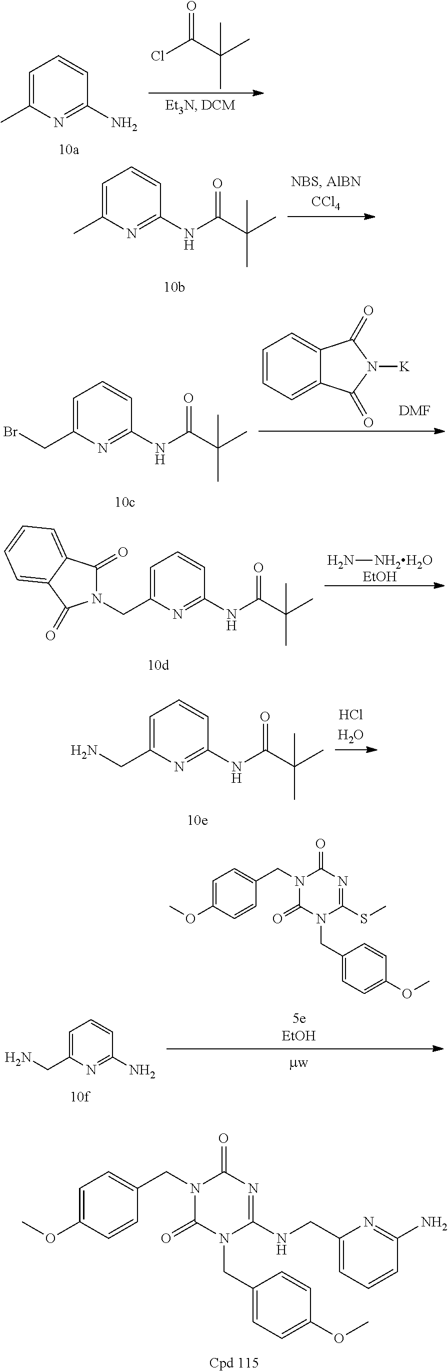

A. 2,2-Dimethyl-N-(6-methyl-pyridin-2-yl)-propionamide (Cpd 10b) To a mixture of 2-amino-6-methylpyridine 10a (500 mg, 4.6 mmol), and triethylamine (778 μL, 5.98 mmol) in dichloromethane (50 mL) was added pivaloyal chloride (628 μL, 5.1 mmol). The mixture was allowed to stir at room temperature for three hours. The mixture was washed with saturated sodium bicarbonate followed by brine. The organic extract was dried over magnesium sulfate and concentrated to give Compound 10b (876 mg) as a crude oil, which solidified upon standing.

B. N-(6-Bromomethyl-pyridin-2-yl)-2,2-dimethyl-propionamide (Cpd 10c) A mixture of compound 10b, (776 mg, 4.03 mmole), N-bromosuccinimide (NBS) (431 mg, 2.4 mmol), and 2,2′-azobisisobutyronitrile (66 mg, 0.4 mmol) in carbon tetrachloride (100 mL) was heated to 90° C. for 2.5 hours. LC analysis indicated a mixture of the desired product, undesired di-bromonated material and starting material. The mixture was cooled to room temperature, washed with saturated sodium bicarbonate and brine. The organic extract was dried over magnesium sulfate and concentrated to yellow oil. The oil was purified by normal phase chromatography, eluting with 10-30% ethyl acetate in heptane to yield compound 10c. MS m/z (ES)=193.2 (M+H).

C. N-[6-(1,3-Dioxo-1,3-dihydro-isoindol-2-ylmethyl)-pyridin-2-yl]-2,2-dimethyl-propionamide (Cpd 10d) A mixture of compound 10c (335 mg, 1.24 mmol) and potassium phthalamide (230 mg, 1.24 mmol) in DMF (3 mL) was heated to 160° C. in an oil bath for 4 hours. The mixture was cooled to room temperature and allowed to stir overnight. The mixture was diluted with water (100 mL) and extracted 2× with ethyl acetate. The combined organic extracts were washed with water, dried over magnesium sulfate and concentrated to a yellow oil-solid. This material was purified by normal phase chromatography, eluting with 30-50% ethyl acetate in heptane to give compound 10d. MS m/z (ES)=338.1 (M+H).

D. N-(6-Aminomethyl-pyridin-2-yl)-2,2-dimethyl-propionamide (Cpd 10e). A mixture of compound 10d (200 mg, 0.59 mmol), and hydrazine monohydrate (29 μL, 0.59 mmol) in ethanol (10 mL) was heated to 90° C. for six hours then cooled to rt and allowed to stir overnight. LC analysis indicated the reaction was incomplete so an additional 5 μL of hydrazine monohydrate was added and the mixture was heated to 90° C. for 22 h. The mixture was concentrated, and the resultant residue was taken up in ethyl acetate, giving a white precipitate. The precipitate was removed by filtration, and the filtrate was concentrated and then purified by reverse phase liquid chromatography to afford Compound 10e. MS m/z (ES)=208.1 (M+H). ¹H NMR (MeOD, d₄). δ 1.25 (s, 9H), 4.12 (s, 3H), 7.18 (d, 1H, J=7.7 Hz), 7.84 (t, 1H, J=8.0, 7.8 Hz), 8.01-8.04 (d, 1H, J=8.0 Hz).

E. 6-Aminomethyl-pyridin-2-ylamine (Cpd 10f). To a solution of compound 10e (100 mg, 0.48 mmol) in water (10 mL) was added concentrated HCl (500 μL, 12M). The mixture was heated to reflux for 30 minutes. After cooling to rt, the solution was allowed to stir overnight. Nitrogen gas was bubbled through the solution for one hour. The solution was then lyophilized fto obtain compound 10f. MS m/z (ES)=124.1 (M+H).

F. 6-[(6-Amino-pyridin-2-ylmethyl)-amino]-1,3-bis-(4-methoxy-benzyl)-1H-[1,3,5]triazine-2,4-dione (Cpd 115). A mixture of compound 5e (168 mg, 0.42 mmol), compound 10f (95 mg, 0.42 mmol), diisopropylethylamine (187 μL, 1.7 mmol) and ethanol (3 mL) was irradiated at 140° C. for 20 minutes in a microwave instrument. Subsequently, the mixture was irradiated at 160° C. for 20 minutes in a microwave instrument. The resulting mixture was purified by reverse phase HPLC to give compound 115 as its TFA salt. MS m/z (ES)=474.9 (M+H). ¹H NMR (DMSO, d₆). δ 3.65 (s, 3H), 3.74 (s, 3H), 4.44 (s, 2H), 4.64 (s, 2H), 5.01 (s, 2H), 6.32 (d, 1H, J=7.3 Hz), 6.71 (d, 1H, J=8.7 Hz), 6.79 (d, 2H, J=8.7 Hz), 6.86 (d, 2H, J=8.7 Hz), 7.14-7.18 (dd, 4H, J=5.2, 5.2 Hz), 7.72 (t, 1H, J=7.6, 8.4 Hz), 7.71-7.75 (bs, 2H), 8.33 (s, 1H).

### Example 11

### 1,3-Bis-(4-methoxy-benzyl)-6-[(6-propylamino-pyridin-2-ylmethyl)-amino]-1H-[1,3,5]-triazine-2,4-dione, (Cpd 147)

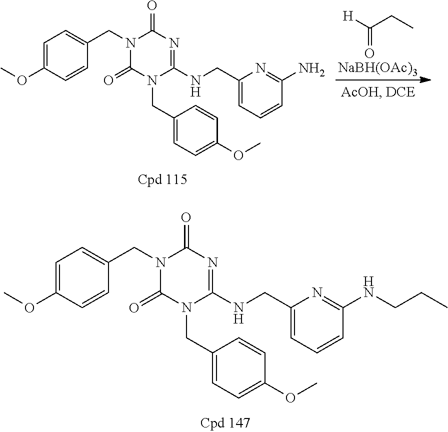

A. 1,3-Bis-(4-methoxy-benzyl)-6-[(6-propylamino-pyridin-2-ylmethyl)-amino]-1H-[1,3,5]triazine-2,4-dione (Cpd 147). A mixture of Compound 115 (30 mg, 0.13 mmol), propionaldehyde (5.8 μL, 0.086 mmol), sodium triacetoxyborohydride (18 mg, 0.086 mmol) and acetic acid (12 μL, 0.215 mmol) in dichloroethane (5 mL) was allowed to stir at room temperature. After four days, an additional 10 μL of propionaldehyde was added. After stirring an additional day, another 10 μL of propionaldehyde as added. The reaction was washed with saturated sodium bicarbonate and brine. The organic layer was dried over magnesium sulfate, filtered, and the filtrate was concentrated. The concentrate was purified by reverse phase chromatography to obtain compound 147 as its TFA salt. MS m/z (ES)=516.9 (M+H).

### Example 12

### 6-[(6-Amino-pyridin-2-ylmethyl)-amino]-1,3-bis-(4-methoxy-benzyl)-1H-pyrimidine-2,4-dione (Cpd 148)

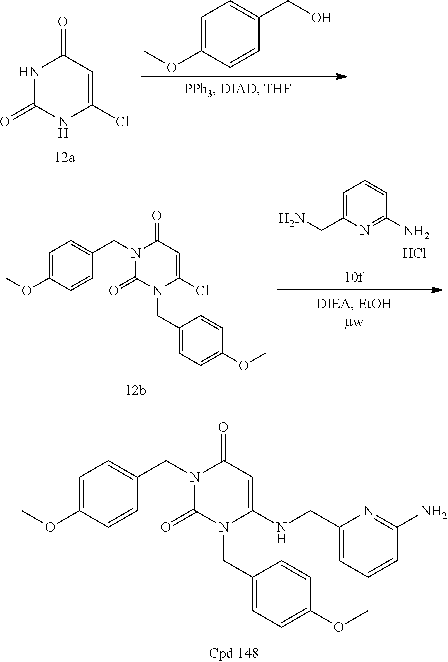

A. 6-Chloro-1,3-bis-(4-methoxy-benzyl)-1H-pyrimidine-2,4-dione (Cpd 10b). A solution of 6-chlorouracil 12a, (500 mg, 3.4 mmol), 4-methoxybenzyl alcohol (990 mg, 7.2 mmol), triphenylphosphine (2.9 g, 11.2 mmol), diisopropylazodicarboxylate (1.6 mL, 8.2 mmol) in THF (100 mL) was allowed to stir at room temperature overnight. The solution was concentrated. The concentrate was taken up in ethyl acetate and washed with saturated sodium bicarbonate and brine. The organic layer was dried over magnesium sulfate, filtered, and the filtrate was concentrated. The concentrate was purified by reverse phase chromatography to afford compound 12b. MS m/z (ES)=386.9 (M+H). ¹H NMR (MeOD, d₄). δ 3.75 (s, 3H), 3.76 (s, 3H), 5.01 (s, 2H), 5.21 (s, 2H), 5.99 (s, 1H), 6.82-6.88 (dd, 4H, J=8.9, 8.9 Hz), 7.22 (d, 2H, 8.5 Hz), 7.32 (d, 2H, J=8.9 Hz).

B. 6-[(6-Amino-pyridin-2-ylmethyl)-amino]-1,3-bis-(4-methoxy-benzyl)-1H-pyrimidine-2,4-dione (Cpd 12c). A suspension of compound 10f, (50 mg, 0.13 mmol), compound 12b (25 mg, 0.13 mmol), diisopropylethylamine (57 μL, 0.52 mmol) in ethanol (3 mL) was irradiated at 140° C. for 20 minutes in a microwave instrument. The mixture was concentrated and the residue purified by reverse phase chromatography to obtain compound 148 as its TFA salt. MS m/z (ES)=473.9 (M+H). ¹H NMR (DMSO, d₆). δ 3.72 (s, 6H), 4.23 (bs, 2H), 4.77 (s, 2H), 5.12 (s, 2H), 6.78 (d, 1H, J=9.4 Hz), 6.88 (m, 1H), 6.81 (d, 2H, J=8.4 Hz), 6.91 (d, 2H, J=9.0 Hz), 7.22 (dd, 4H, J=8.9, 8.9 Hz), 7.40 (t, 1H, J=5.4, 5.4 Hz), 7.72 (t, 1H, J=8.4, 7.9 Hz).

Other compounds of the present invention may be prepared by those skilled in the art by varying the starting materials, reagent(s) and conditions used. Using the general procedure of Example 12, the following compounds were prepared:

| Cpd | MS obs | MS calc |
|---|---|---|
| 26 | 474.3 | 474.5 |
| 61 | 487.2 | 487.6 |

### Example 13

### 3-(4-Fluoro-benzyl)-1-(4-methoxy-benzyl)-6-[(5,6,7,8-tetrahydro-1,8]naphthyridin-2-ylmethyl)-amino]-1H-[1,3,5]triazine-2,4-dione (Cpd 21)

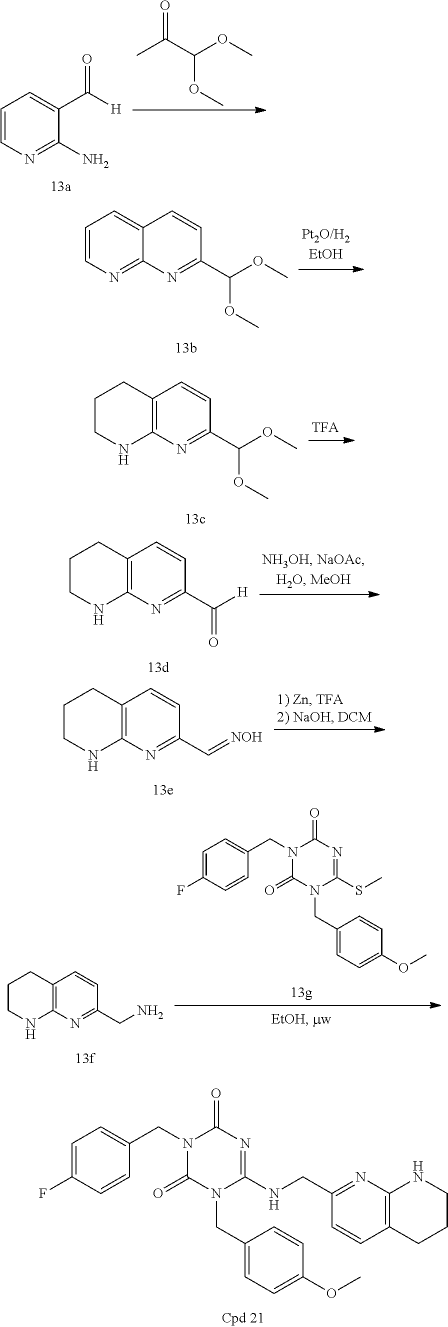

A. 2-Dimethoxymethyl-[1,8]naphthyridine (Cpd 13b). A solution of 2-amino-3-pyridine carboxaldehyde (13a, 50 mg, 4.1 mmol), pyruvic aldehyde dimethyl acetal (641 μL, 5.3 mmol), 3N sodium hydroxide (1.8 mL, 5.3 mmol), ethanol (50 mL) and water (5 mL) was allowed to stir at room temperature overnight. The mixture was concentrated and the residue partitioned between ethyl acetate and brine. The organic layer was dried over magnesium sulfate, filtered, and the filtrate was concentrated to obtain 13b.

B. 7-Dimethoxymethyl-1,2,3,4-tetrahydro-[1,8]naphthyridine (Cpd 13c). A mixture of 13b (0.8 g, 3.9 mmol) and platinum oxide (27 mg, 0.12 mmol) in ethanol (100 mL) was placed under a hydrogen atmosphere at atmospheric pressure for 22 hours. The mixture was filtered through a pad of diatomaceous earth and the filtrate was concentrated to obtain product 13c (0.73 g) as a white solid.

C. 5,6,7,8-Tetrahydro-[1,8]naphthyridine-2-carbaldehyde (Cpd 13d). Compound 13c (0.73 g) was dissolved in trifluoroacetic acid (5 mL). The resulting mixture was allowed to stir at room temperature under argon for 1.5 hours. The mixture was concentrated. The residue was dissolved in methylene chloride and washed 2× with saturated sodium bicarbonate solution. The organic layer was dried over magnesium sulfate, filtered, and the filtrate was concentrated to obtain compound 13d.

D. 5,6,7,8-Tetrahydro-[1,8]naphthyridine-2-carbaldehyde oxime (Cpd 13e). A solution of hydroxylamine hydrochloride (0.46 g, 6.6 mmol), and sodium acetate trihydrate (0.90 g, 6.6 mmol) in water (50 mL) was heated to 60° C. To this mixture was added dropwise, a solution of 13d (0.54 g, 3.3 mmol) in methanol (50 mL). After stirring for 2 hours, the mixture was concentrated to approximately 50 mL. The residue was diluted with saturated sodium sulfate and extracted 2× with ethyl ether. The combined organic extracts were washed with saturated sodium bicarbonate solution, dried over sodium sulfate and concentrated to obtain compound 13e.

E. C-(5,6,7,8-Tetrahydro-[1,8]naphthyridin-2-yl)-methylamine (Cpd 13f). To a solution of 13e (0.46 g, 2.6 mmol) in trifluoroacetic acid (10 mL) was added zinc dust (0.95 g, 15 mmol). The mixture was stirred vigorously for 20 minutes. The resulting solution was poured into a mixture of 3N sodium hydroxide (43 mL, 130 mmol), and methylene chloride (50 mL) that was cooled in an ice bath. After warming to room temperature, the mixture was filtered through a pad of diatomaceous earth and rinsed with additional dichloromethane and water. The phases of the filtrate were separated. The organic layer was dried over sodium sulfate, filtered, and concentrated obtain the compound 13f. MS m/z (ES)=164.1 (M+H). ¹H NMR (CDCl₃). δ 1.56-1.82 (bs, 2H), 1.91 (q, 2H, J=6.6, 5.9, 5.5, 6.6 Hz), 2.70 (t, 2H, J=6.2, 6.2 Hz), 3.40 (m, 2H), 3.71 (s, 2H), 4.84 (bs, 1H), 6.44 (d, 1H, J=7.2 Hz), 7.10 (d, 1H, J=7.2 Hz).

F. 3-(4-Fluoro-benzyl)-1-(4-methoxy-benzyl)-6-methylsulfanyl-1H-[1,3,5]triazine-2,4-dione (Cpd 13g). Compound 13g was obtained using the procedure described in Example 8, Step C, substituting 4-fluorobenzyl alcohol for 2,3-dihydro-1-benzofuran-5-ylmethanol.

G. 3-(4-Fluoro-benzyl)-1-(4-methoxy-benzyl)-6-[(5,6,7,8-tetrahydro-[1,8]naphthyridin-2-ylmethyl)-amino]-1H-[1,3,5]triazine-2,4-dione (Cpd 21). A mixture of 13g (50 mg, 0.13 mmol) and compound 13f (42 mg, 0.26 mmol) in ethanol (2 mL) was irradiated at 140° C. in a microwave instrument for two 20 minute cycles. The resulting mixture was concentrated and purified by reverse phase chromatography to obtain the desired compound 21. MS m/z (ES)=503.3 (M+H). ¹H NMR (DMSO-d₆). δ 1.81 (bs, 2H), 2.72 (bs, 2H), 3.40 (bs, 2H), 4.49 (bs, 2H), 4.88 (s, 2H), 5.08 (s, 2H), 6.31-6.34 (d, 2H, J=7.3 Hz), 6.94 (d, 2H, J=8.7 Hz), 7.10-7.23 (m, 4H), 7.31-7.36 (m, 2H), 7.52 (d, 1H, J=7.3 Hz), 7.99 (bs, 1H), 8.40 (bs, 1H).

### Example 14

### 1,3-Bis-(4-methoxy-benzyl)-6-(pyridin-3-ylmethoxy)-1H-pyrimidine-2,4-dione (Cpd 121)

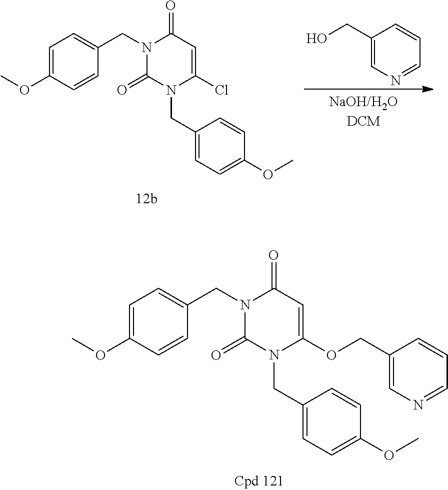

A solution of 12b (50 mg, 0.13 mmol) in dichloromethane (3 mL) was added to a mixture of pyridine 3-methanol (25 μL, 0.26 mmol), benzyltriethylammonium chloride (3 mg, 0.13 mmol) in 1N sodium hydroxide solution (2.6 mL). After stirring at room temperature for 24 hours, an additional 100 μL, of pyridine 3-methanol was added. After stirring an additional 24 hours, the reaction mixture was separated, the organic layer dried over magnesium sulfate, filtered, and the filtrate was concentrated. The concentrate was purified by reverse phase chromatography to obtain Compound 121. MS m/z (ES)=459.9 (M+H). ¹H NMR (DMSO-d₆). δ 3.71 (s, 6H), 4.92 (d, 4H, J=7.8 Hz), 5.29 (s, 2H), 5.45 (s, 1H), 6.84 (t, 4H, J=8.73, 8.91), 7.09 (d, 2H, J=8.74 Hz), 7.23 (d, 2H, J=8.61 Hz), 7.55 (q, 1H, J=5.04, 2.77, 5.07 Hz), 7.86 (d, 1H, J=7.99 Hz), 8.63 (s, 2H).

Other compounds of the present invention may be prepared by those skilled in the art by varying the starting materials, reagent(s) and conditions used. Using the general procedure of Example 14, the following compounds were prepared:

| Cpd | MS obs | MS calc |
|---|---|---|
| 190 | 474.9 | 475.5 |
| 202 | 503.3 | 503.6 |
| 225 | 488.9 | 489.5 |
| 232 | 476.2 | 475.5 |

### Example 15

### (3-Aminomethyl-pyridin-2-yl)-(2-methoxy-ethyl)-amine (Cpd 15c)

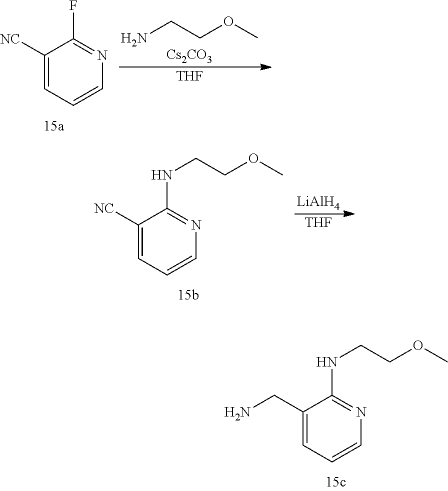

A. 2-(2-Methoxy-ethylamino)-nicotinonitrile (Cpd 15b) To a solution of 3-cyano-2-fluoropyridine (15a) (100 mg, 0.82 mmol) in tetrahydrofuran (1.6 mL) was added cesium carbonate (267 mg, 0.82 mmol) and 2-methoxyethylamine (68 mg, 0.9 mmol). The mixture was stirred at room temperature for 18 h, and then concentrated. The residue was taken up in dichloromethane/water, absorbed onto diatomaceous earth, and eluted with dichloromethane. The eluate was concentrated to provide compound 15b.

B. (3-Aminomethyl-pyridin-2-yl)-(2-methoxy-ethyl)-amine (Cpd 15c) To a cooled (0° C.) solution of lithium aluminum hydride (0.82 mL, 1M solution in tetrahydrofuran, 0.82 mmol) was added compound 15b in tetrahydrofuran (1 mL). The reaction mixture was stirred at 0° C. for 15 min, then stirred at room temperature for 1 h. After successively quenching with water (0.15 mL), sodium hydroxide (0.15 mL, 2N solution in water), and water (0.15 mL) the mixture was filtered and concentrated to furnish compound 15c.

### Example 16

### 3-(4-Fluoro-benzyl)-1-(4-methoxy-benzyl)-6-{[2-(2-methoxy-ethylamino)-pyridin-3-ylmethyl]-amino}-1H-[1,3,5]triazine-2,4-dione (Cpd 28)

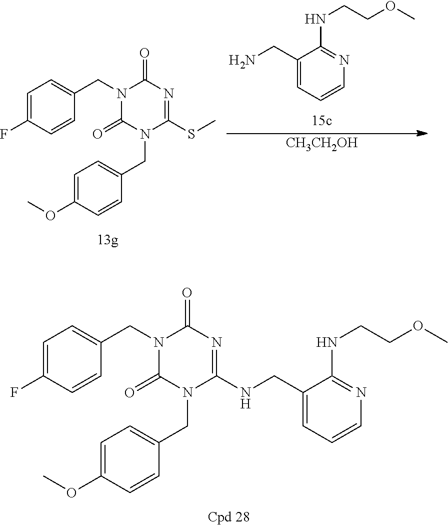

To a reaction vessel containing compound 13g (40 mg, 0.1 mmol) in ethanol (0.75 mL) was added compound 15c (36 mg, 0.2 mmol). The mixture was irradiated at 180° C. in a microwave instrument for two 30 min intervals, then concentrated. The residue was dissolved in methyl sulfoxide and purified by reverse phase chromatography to furnish the title compound 28 as its trifluoroacetate salt. ¹H NMR (methanol-d₄): δ 7.78 (d, 1H, J=4.9 Hz), 7.68 (d, 1H, J=5.8 Hz), 7.46 (m, 2H), 7.12 (d, 2H, J=8.7 Hz), 7.02 (t, 2H, J=8.8 Hz), 6.85-6.80 (m, 3H), 5.10 (s, 2H), 5.03 (s, 2H), 4.57 (s, 2H), 3.75 (s, 3H), 3.59 (m, 4H), 3.19 (s, 3H); HRMS m/z (M+H)⁺ calcd for C₂₇H₃₀FN₆O₄ 521.2313, found 521.2302.

Other compounds of the present invention may be prepared by those skilled in the art by varying the starting materials, reagent(s) and conditions used. Using the general procedure of Example 16, the following compounds were prepared:

| Cpd | MS obs | MS calc |
|---|---|---|
| 11 | 517.1 | 517.6 |
| 15 | 505.2 | 505.6 |
| 17 | 533.2 | 533.6 |
| 18 | 549.2 | 549.6 |
| 19 | 491.2 | 491.5 |
| 27 | 534.2 | 534.6 |
| 29 | 507.2 | 507.5 |
| 30 | 506.1 | 506.6 |
| 31 | 545.1 | 545.6 |
| 34 | 517.3 | 517.6 |
| 50 | 533.2 | 533.6 |
| 51 | 546.2 | 546.6 |
| 66 | 549.2 | 549.7 |
| 67 | 545.3 | 545.7 |
| 68 | 559.1 | 559.6 |
| 69 | 555.1 | 555.6 |
| 70 | 586.2 | 586.7 |
| 71 | 517.2 | 517.6 |
| 72 | 533.0 | 533.6 |
| 73 | 561.2 | 561.6 |
| 74 | 562.2 | 562.6 |

### Example 17

### 6-[(2-Amino-pyridin-3-ylmethyl)-amino]-3-[2-(4-fluoro-phenoxy)-ethyl]-1-(4-methoxy-benzyl)-1H-[1,3,5]triazine-2,4-dione (Cpd 141)

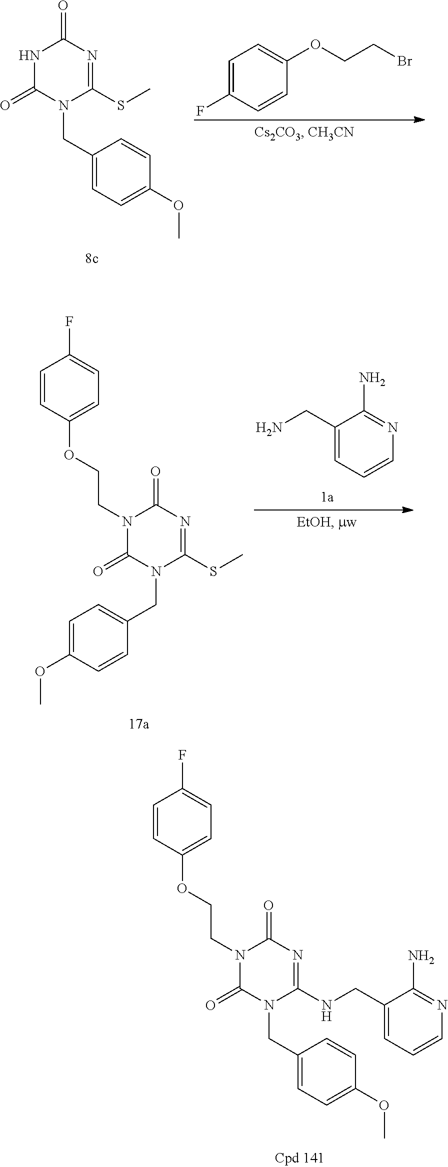

A. 3-[2-(4-Fluoro-phenoxy)-ethyl]-1-(4-methoxy-benzyl)-6-methylsulfanyl-1H-[1,3,5]triazine-2,4-dione (Cpd 17a). To a reaction vessel containing compound 8c (28 mg, 0.1 mmol) in acetonitrile (0.5 mL) was added cesium carbonate (32 mg, 0.1 mmol) and 1-(2-bromo-ethoxy)-4-fluoro-benzene (17.1 mg, 0.1 mmol). The mixture was stirred at room temperature for 16 h, then concentrated. The residue was taken up in dichloromethane/water, absorbed onto diatomaceous earth, and eluted with dichloromethane. The eluate was concentrated to provide compound 17a.

B. 6-[(2-Amino-pyridin-3-ylmethyl)-amino]-3-[2-(4-fluoro-phenoxy)-ethyl]-1-(4-methoxy-benzyl)-1H-[1,3,5]triazine-2,4-dione (Cpd 141). To Compound 17a in ethanol (0.5 mL) was added Compound 1a (18 mg, 0.15 mmol). The mixture was irradiated at 180° C. in a microwave instrument for two 30 min intervals, then concentrated. The residue was dissolved in methyl sulfoxide and purified by reverse phase chromatography to furnish the title compound 141 as its trifluoroacetate salt. ¹H NMR (methanol-d₄): δ 7.80 (d, 1H, J=4.8 Hz), 7.61 (d, 1H, J=5.8 Hz), 7.17 (s, 1H), 7.14 (s, 1H), 6.98-6.79 (m, 8H), 5.12 (s, 2H), 4.50 (s, 2H), 4.28 (m, 2H), 4.22 (m, 2H), 3.77 (s, 3H); HRMS m/z (M+H)⁺ calcd for C₂₅H₂₆FN₆O₄ 493.2000, found 493.1999.

Other compounds of the present invention may be prepared by those skilled in the art by varying the starting materials, reagent(s) and conditions used. Using the general procedure of Example 17, the following compounds were prepared:

| Cpd | MS obs | MS calc |
|---|---|---|
| 23 | 485.1 | 485.5 |
| 24 | 491.1 | 491.6 |
| 42 | 475.2 | 475.5 |
| 43 | 445.2 | 445.5 |
| 44 | 470.1 | 470.5 |
| 60 | 476.2 | 476.5 |
| 83 | 524.0 | 524.5 |
| 84 | 510.9 | 511.5 |
| 89 | 571.1 | 571.4 |
| 90 | 511.1 | 511.6 |
| 119 | 498.2 | 498.6 |
| 120 | 503.0 | 503.5 |
| 156 | 499.2 | 499.5 |
| 197 | 468.2 | 468.6 |
| 207 | 502.2 | 502.5 |
| 209 | 516.3 | 516.6 |
| 216 | 513.2 | 513.6 |
| 217 | 516.1 | 516.6 |
| 218 | 506.2 | 506.6 |
| 220 | 517.1 | 517.6 |
| 222 | 528.2 | 528.6 |
| 229 | 497.2 | 497.6 |
| 230 | 484.2 | 484.5 |

### Additional ¹H NMR Data for Compounds of Example 17

6-[(2-Amino-4,6-dimethyl-pyridin-3-ylmethyl)-amino]-1-(4-methoxy-benzyl)-3-(1-methyl-1H-benzotriazol-5-ylmethyl)-1H-[1,3,5]-triazine-2,4-dione (Cpd 222). ¹H NMR (methanol-d₄): 7.97 (s, 1H), 7.70 (m, 2H), 7.32 (d, 1H, J=8.7 Hz), 7.08 (d, 1H, J=8.7 Hz), 6.84 (m, 2H), 6.61 (s, 1H), 5.23 (s, 2H), 5.14 (s, 2H), 4.51 (s, 2H), 4.32 (s, 3H), 3.75 (s, 3H), 2.40 (s, 3H), 2.26 (s, 3H); HRMS m/z (M+H)⁺ calcd for C₂₂H₃₀N₉O₃ 528.2472, found 517.2468.

### Example 18

### 1-(4-Difluoromethoxy-benzyl)-6-[(4,6-dimethyl-pyridin-3-ylmethyl)-amino]-3-(4-fluoro-benzyl)-1H-[1,3,5]triazine-2,4-dione (Cpd 160)

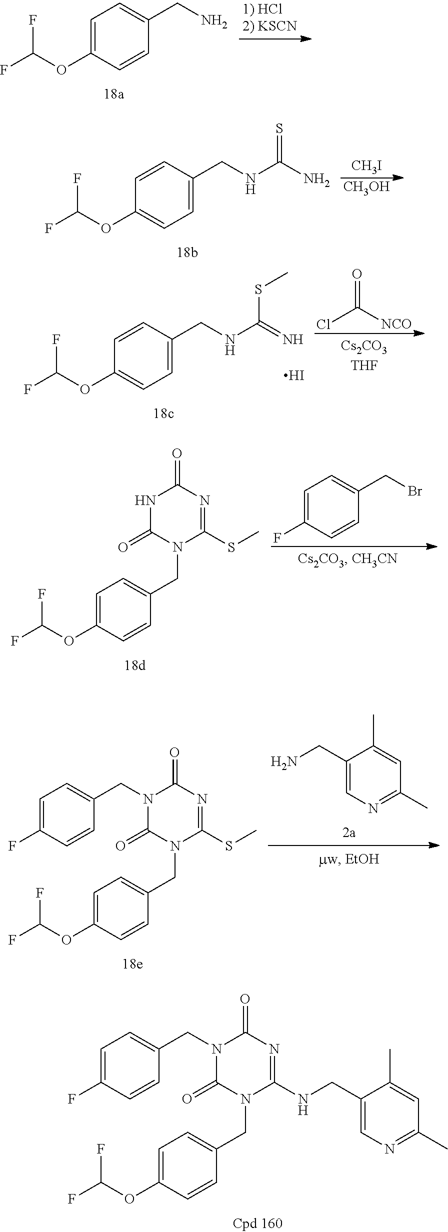

A. (4-Difluoromethoxy-benzyl)-thiourea (18b). To a solution of compound 18a (2.0 g, 11.6 mmol) in dichloromethane (12 mL) at −78° C. was added ethereal hydrogen chloride (24 mL, 1.0 M solution in ethyl ether, 24 mmol). The mixture was allowed to warm to room temperature, then concentrated. To the resulting residue in 1,4-dioxane (32 mL) was added potassium isothiocyanate (1.7 g, 17.3 mmol). The mixture was stirred at reflux for 16 h, then concentrated. The residue was taken up in tetrahydrofuran (25 mL), poured into water (50 mL), and the layers separated. The aqueous layer was extracted with ethyl acetate (3×) and the combined organic layer was washed with 1N HCl and brine. The organic layer was dried over magnesium sulfate, filtered, and the filtrate was concentrated to provide compound 18b.

B. (4-Difluoromethoxy-benzyl)-thiourea hydroiodide (Cpd 18c). A mixture of Compound 18b (2.44 g, 10.5 mmol), iodomethane (1.8 g, 12.6 mmol), and methanol (13 mL) was stirred at room temperature for 18 h, then concentrated to a residue to provide Compound 18c, which was used without further purification in subsequent reactions.

C. 1-(4-Difluoromethoxy-benzyl)-6-methylsulfanyl-1H-[1,3,5]triazine-2,4-dione (Cpd 18d). To compound 18c in tetrahydrofuran (35 mL) was added cesium carbonate (17.1 g, 52.5 mmol). After cooling the mixture to 0° C., N-chloro-carbonyl isocyanate (4.4 g, 42 mmol) was added and the reaction mixture was stirred vigorously for 18 h, then concentrated. The resulting residue was taken up in dichloromethane and water and the layer was separated. The aqueous layer was extracted with dichloromethane and the combined organic layers were concentrated. The resultant residue was purified by flash chromatography (0-30% methanol/dichloromethane) to provide Compound 18d.

D. 1-(4-Difluoromethoxy-benzyl)-3-(4-fluoro-benzyl)-6-methylsulfanyl-1H-[1,3,5]triazine-2,4-dione (Cpd 18e). To a reaction vessel containing compound 18d (31 mg, 0.1 mmol) in acetonitrile (0.5 mL) was added cesium carbonate (32 mg, 0.1 mmol) and 4-fluorobenzyl bromide (18.9 mg, 0.1 mmol). The mixture was stirred at room temperature for 18 h, then concentrated. The residue was taken up in dichloromethane/water, absorbed onto diatomaceous earth, and eluted with dichloromethane. The eluate was concentrated to provide Compound 18e.

E. 1-(4-Difluoromethoxy-benzyl)-6-[(4,6-dimethyl-pyridin-3-ylmethyl)-amino]-3-(4-fluoro-benzyl)-1H-[1,3,5]triazine-2,4-dione (Cpd 160) To compound 18e in ethanol (0.5 mL) was added compound 2a (16 mg, 0.12 mmol). The mixture was irradiated at 180° C. in a microwave instrument for two 30 min intervals, then concentrated. The residue was dissolved in methyl sulfoxide and purified by reversed-phase chromatography to furnish the title compound 160 as its trifluoroacetate salt. ¹H NMR (methanol-d₄): δ 8.49 (s, 1H), 7.64 (s, 1H), 7.41 (m, 2H), 7.23 (d, 2H, J=8.7 Hz), 7.12 (d, 2H, J=8.6 Hz), 7.00 (t, 2H, J=8.8 Hz), 6.82 (t, 1H, ²J_{HF}=73.8 Hz), 5.19 (s, 2H), 4.99 (s, 2H), 4.61 (s, 2H), 2.67 (s, 3H), 2.38 (s, 3H); HRMS m/z (M+H)⁺ calcd for C₂₆H₂₅F₃N₅O₃ 512.1909, found 512.1911.

Other compounds of the present invention may be prepared by those skilled in the art by varying the starting materials, reagent(s) and conditions used. Using the general procedure of Example 18, the following compounds were prepared:

| Cpd | MS obs | MS calc |
|---|---|---|
| 85 | 545.8 | 546.5 |
| 158 | 560.3 | 560.6 |
| 159 | 620.2 | 620.4 |
| 161 | 508.2 | 508.5 |
| 162 | 562.1 | 562.5 |
| 163 | 560.1 | 560.5 |
| 164 | 519.2 | 519.5 |
| 165 | 552.2 | 552.6 |
| 166 | 524.5 | 524.5 |
| 167 | 542.5 | 542.5 |
| 168 | 578.2 | 578.6 |
| 173 | 555.2 | 555.6 |
| 174 | 565.2 | 565.6 |
| 175 | 549.2 | 549.6 |
| 176 | 551.2 | 551.6 |
| 177 | 540.2 | 540.6 |
| 178 | 534.2 | 534.5 |
| 179 | 536.3 | 536.6 |
| 180 | 519.2 | 519.5 |
| 182 | 552.2 | 552.6 |
| 185 | 527.2 | 527.6 |
| 186 | 525.1 | 525.6 |
| 191 | 524.2 | 524.5 |
| 192 | 549.2 | 549.6 |
| 193 | 524.3 | 524.5 |
| 194 | 537.4 | 537.5 |
| 195 | 560.3 | 560.5 |
| 196 | 552.2 | 552.6 |
| 198 | 504.4 | 504.6 |
| 208 | 538.1 | 538.5 |
| 210 | 552.2 | 552.6 |
| 219 | 553.1 | 553.5 |
| 221 | 564.2 | 564.6 |
| 227 | 533.2 | 533.6 |
| 228 | 520.0 | 520.5 |
| 242 | 515.1 | 514.57 |
| 243 | 528.13 | 527.61 |
| 244 | 512.36 | 511.55 |
| 245 | 525.23 | 524.58 |
| 268 | 512.22 | 511.49 |

### Additional ¹H NMR Data for Compounds of Example 18

6-[(2-Amino-pyridin-3-ylmethyl)-amino]-1-(4-difluoromethoxy-benzyl)-3-(4-methoxy-benzyl)-1H-[1,3,5]triazine-2,4-dione (Cpd 35). ¹H NMR (DMSO, d₆) δ 3.65 (s, 3H), 4.27 (d, 2H, J=5.03 Hz), 4.76 (s, 2H), 5.04 (s, 2H), 6.80 (m, 4H), 7.16 (m, 4H), 7.27 (d, 2H, J=8.72 Hz), 7.83 (d, 1H, J=6.07 Hz), 8.18 (m, 1H).

6-[(2-Amino-4,6-dimethyl-pyridin-3-ylmethyl)-amino]-1,3-bis-(2,3-dihydro-benzofuran-5-ylmethyl)-1H-[1,3,5]triazine-2,4-dione (Cpd 185). ¹H NMR (DMSO, d₆) δ 2.36 (s, 3H), 2.37 (s, 3H), 3.10 (td, 4H, J=5.72, 3.59 Hz), 4.36 (m, 2H), 4.49 (td, 4H, J=5.05, 3.55 Hz), 4.81 (s, 2H), 5.00 (s, 2H), 6.65 (s, 1H), 6.68 (d, 2H, J=8.19 Hz), 7.01 (m, 4H), 7.50 (s, 1H), 8.01 (s, 1H).

### Example 19

### C-Imidazo[1,2-a]pyridin-8-yl-methylamine (Cpd 17c)

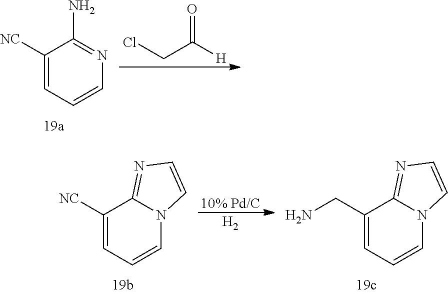

A. Imidazo[1,2-a]pyridine-8-carbonitrile (Cpd 19b). To a solution of 2-amino-3-cyanopyridine (Cpd 19a) (1.0 g, 8.4 mmol) in ethanol (20 mL) was added chloroacetaldehyde (1.57 g, 50 wt. % solution in water, 10.0 mmol). The mixture was irradiated at 120° C. in a microwave instrument for 30 min. After quenching with saturated aqueous sodium carbonate, the mixture was concentrated. The residue was taken up in dichloromethane/water and the layers were separated. The aqueous layer was extracted with dichloromethane (2×) and the combined organic layer was washed with brine, dried over MgSO₄, filtered, and the filtrate was concentrated to provide compound 19b.

B. C-Imidazo[1,2-a]pyridin-8-yl-methylamine (Cpd 19c). A mixture of compound 19b (413 mg, 2.88 mmol), palladium (100 mg, 10 wt. % support activated carbon), and ammonia (40 mL, 2M solution in methanol) was hydrogenated at 55 psi pressure for 18 h at room temperature. The reaction mixture was filtered through a pad of diatomaceous earth and washed with methanol. The filtrate was concentrated to provide compound 19c, which was used in subsequent reactions without further purification.

### Example 20

### 6-[(Imidazo[1,2-a]pyridin-8-ylmethyl)-amino]-1,3-bis-(4-methoxy-benzyl)-1H-[1,3,5]triazine-2,4-dione (Cpd 188)

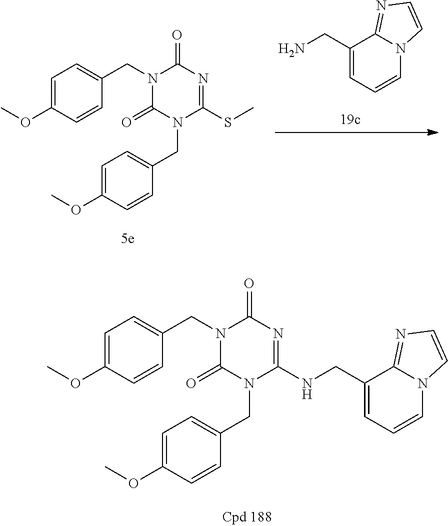

A solution of compound 5e (60 mg, 0.15 mmol) and compound 19c (26 mg, 0.18 mmol) in ethanol (0.5 mL) was irradiated at 180° C. in a microwave instrument for two 30 min intervals, then concentrated. The residue was dissolved in methyl sulfoxide and purified by reversed-phase chromatography to furnish the title compound 188 as its trifluoroacetate salt. ¹H NMR (methanol-d₄): δ 8.66 (d, 1H, J=6.8 Hz), 8.20 (d, 1H, J=2.2 Hz), 8.01 (d, 1H, J=2.2 Hz), 7.46 (d, 1H, J=7.4 Hz), 7.33 (d, 2H, J=8.6 Hz), 7.28 (t, 1H, J=7.0 Hz), 7.15 (d, 2H, J=8.6 Hz), 6.88 (d, 2H, J=8.8 Hz), 6.83 (d, 2H, J=8.8 Hz), 5.15 (s, 2H), 4.96 (s, 2H), 4.88 (s, 2H), 3.78 (s, 3H), 3.75 (s, 3H); HRMS m/z (M+H)⁺ calcd for C₂₇H₂₇N₆O₄ 499.2094, found 499.2052.

### Example 21

### 3-Ethynyl-2-nitro-pyridine (Cpd 21c)

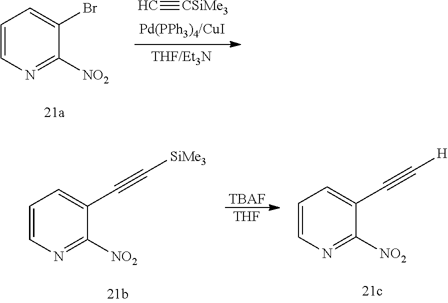

A. 2-Nitro-3-trimethylsilanylethynyl-pyridine (Cpd 21b). Compound 21a (500 mg, 2.5 mmol) and TMS-acetylene (500 μL) were dissolved in a mixture of dry THF/triethylamine (10 mL/2 mL) under a nitrogen atmosphere. Pd(PPh₃)₄ (70 mg) was added as one portion, followed by of copper (I) iodide (50 mg). The stirred solution was kept overnight at RT and evaporated. The residue was subjected to normal phase column chromatography (silica gel, heptane/EtOAc 2:1), providing compound 21b. ¹H NMR (CDCl₃) δ 0.27 (s, 9H), 7.57 (dd, 1H, J=7.83 and 4.69 Hz), 8.06 (dd, 1H, J=7.86 and 1.70 Hz), 8.48 (dd, 1H, J=4.66 and 1.69 Hz).

B. 3-Ethynyl-2-nitro pyridine (Cpd 21c) Compound 21b was dissolved in dry THF (10 mL) at RT and 1 M TBAF in THF (1 mL) was added dropwise over 10 min. The reaction mixture was kept at RT for 1 h, evaporated, dissolved in EtOAc/heptane (1/1 mixture) and filtered through a silica gel plug. After evaporation, compound 21c was obtained and used in the next step without further purification.

### Example 22

### 6-[2-(2-Amino-pyridin-3-yl)-ethyl]-1,3-bis-(4-methoxy-benzyl)-1H-pyrimidine-2,4-dione (Cpd 199)

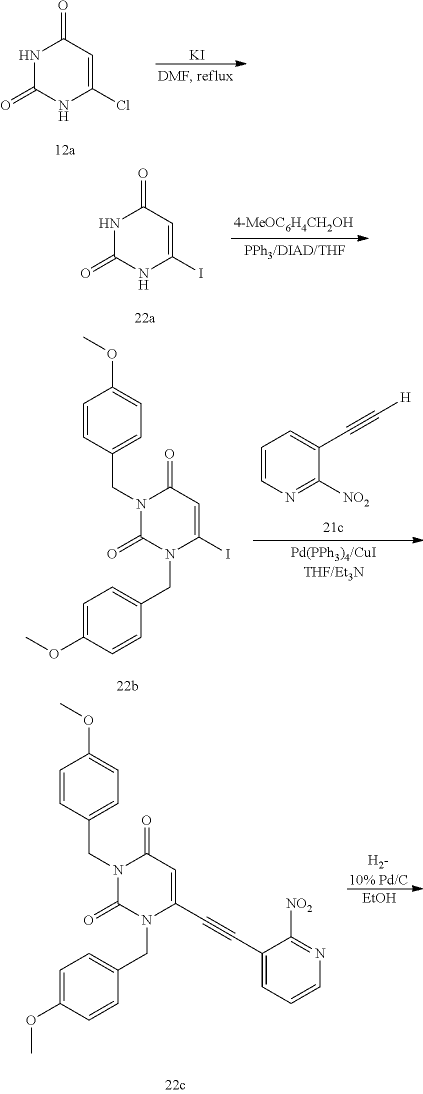

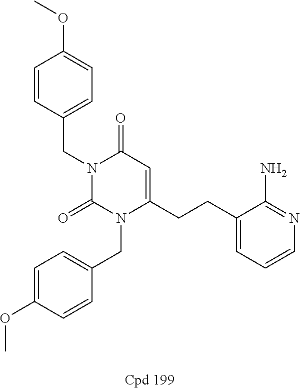

A. 6-Iodo-1H-pyrimidine-2,4-dione (Cpd 22a) Compound 12a (5 g, 34 mmol) and sodium iodide (20 g) were dissolved in anhydrous DMF (50 mL) and heated to reflux for 1.5 h (Ar atmosphere). The DMF was evaporated, and the solid residue dissolved in H₂O (200 mL). The solution was stirred at RT for 4 h, a solid material was collected by vacuum filtration, and the solid was washed with H₂O and dried. The solid was crystallized from EtOAc, providing compound 22a. ¹H NMR (DMSO-d₆) δ 6.03 (s, 1H), 11.2 (s, 1H), 11.6 (s, 1H).

B. 6-Iodo-1,3-bis-(4-methoxy-benzyl)-1H-pyrimidine-2,4-dione (Cpd 22b). Compound 22a (1.00 g, 4.2 mmol), 4-methoxybenzyl alcohol (1.7 g, 3 eq), PPh₃ (4.00 g) were dissolved in dry THF (25 mL) under an atmosphere of N₂. DIAD was added dropwise at approximately 1 mL/min until the yellow color remained (about 4 eq total). The reaction mixture was stirred for 4 h at RT and evaporated. The residue was subjected to normal phase column chromatography (silica gel, gradient mixture heptane-ethyl acetate), providing compound 22b. ¹H NMR (CDCl₃) δ 3.78 (s, 3H), 3.79 (s, 3H), 5.04 (s, 2H), 5.27 (s, 2H), 6.54 (s, 1H), 6.82 (d, J=7.3 Hz, 2H), 6.86 (d, J=8.7 Hz, 2H), 7.22 (d, J=7.3 Hz, 2H), 7.42 (d, J=8.7 Hz, 2H). MS m/z (ES) 479.1 (M+H).

C. 1,3-Bis-(4-methoxy-benzyl)-6-(2-nitro-pyridin-3-ylethynyl)-1H-pyrimidine-2,4-dione (Cpd 22c) Compound 22b (240 mg, 0.5 mmol) and compound 21c (150 mg, 1 mmol) were dissolved in a mixture of dry THF (10 mL) and Et₃N (2 mL). Pd(PPh₃)₄ (40 mg) and copper (I) iodide (20 mg) were added simultaneously in one portion. The reaction mixture was stirred overnight at RT under a N₂ atmosphere and evaporated. The residue was subjected to normal phase column chromatography (silica gel column, EtOAc), providing compound 22c. ¹H NMR (CDCl₃) δ 3.76 (s, 3H), 3.78 (s, 3H), 5.06 (s, 2H), 5.23 (s, 2H), 6.17 (s, 1H), 6.82 (d, J=8.6 Hz), 7.27 (d, J=6.4 Hz, 2H), 7.44 (dd, J=6.7 and 2.02 Hz, 2H), 7.68 (dd, J=7.8 and 4.6 Hz, 1H), 8.06 (dd, J=7.8 and 1.7 Hz, 1H), 8.63 (dd, J=4.7 and 1.7 Hz, 1H).

D. 6-[2-(2-Amino-pyridin-3-yl)-ethyl]-1,3-bis-(4-methoxy-benzyl)-1H-pyrimidine-2,4-dione (Cpd 199). Compound 22c (100 mg, 0.2 mmol) was dissolved in EtOH (10 mL) and suspended with 10% Pd on carbon (40 mg). The reaction mixture was hydrogenated for 24 h at RT under atmospheric pressure, filtered through a Diatomaceous earth plug, and evaporated. The residual material was purified by reverse phase HPLC chromatography (water/acetonitrile gradient), and then lyophilized, to provide compound 199. ¹H NMR (DMSO-d₆) δ 2.8 (m, 4H), 3.43 (s, 6H), 4.96 (s, 2H), 5.11 (s, 2H), 5.82 (s, 1H), 6.88 (m, 4H), 7.15 (m, 2H), 7.24 (m, 2H), 7.77 (m, 1H), 7.86 (m, 1H), 7.92 (m, 1H). MS m/z (ES) 473.2 (M+H).

Using an adaptation of the methods described in Example 22, compound 169 was prepared from compound 22i, substituting 3-ethynyl pyridine for compound 21c of Example 22, Step C.

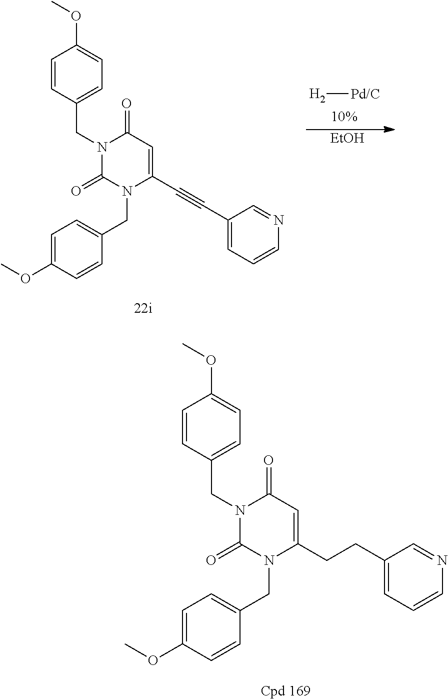

Cpd 22i: ¹H NMR (DMSO-d₆) δ 3.71 (s, 3H), 3.72 (s, 3H), 4.95 (s, 2H), 5.19 (s, 2H), 6.27 (s, 1H), 6.87 (d, J=8.3 Hz, 2H), 6.89 (d, J=7.7 Hz, 2H), 7.28 (m, 4H), 7.52 (m, 1H), 8.1 (m, 1H), 8.8 (m, 2H).

Cpd 169: ¹H NMR (DMSO-d₆) δ 2.88 (m, 2H), 2.95 (m, 2H), 3.72 (s, 6H), 4.94 (s, 2H), 5.11 (s, 2H), 5.72 (s, 1H), 6.87 (d, J=8.6 Hz, 2H), 6.89 (d, J=7.6 Hz, 2H), 7.11 (d, J=8.6 Hz, 2H), 7.22 (d, J=7.8 Hz, 2H), 7.79 (m, 1H), 8.20 (m, 1H), 8.71 (m, 2H).

Using an adaptation of the methods described in Example 22, compound 187 was prepared from compound 22k, substituting 2-ethynyl pyridine for compound 21c of Example 22, Step C.

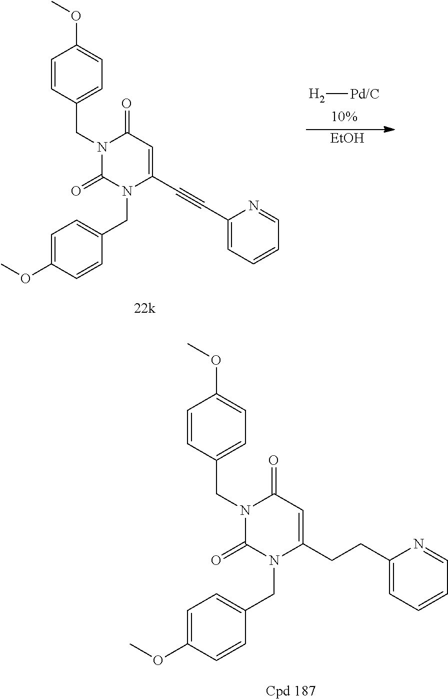

Cpd 22k: ¹H NMR (DMSO-d₆) δ 3.71 (s, 3H), 3.72 (s, 3H), 4.95 (s, 2H), 5.17 (s, 2H), 6.29 (s, 1H), 6.89 (m, 4H), 7.26 (d, J=8.6 Hz, 2H), 7.32 (d, J=8.6 Hz, 2H), 7.54 (m, 1H), 7.72 (d, J=7.8 Hz, 1H), 7.92 (m, 1H), 8.7 (m, 1H).

Cpd 187: ¹H NMR (DMSO-d₆) δ 2.92 (m, 2H), 3.10 (m, 2H), 3.72 (s, 6H), 4.93 (s, 2H), 5.10 (s, 2H), 5.66 (s, 1H), 6.88 (m, 4H), 7.11 (d, J=8.6 Hz, 2H), 7.22 (d, J=8.7 Hz, 2H), 7.50 (m, 2H), 8.01 (m, 1H), 8.61 (d, J=4.49 Hz, 1H).

Other compounds of the present invention may be prepared by those skilled in the art by varying the starting materials, reagent(s) and conditions used. Using the general procedure of Example 22, the following compounds were prepared:

| Cpd | MS obs | MS calc |
|---|---|---|
| 169 | 458.0 | 458.5 |
| 183 | 457.9 | 458.5 |
| 187 | 458.1 | 458.5 |
| 189 | 500.9 | 501.6 |
| 199 | 473.2 | 473.5 |
| 214 | 472.8 | 473.5 |

### Example 23

### 6-[(2-Amino-4,6-dimethyl-1-oxy-pyridin-3-ylmethyl)-amino]-1-(4-difluoromethoxy-benzyl)-3-(2,3-dihydro-benzofuran-5-ylmethyl)-1H-[1,3,5]triazine-2,4-dione (Cpd 233)

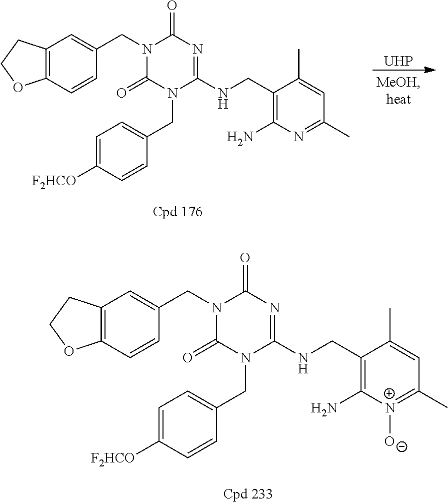

A. Compound 176 (50 mg, 0.09 mmol) was prepared from compound 18d using the method described in Example 5, substituting 2,3-dihydrobenzofuran-5-yl methanol for 4-methoxybenzyl alcohol in Step E; and substituting 2-amino-3-aminomethyl-4,6-dimethylpyridine for Compound 2a in Step F.

B. 6-[(2-Amino-4,6-dimethyl-1-oxy-pyridin-3-ylmethyl)-amino]-1-(4-difluoromethoxy-benzyl)-3-(2,3-dihydro-benzofuran-5-ylmethyl)-1H-[1,3,5]triazine-2,4-dione (Cpd 233). Compound 176 and urea-hydrogen peroxide addition complex (200 mg) were combined and the mixture was heated to 85° C. After 4 hours, the mixture was dissolved in methanol (3 mL) and the temperature was reduced to 70° C. After stirring overnight, the mixture was allowed to cool and was poured over H₂O (15 mL). The reaction was diluted with water, extracted with ethyl acetate (3×10 mL) and the combined extracts were dried over Na₂SO₄, filtered and reduced. Purification by reverse-phase prep HPLC afforded Cpd 233. MS m/z (ES)=566.8 (M+H); ¹H NMR (DMSO, d₆) δ 2.29 (s, 3H), 2.38 (s, 3H), 3.11 (t, 2H, J=8.49 Hz), 4.40 (m, 2H), 4.48 (t, 2H, J=8.72 Hz), 4.80 (s, 2H), 5.04 (s, 2H), 6.68 (d, 2H, J=4.64 Hz), 7.15 (m, 4H), 7.20 (s, 1H), 7.25 (d, 2H, J=8.57 Hz).

### Example 24

### 6-[(2-Amino-4,6-dimethyl-1-oxy-pyridin-3-ylmethyl)-amino]-1,3-bis-(2,3-dihydro-benzofuran-5-ylmethyl)-1H-[1,3,5]triazine-2,4-dione (Cpd 226)

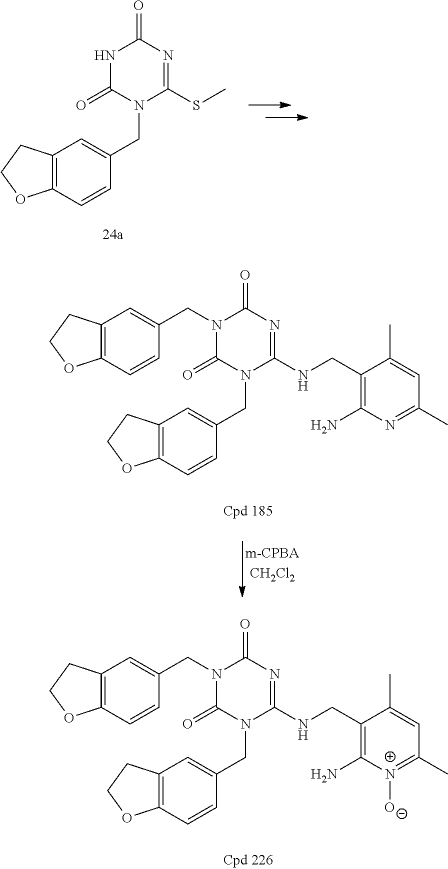

A. Compound 24a was prepared by the methods described in Example 18, Steps A through C, substituting 2,3-dihydrobenzofuran-5-yl methyl amine for 4-difluoromethoxybenzyl amine in Step A.

B. Compound 185 (40 mg, 0.08 mmol) was prepared from compound 24a using the method described in Example 5, substituting 2,3-dihydrobenzofuran-5-yl methanol for 4-methoxybenzyl alcohol in Step E; and substituting 2-amino-3-aminomethyl-4,6-dimethylpyridine for Compound 2a in Step F.

C. 6-[(2-Amino-4,6-dimethyl-1-oxy-pyridin-3-ylmethyl)-amino]-1,3-bis-(2,3-dihydro-benzofuran-5-ylmethyl)-1H-[1,3,5]triazine-2,4-dione (Cpd 226). A solution of compound 185 in dichloromethane (4 mL) was treated with m-CPBA (72%, 30 mg, 0.15 mmol) and the mixture was stirred overnight at room temperature. The reaction was then poured over 10% Na₂S₂O₄ and the organic phase was extracted with CH₂Cl₂ (3×10 mL). The combined organic layers were then washed with saturated NaHCO₃ (3×10 mL) and were again extracted with dichloromethane (3×5 mL). The organic extracts were then combined and dried over Na₂SO₄, filtered, and reduced. Purification via reverse phase HPLC afforded Cpd 226 as its TFA salt. The resulting TFA salt was taken up in dichloromethane (5 mL) and was washed with saturated NaHCO₃ (3×5 mL). Combined organic extracts were dried over Na₂SO₄, filtered and reduced to afford Compound 226 as its free-base. M⁺ (ES⁺)=543.34.

Other compounds of the present invention may be prepared by those skilled in the art by varying the starting materials, reagent(s) and conditions used. Using the general procedure of Example 24, the following compounds were prepared:

| Cpd | MS obs | MS calc |
|---|---|---|
| 32 | 491.2 | 491.5 |
| 53 | 476.2 | 476.5 |
| 118 | 504.2 | 504.6 |
| 269 | 488.19 | 487.52 |

### Example 25

### 6-[2-(6-Amino-pyridin-2-yl)-ethyl]-1,3-bis-(4-methoxy-benzyl)-1H-pyrimidine-2,4-dione (Cpd 223)

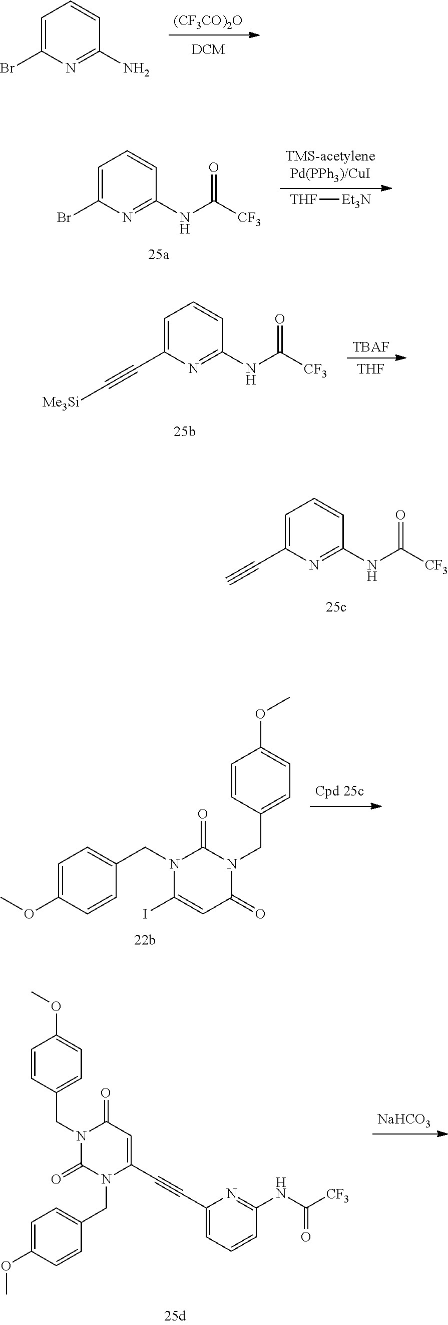

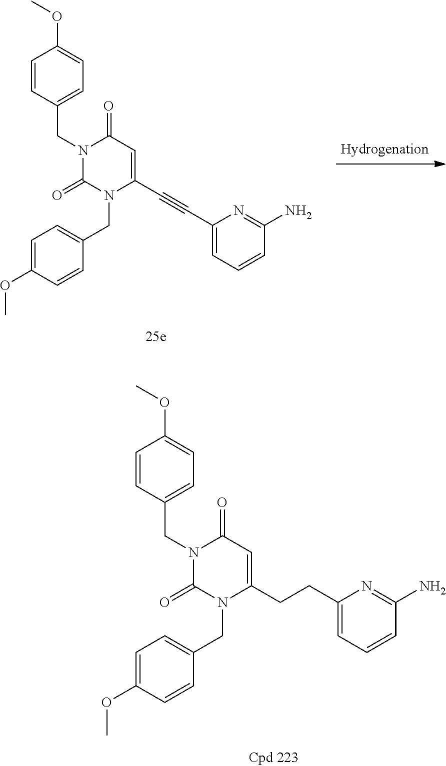

A. 6-Bromo-2-trifluoroacetamido-pyridine (Cpd 25a). 2-Amino-6-bromopyridine (800 mg) was dissolved in a mixture of DCM (30 mL) and TEA (2 mL), and the solution was cooled in an ice bath. Trifluoroacetic anhydride (2 mL) was added by 100 μL portions. The reaction mixture was allowed to warm up to room temperature, and then was washed sequentially with water and 10% sodium bicarbonate solution. The mixture was dried, filtered, and the filtrate was evaporated. The residue was subjected to normal phase column chromatography (silica gel, heptane/ethyl acetate 1:1), providing compound 25a. ¹H NMR (CDCl₃) δ 8.65 (broad s, 1H), 8.15 (d. J=8.2 Hz, 1H), 7.67 (t, J=7.9 Hz, 1H), 7.37 (d, J=8.1 Hz, 1H).

B. 2,2,2-Trifluoro-N-(6-trimethylsilanylethynyl-pyridin-2-yl)-acetamide (Cpd 25b) Compound 25b was prepared using the methods described in Example 21, Step A. ¹H NMR (CDCl₃) δ 8.57 (broad s, 1H), 7.96 (d, J=8.3 Hz, 1H), 7.57 (t, J=8.0 Hz, 1H), 7.15 (d, J=8.3 Hz, 1H), 0.09 (s, 9H).

C. N-(6-Ethynyl-pyridin-2-yl)-2,2,2-trifluoro-acetamide (Cpd 25c). Compound 25c was prepared using the methods described in Example 21, Step B, substituting compound 25b for compound 21b. Purification was achieved by normal phase column chromatography (silica gel, heptane/ethyl acetate 2:1). ¹H NMR (CDCl₃) δ 8.62 (broad s, 1H), 8.20 (d, J=8.3 Hz, 1H), 7.80 (t, J=8.0 Hz, 1H), 7.38 (d, J=8.3 Hz, 1H), 3.21 (s, 1H).

D. N-{6-[1,3-Bis-(4-methoxy-benzyl)-2,6-dioxo-1,2,3,6-tetrahydro-pyrimidin-4-ylethynyl]-pyridin-2-yl}-2,2,2-trifluoro-acetamide (Cpd 25d). Compound 25d was prepared using the methods described in Example 22, Step C, substituting compound 25c for compound 21c. Purification was achieved by reverse phase HPLC. MS m/z 565.2 (M+H).

E. 6-(6-Amino-pyridin-2-ylethynyl)-1,3-bis-(4-methoxy-benzyl)-1H-pyrimidine-2,4-dione (Cpd 25e). Compound 25d (550 mg) was dissolved in EtOH (5 mL), and a saturated solution of NaHCO₃ (5 mL) was added. After stirring for 1 h at room temperature, the reaction mixture was concentrated under reduced pressure, and the resultant residue was subjected to reverse phase HPLC and subsequent lyophilization to afford compound 25e.

F. 6-[2-(6-Amino-pyridin-2-yl)-ethyl]-1,3-bis-(4-methoxy-benzyl)-1H-pyrimidine-2,4-dione (Cpd 223). Compound 223 was prepared using the methods described in Example 22, Step D, substituting compound 25e for compound 22c. Purification was achieved by reverse phase HPLC followed by lyophilization. MS m/z (ES) 470.9 (M+H).

### Example 26

### 1,3-Bis-(4-methoxy-benzyl)-6-(2-pyridin-4-yl-vinyl)-1H-pyrimidine-2,4-dione (Cpd 184)

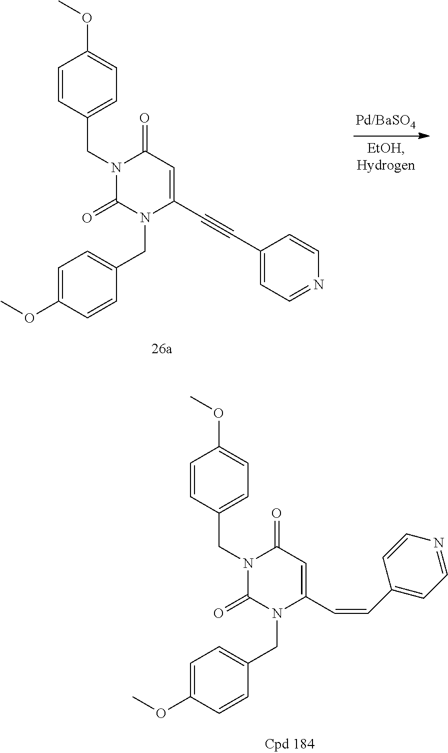

Compound 26a was prepared using the methods described in Example 22, Step C, substituting 4-ethynylpyridine for compound 21c. Compound 26a (100 mg, TFA salt) was suspended with Pd on BaSO₄ (5%, 40 mg) in EtOH (20 mL). The reaction mixture was hydrogenated for 3 h at RT and atmospheric pressure, filtered through a pad of diatomaceous earth and concentrated under reduced pressure. The residual material was purified by HPLC, followed by lyophilization to give compound 184. MS m/z (ES) 455.9 (M+H).

### Example 27

### 6-[(2-Amino-pyridin-3-ylmethyl)-amino]-1-(4-hydroxy-benzyl)-3-(4-methoxy-benzyl)-1H-[1,3,5]triazine-2,4-dione (Cpd 33)

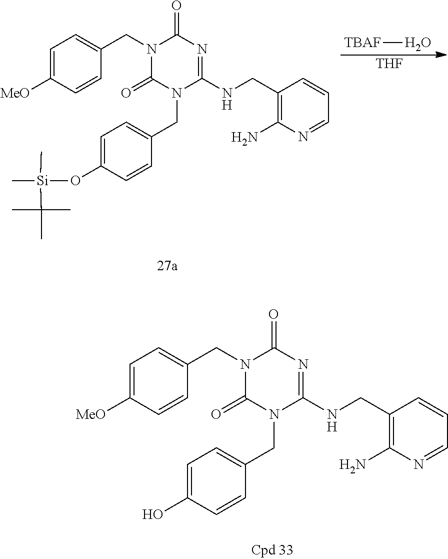

A. Compound 27a (80 mg, 0.14 mmol) was prepared according to the methods described in Example 2, and substituting [4-(tert-butyl-dimethyl-silanyloxy)-phenyl]-methanol for 4-methoxybenzyl alcohol in Step D.

B. 6-[(2-Amino-pyridin-3-ylmethyl)-amino]-1-(4-hydroxy-benzyl)-3-(4-methoxy-benzyl)-1H-[1,3,5]triazine-2,4-dione (Cpd 33). Compound 27a was suspended in THF (3 mL) and the reaction mixture was treated with tetrabutylammonium fluoride monohydrate (36 mg, 0.14 mmol). The solution was stirred at room temperature overnight. The mixture was then concentrated under nitrogen and the residue was purified by reverse phase HPLC to give the title compound 33. MS m/z (ES)=461.2 (M+H); ¹H NMR (DMSO, d₆) δ 3.72 (s, 3H), 4.33 (m, 2H), 4.83 (s, 2H), 5.01 (s, 2H), 6.75 (m, 3H), 6.84 (d, 2H, J=8.71 Hz), 7.08 (d, 2H, J=8.56 Hz), 7.24 (d, 2H, J=8.63 Hz), 7.46 (d, 1H, J=8.06 Hz), 7.89 (d, 1H, J=4.88 Hz).

### Example 28

### 6-{[(2-Amino-pyridin-3-ylmethyl)-amino]-methyl}-1,3-bis-(4-methoxy-benzyl)-1H-pyrimidine-2,4-dione (Cpd 7)

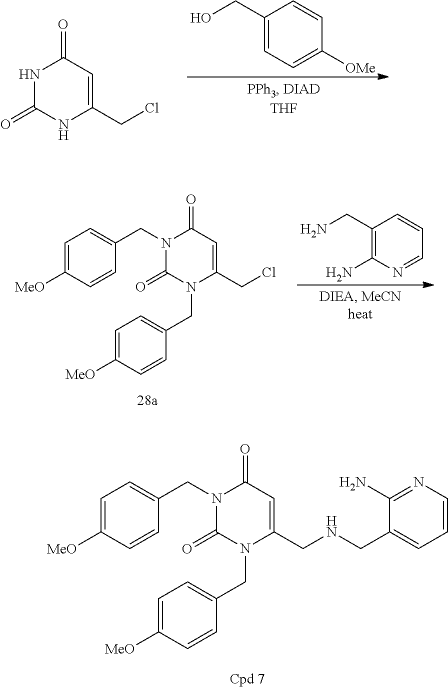

A. 6-Chloromethyl-1,3-bis-(4-methoxy-benzyl)-1H-pyrimidine-2,4-dione (Cpd 28a). 6-Chloromethyl uracil (500 mg, 3.1 mmol) was dissolved in THF (50 mL) and the solution was treated with 4-methoxybenzyl alcohol (860 mg, 6.2 mmol), triphenylphosphine (2.45 g, 9.3 mmol) and diisopropylazodicarboxylate (1.26 g, 6.2 mmol). The reaction was allowed to stir overnight at room temperature. The mixture was then poured over water (75 mL) and was extracted with ethyl acetate (3×50 mL). The combined organic extracts were dried over Na₂SO₄, filtered and reduced. Compound 28a was isolated and purified by normal phase column chromatograpy (silica gel, 20% EtOAc/heptane-100% EtOAc/heptane). M⁺ (ES⁺)=401.1.

B. 6-{[(2-Amino-pyridin-3-ylmethyl)-amino]-methyl}-1,3-bis-(4-methoxy-benzyl)-1H-pyrimidine-2,4-dione (Cpd 7). Cpd 28a (100 mg, 0.25 mmol) was dissolved in acetonitrile (5 mL) and the reaction mixture was treated with diisopropylethylamine (0.087 mL, 0.50 mmol), and 2-amino-3-methylaminopyridine (Cpd 1a) (31 mg, 0.25 mmol). The solution was heated to 80° C. and was allowed to stir for 4 hours. The mixture was then cooled to room temperature and was poured over saturated NH₄Cl (15 mL). The desired product was extracted with ethyl acetate (3×10 mL) and the combined organic extracts were dried over Na₂SO₄, filtered and reduced. Purification and isolation by reverse phase HPLC gave compound 7. MS m/z (ES)=488.1 (M+H); ¹H NMR (DMSO, d₆) δ 2.83 (s, 2H), 3.02 (s, 2H), 4.07 (s, 6H), 4.26 (s, 2H), 4.34 (s, 2H), 5.24 (s, 1H), 6.05 (m, 5H), 6.20 (d, 2H, J=6.99 Hz), 6.54 (d, 2H, J=7.05 Hz), 6.92 (t, 2H, J=7.71 Hz).

### Example 29

### 6-[(2-Amino-pyridin-3-ylmethyl)-amino]-1,3-bis-(4-methoxy-benzyl)-1H-[1,3,5]triazine-2,4-dione (Cpd 3)

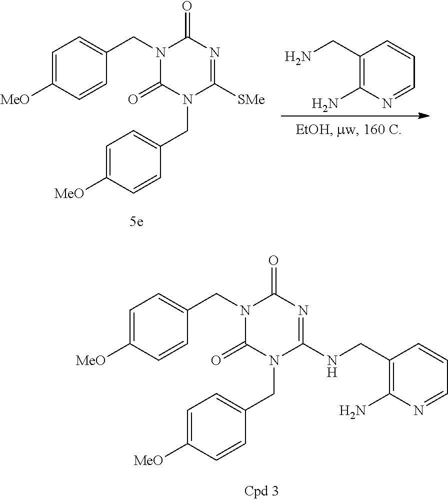

Cpd 5e (850 mg, 2.1 mmol) and Cpd 1a (524 mg, 4.3 mmol) were suspended in ethanol (10 mL) and the reaction mixture was irradiated at 160° C. for 100 minutes in a microwave instrument. The solution was reduced in vacuo and purified by reverse phase HPLC to afford the title compound 3. MS m/z (ES)=475.2 (M+H), ¹H NMR (DMSO, d₆) δ 3.71 (s, 3H), 3.74 (s, 3H), 4.36 (d, 2H, J=4.59 Hz), 4.83 (s, 2H), 5.09 (s, 2H), 6.90 (m, 4H), 7.24 (d, 4H, J=8.64 Hz), 7.57 (d, 1H, J=7.08 Hz), 7.91 (d, 1H, J=6.39 Hz), 8.08 (s, 2H), 8.45 (m, 1H).

### Example 30

### Pyridin-3-yl-methanthiol (Cpd 30a)

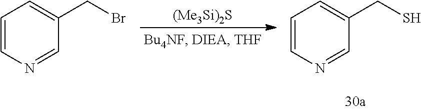

Pyridin-3-yl-methanthiol (Cpd 30a). To a mixture of 3-(bromomethyl)pyridine hydrobromide (500 mg, 2.0 mmol) and diisopropylethylamine (0.220 mL, 2.0 mmol) in THF (20 mL), cooled in a sodium chloride/ice bath (−5° C.), was added hexamethyldisilathiane (0.500 mL, 2.4 mmol) and tetrabutylammonium fluoride (575 mg, 2.2 mmol). The resulting mixture was allowed to warm to room temperature and stirred overnight. The mixture was then concentrated and the residue partitioned between ethyl acetate and saturated aqueous ammonium chloride. The organic layer was separated, dried over MgSO₄ and concentrated. The concentrate was purified by normal phase chromatography, eluting with ethyl acetate to obtain compound 30a. ¹H NMR (MeOD, d₄) δ3.77 (s, 2H), 7.38-7.41 (m, 1H), 7.84-7.86 (d, 1H, J=7.96), 8.38-8.40 (m, 1H), 8.50 (s, 1H).

### Example 31

### 1,3-Bis-(4-methoxy-benzyl)-6-(pyridin-3-ylmethylsulfanyl)-1H-pyrimidine-2,4-dione (Cpd 211)

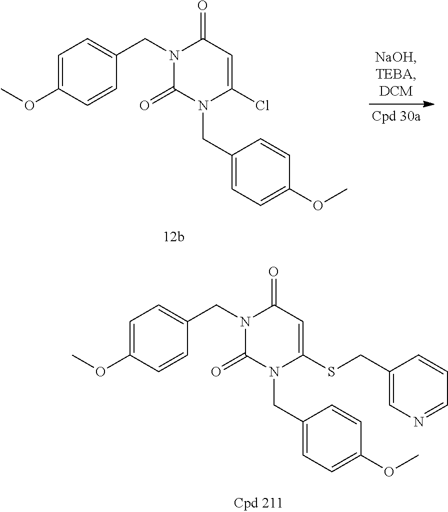

A solution of Compound 12b (97 mg, 0.25 mmol), Compound 30a (61 mg, 0.49 mmol), NaOH (3M, 1.67 mL, 5 mmol), and TEBA (6 mg, 0.025 mmol) in 2 mL of dichloromethane, was stirred vigorously overnight at room temperature. After 24 hours, an additional amount of Compound 12b was added (50 mg) and the mixture allowed to stir for a second night. The mixture was then separated, the organic layer was dried over MgSO₄, filtered, and the filtrate was concentrated. The concentrate was purified by reverse phase chromatography to obtain compound 211. MS m/z (ES)=475.8 (M+H). ¹H NMR (DMSO, d₆). δ 3.72-3.73 (d, 6H, J=3.8 Hz), 4.47 (s, 2H), 4.91 (s, 2H), 5.07 (s, 2H), 5.85 (s, 1H), 6.84-6.89 (m, 4H), 7.12-7.15 (d, 2H, J=9.4 Hz), 7.21-7.23 (d, 2H, J=8.7 Hz), 7.57-7.61 (m, 1H), 8.03-8.06 (m, 1H), 8.61-8.63 (d, 1H, J=4.3 Hz), 8.73 (s, 1H).

### Example 32

### 6-[(2-Amino-4-benzyloxymethyl-6-methyl-pyridin-3-ylmethyl)-amino]-1-(4-difluoromethoxy-benzyl)-3-(2,3-dihydro-benzofuran-5-ylmethyl)-1H-[1,3,5]triazine-2,4-dione (Cpd 251)

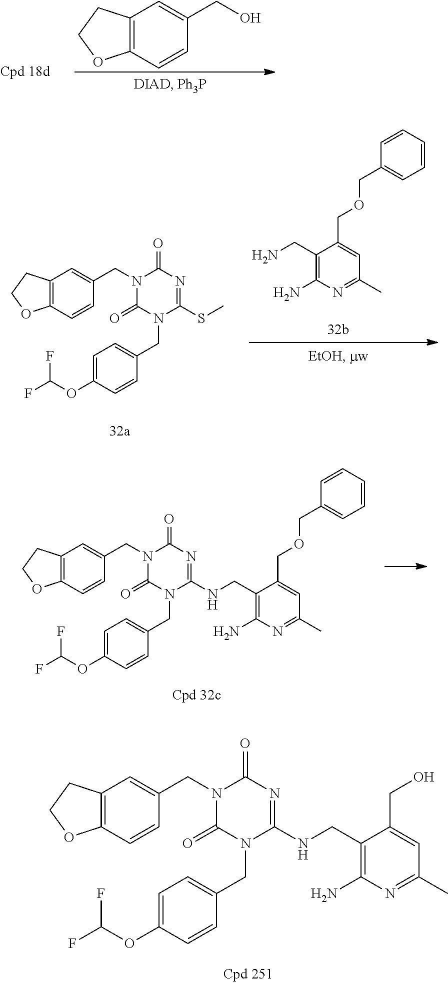

To compound 18d (2.8 g, 8.9 mmol) in 100 mL of THF was added DIAD (2.1 mL, 10.7 mmol), triphenyl phosphine (17.8 mmol), and 2,3-dihydro-1-benzofuran-5-ylmethanol. The mixture was allowed to stir at rt under an atmosphere of Argon. The mixture was concentrated, diluted with EtOAc, and washed with water. The organic phase was partitioned, dried over MgSO₄, filtered, and the filtrate was concentrated to a yellow oil. The oil was purified by reverse-phase chromatography to furnish compound 32a.

Compound 251 was prepared by an adaptation of the method described in Example 5, Step F, substituting Compound 32a for Compound 5e, and substituting Compound 32b for Compound 2a. Conventional removal of the benzyl protecting group gave compound 251.

Other compounds of the present invention may be prepared by those skilled in the art by varying the starting materials, reagent(s) and conditions used. Using the general procedure of Example 32, the following compounds were prepared:

| Cpd | MS obs | MS calc |
|---|---|---|
| 261 | 523.2 | 522.51 |
| 262 | 631.2 | 630.63 |

### Example 33

### 6-[(2-Amino-4,6-dimethyl-pyridin-3-ylmethyl)-amino]-1-(4-methoxy-benzyl)-3-(5-methoxy-pentyl)-1H-[1,3,5]triazine-2,4-dione (Cpd 252)

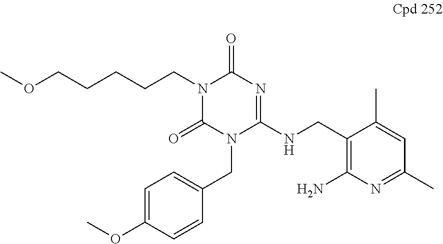

Compound 252 was prepared from Compound 8c using an adaptation of the methods described in Example 8, substituting 5-methoxy-pentan-1-ol for 2,3-dihydro-1-benzofuran-5-ylmethanol in Step C.

### Example 34

### 6-[(2-Amino-pyridin-3-ylmethyl)-amino]-1-(4-methoxy-benzyl)-3-(4-[1,2,3]thiadiazol-5-yl-benzyl)-1H-[1,3,5]triazine-2,4-dione (Cpd 240)

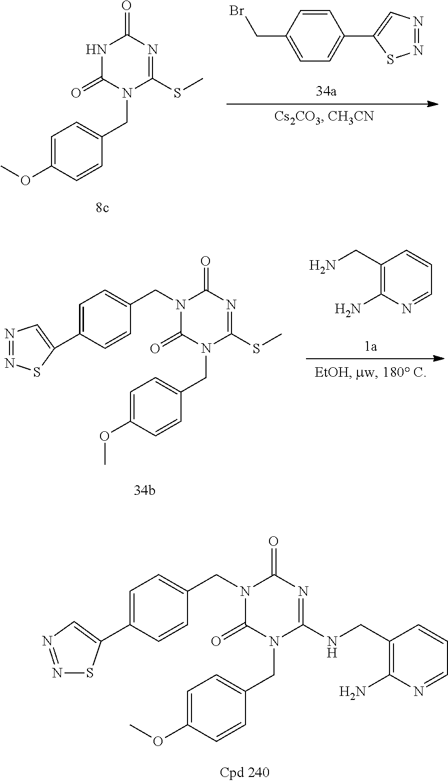

A. To Compound 8c (0.028 g, 0.1 mmol) in 0.5 mL CH₃CN was added cesium carbonate (0.032 g, 0.1 mmol) followed by the addition of Compound 34a (0.0255 g, 0.1 mmol) and the mixture was stirred at 25° C. for 16 h. At that time the mixture was concentrated. The resulting residue was partitioned between methylene chloride and water, and the organic phase was dried and concentrated to give Compound 34b.

B. Compound 34b was dissolved in ethanol (0.5 mL) and Compound 1a (0.018 mg, 0.15 mmol) was added. The mixture was irradiated at 180° C. for two 30 min cycles in a microwave instrument. The reaction was concentrated, the resultant residue was dissolved in DMSO, and the product was purified and isolated by reverse phase HPLC to afford Compound 240. MS m/z (ES)=529.17 (M+H), 528.59 calc'd.

Using the methods described in the schemes and specific examples, and adaptations thereof, compounds 1 to 272 of Table 1 were prepared.

TABLE 1

| Cpd No. | A₁ | L₁ | D | W | Q |
|---|---|---|---|---|---|
| 1 | 3,4-dichloro- | CH₂ | 4-methoxy- | N | 2-(pyridin-2-yl) |
| | phenyl | | phenylmethyl | | ethyl-amino |
| 2 | 3,4-dichloro- | CH₂ | 4-methoxy- | N | pyridin-3-yl |
| | phenyl | | phenylmethyl | | methyl amino |
| 3 | 4-methoxy- | CH₂ | 4-methoxy- | N | 2-amino- |
| | phenyl | | phenylmethyl | | pyridin-3-yl |
| | | | | | methyl-amino |
| 4 | 4-chloro- | CH₂ | 4-methoxy- | N | 5-amino- |
| | phenyl | | phenylmethyl | | pyridin-2-yl |
| | | | | | methyl-amino |
| 5 | 4-chloro- | CH₂ | 4-methoxy- | N | 6-amino- |
| | phenyl | | phenylmethyl | | pyridin-3-yl |
| | | | | | methyl-amino |
| 6 | 4-methoxy- | CH₂ | 4-methoxy- | N | 4-amino-pyrimidin-5-yl |
| | phenyl | | phenylmethyl | | methyl-amino |
| 7 | 4-methoxy- | CH₂ | 4-methoxy- | CH | 2-amino-pyridin-3- |
| | phenyl | | phenylmethyl | | ylmethyl-aminomethyl |
| 8 | 4-fluoro- | CH₂ | 4-methoxy- | N | 2-amino- |
| | phenyl | | phenylmethyl | | pyridin-3-ylmethyl- |
| | | | | | amino |
| 9 | 4-methoxy- | CH₂ | 4-methoxy- | N | 2-amino-quinolin-3- |
| | phenyl | | phenylmethyl | | ylmethyl-amino |
| 10 | 4-fluoro- | CH₂ | 4-methoxy- | N | 2-(2-amino-pyridin-3- |
| | phenyl | | phenylmethyl | | yl)ethylamino |
| 11 | 4-fluoro- | CH₂ | 4-methoxy- | N | 2-N-pyrrolidinyl- |
| | phenyl | | phenylmethyl | | pyridin-3-ylmethyl- |
| | | | | | amino |
| 12 | 4-methoxy- | CH₂ | 4-methoxy- | N | 2-n-piperazinyl- |
| | phenyl | | phenylmethyl | | pyridin-3-ylmethyl- |
| | | | | | amino |
| 13 | 4-methoxy- | CH₂ | 4-methoxy- | N | 2-N-piperidinyl- |
| | phenyl | | phenylmethyl | | pyridin-3-yl |
| | | | | | methyl-amino |
| 14 | 4-fluoro- | CH₂ | 4-methoxy- | N | 2-methylamino-pyridin- |
| | phenyl | | phenylmethyl | | 3-yl |
| | | | | | methyl-amino |
| 15 | 4-fluoro- | CH₂ | 4-methoxy- | N | 2-n-propylamino- |
| | phenyl | | phenylmethyl | | pyridin-3-yl |
| | | | | | methyl-amino |
| 16 | 4-fluoro- | CH₂ | 4-methoxy- | N | 2-n-butylamino- |
| | phenyl | | phenylmethyl | | pyridin-3-ylmethyl- |
| | | | | | amino |
| 17 | 4-fluoro- | CH₂ | 4-methoxy- | N | 2-N-morpholino- |
| | phenyl | | phenylmethyl | | pyridin-3-yl |
| | | | | | methyl-amino |
| 18 | 4-fluoro- | CH₂ | 4-methoxy- | N | 2-N-thiomorpholino- |
| | phenyl | | phenylmethyl | | pyridn-3-yl |
| | | | | | methyl-amino |
| 19 | 4-fluoro- | CH₂ | 4-methoxy- | N | 2-ethylamino-pyridin- |
| | phenyl | | phenylmethyl | | 3-yl |
| | | | | | methyl-amino |
| 20 | 4-methoxy- | CH₂ | 4-methoxy- | N | 2-N-morpholino- |
| | phenyl | | phenylmethyl | | pyridin-3-ylmethyl- |
| | | | | | amino |
| 21 | 4-fluoro- | CH₂ | 4-methoxy- | N | 1,2,3,4-tetrahydro-[1,8] |
| | phenyl | | phenylmethyl | | naphthyridin-7-yl |
| | | | | | methyl-amino |
| 22 | 4-methoxy- | CH₂ | 4-methoxy- | N | 4,6-dimethyl-pyridin-3- |
| | phenyl | | phenylmethyl | | ylmethyl-amino |
| 23 | benzofuran-2- | CH₂ | 4-methoxy- | N | 2-amino- |
| | yl | | phenylmethyl | | pyridin-3-yl |
| | | | | | methyl-amino |
| 24 | 4-methylthio- | CH₂ | 4-methoxy- | N | 2-amino- |
| | phenyl | | phenylmethyl | | pyridin-3-yl |
| | | | | | methyl-amino |
| 25 | 4-methoxy- | CH₂ | 4-methoxy- | N | 6-(4-fluoro-phenyl)- |
| | phenyl | | phenylmethyl | | pyridin-3-yl |
| | | | | | methyl-amino |
| 26 | 4-methoxy- | CH₂ | 4-methoxy- | N | 2-amino- |
| | phenyl | | phenylmethyl | | pyridin-3-yl |
| | | | | | methyl-amino |
| 27 | 4-fluoro- | CH₂ | 4-methoxy- | N | 2-(2-dimethylamino- |
| | phenyl | | phenylmethyl | | ethylamino)-pyridin-3- |
| | | | | | yl |
| | | | | | methyl-amino |
| 28 | 4-fluoro- | CH₂ | 4-methoxy- | N | 2-(2-methoxy- |
| | phenyl | | phenylmethyl | | ethylamino)-pyridin-3- |
| | | | | | yl |
| | | | | | methyl-amino |
| 29 | 4-fluoro- | CH₂ | 4-methoxy- | N | 2-(2-hydroxy- |
| | phenyl | | phenylmethyl | | ethylamino)-pyridin-3- |
| | | | | | yl |
| | | | | | methyl-amino |
| 30 | 4-fluoro- | CH₂ | 4-methoxy- | N | 2-(2-amino- methyl-amino |
| | phenyl | | phenylmethyl | | ethylamino)-pyridin-3- |
| | | | | | yl |
| 31 | 4-fluoro- | CH₂ | 4-methoxy- | N | 2-(2-cyclohexylamino- |
| | phenyl | | phenylmethyl | | pyridin-3-yl |
| | | | | | methyl-amino |
| 32 | 4-methoxy- | CH₂ | 4-methoxy- | N | N-oxo-2-amino- |
| | phenyl | | phenylmethyl | | pyridin-3-yl |
| | | | | | methylamino |
| 33 | 4-methoxy- | CH₂ | 4-hydroxy- | N | 2-amino- |
| | phenyl | | phenylmethyl | | pyridin-3-yl |
| | | | | | methyl-amino |
| 34 | 4-methoxy- | CH₂ | 4-methoxy- | N | 2-propylamino- |
| | phenyl | | phenylmethyl | | pyridin-3-yl |
| | | | | | methyl-amino |
| 35 | 4-methoxy- | CH₂ | 4- | N | 2-amino- |
| | phenyl | | difluoromethoxy | | pyridin-3-yl |
| | | | phenylmethyl | | methyl-amino |
| 36 | 4-methoxy- | CH₂ | 4- | N | 2-amino- |
| | phenyl | | methoxycarbonyl | | pyridin-3-yl |
| | | | phenylmethyl | | methyl-amino |
| 37 | 4-methoxy- | CH₂ | 4-methylcarbonyl | N | 2-amino- |
| | phenyl | | amino- | | pyridin-3-yl |
| | | | phenylmethyl | | methyl-amino |
| 38 | 4-methoxy- | CH₂ | 4- | N | 2-amino- |
| | phenyl | | trifluoromethoxy- phenylmethyl | | pyridin-3-yl |
| | | | | | methyl-amino |
| 39 | 4-methoxy- | CH₂ | 4-methoxy- | N | pyridin-2-yl |
| | phenyl | | phenylmethyl | | methyl-amino |
| 40 | 4-methoxy- | CH₂ | 4-methoxy- | N | pyridin-3-yl |
| | phenyl | | phenylmethyl | | methyl-amino |
| 41 | 4-methoxy- | CH₂ | 4-methoxy- | N | pyridin-4-yl |
| | phenyl | | phenylmethyl | | methyl-amino |
| 42 | 3-methoxy- | CH₂ | 4-methoxy- | N | 2-amino- |
| | phenyl | | phenylmethyl | | pyridin-3-yl |
| | | | | | methyl-amino |
| 43 | phenyl | CH₂ | 4-methoxy- | N | 2-amino- |
| | | | phenylmethyl | | pyridin-3-yl |
| | | | | | methyl-amino |
| 44 | 4-cyano- | CH₂ | 4-methoxy- | N | 2-amino- |
| | phenyl | | phenylmethyl | | pyridin-3-yl |
| | | | | | methyl-amino |
| 45 | 4-trifluoro | CH₂ | 4-methoxy- | N | 2-amino- |
| | methoxy- | | phenylmethyl | | pyridin-3-yl |
| | phenyl | | | | methyl-amino |
| 46 | 4-ethoxy- | CH₂ | 4-methoxy- | N | 2-amino- |
| | phenyl | | phenylmethyl | | pyridin-3-yl |
| | | | | | methyl-amino |
| 47 | 4-nitro-phenyl | CH₂ | 4-methoxy- | N | 2-amino- |
| | | | phenylmethyl | | pyridin-3-yl |
| | | | | | methyl-amino |
| 48 | 4-methoxy- | CH(allyl) | 4-methoxy- | N | 2-amino- |
| | phenyl | | phenylmethyl | | pyridin-3-yl |
| | | | | | methyl-amino |
| 49 | 4- phenyl | CH₂ | 4-methoxy- | N | 2-amino- |
| | trifluoromethyl- | | phenylmethyl | | pyridin-3-yl |
| | | | | | methyl-amino |
| 50 | 4-methoxy- | CH₂ | 4-methoxy- | N | 2-(2-methoxy- |
| | phenyl | | phenylmethyl | | ethylamino)-pyridin-3- |
| | | | | | yl |
| | | | | | methyl-amino |
| 51 | 4-methoxy- | CH₂ | 4-methoxy- | N | 2-(2-dimethylamino- |
| | phenyl | | phenylmethyl | | ethylamino)-pyridin-3- |
| | | | | | yl |
| | | | | | methyl-amino |
| 52 | 4-methoxy- | CH₂ | 4-aminocarbonyl- | N | 2-amino- |
| | phenyl | | phenylmethyl | | pyridin-3-yl |
| | | | | | methyl-amino |
| 53 | 4-methoxy- | CH₂ | 4-methoxy- | N | N-oxo- |
| | phenyl | | phenylmethyl | | pyridin-3-yl |
| | | | | | methyl-amino |
| 54 | 4-hydroxy- | CH₂ | 4-methoxy- | N | 2-amino- |
| | phenyl | | phenylmethyl | | pyridin-3-yl |
| | | | | | methyl-amino |
| 55 | 3-fluoro- | CH₂ | 4-methoxy- | N | 2-amino- |
| | phenyl | | phenylmethyl | | pyridin-3-yl |
| | | | | | methyl-amino |
| 56 | 4- | CH₂ | 4-methoxy- | N | 2-amino- |
| | methoxycarbonyl- | | phenylmethyl | | pyridin-3-yl |
| | phenyl | | | | methyl-amino |
| 57 | 4-methoxy- | CH₂ | 4-methoxy- | N | 2-amino-5-phenyl- |
| | phenyl | | phenylmethyl | | pyridin-3-yl |
| | | | | | methyl-amino |
| 58 | 4-methoxy- | CH₂ | 4-methoxy- | N | 2-amino-4-methoxy |
| | phenyl | | phenylmethyl | | pyridin-3-yl |
| | | | | | methyl-amino |
| 59 | 4-methoxy- | CH₂ | 4-methoxy- | N | 2-methyl- |
| | phenyl | | phenylmethyl | | pyridin-3-yl |
| | | | | | methyl-amino |
| 60 | 4-fluoro- | CH₂ | 4-methoxy- | N | 4,6-dimethyl-pyridin-3- |
| | phenyl | | phenylmethyl | | yl |
| | | | | | methyl-amino |
| 61 | 4-methoxy- | CH₂ | 4-methoxy- | CH | 4,6-dimethyl-pyridin-3- |
| | phenyl | | phenylmethyl | | yl |
| | | | | | methyl-amino |
| 62 | 4-methoxy- | CH₂ | 4-methoxy- | N | 4-methyl- |
| | phenyl | | phenylmethyl | | pyridin-2-yl |
| | | | | | methyl-amino |
| 63 | 4-methoxy- | CH₂ | 4-ethyl- | N | 2-amino- |
| | phenyl | | phenylmethyl | | pyridin-3-yl |
| | | | | | methyl-amino |
| 64 | 4-methoxy- | CH₂ | 4-methoxy- | N | 6-trifluoromethyl- |
| | phenyl | | phenylmethyl | | pyridin-2-yl |
| | | | | | methyl-amino |
| 65 | 4-methoxy- | CH₂ | 4-methoxy- | N | 3-methyl- |
| | phenyl | | phenylmethyl | | pyridin-3-yl |
| | | | | | methyl-amino |
| 66 | 4-methoxy- | CH₂ | 4-methoxy- | N | 2-(2-methylthio- |
| | phenyl | | phenylmethyl | | ethylamino)-pyridin-3-yl |
| | | | | | methyl-amino |
| 67 | 4-methoxy- | CH₂ | 4-methoxy- | N | 2-(3-methyl- |
| | phenyl | | phenylmethyl | | butylamino)-pyridin-3-yl |
| | | | | | methyl-amino |
| 68 | 4-methoxy- | CH₂ | 4-methoxy- | N | 2-(tetrahydro-furan-2-yl |
| | phenyl | | phenylmethyl | | methyl-amino)- |
| | | | | | pyridin-3-yl |
| | | | | | methyl-amino |
| 69 | 4-methoxy- | CH₂ | 4-methoxy- | N | 2-(furan-2-ylmethyl- |
| | phenyl | | phenylmethyl | | amino)-pyrdin-3-yl |
| | | | | | methyl-amino |
| 70 | 4-methoxy- | CH₂ | 4-methoxy- | N | 2-(N-ethyl-pyrrolidin-2- |
| | phenyl | | phenylmethyl | | ylmethyl-amino)- |
| | | | | | pyridin-3-yl |
| | | | | | methyl-amino |
| 71 | phenyl | CH₂ | 4-methoxy- | N | 2-(2-methoxy- |
| | | | phenylmethyl | | ethylamino)-pyridin-3- |
| | | | | | yl |
| | | | | | methyl-amino |
| 72 | phenoxy | CH₂ | 4-methoxy- | N | 2-(2-methoxy- |
| | | | phenylmethyl | | ethylamino)-pyridin-3- |
| | | | | | yl methyl-amino |
| 73 | 2,3-dihydro- | CH₂ | 4-methoxy- | N | 2-(2-methoxy- |
| | benzo[1,4]dioxin- | | phenylmethyl | | ethylamino)-pyridin-3- |
| | 2-yl | | | | yl |
| | | | | | methyl-amino |
| 74 | 4-nitro-phenyl | CH₂CH₂ | 4-methoxy- | N | 2-(2-methoxy- |
| | | | phenylmethyl | | ethylamino)-pyridin-3- |
| | | | | | yl |
| | | | | | methyl-amino |
| 75 | 4-methoxy- | CH₂ | 4-methythio- | N | 2-amino- |
| | phenyl | | phenylmethyl | | pyridin-3-yl |
| | | | | | methyl-amino |
| 76 | 4-methoxy- | CH₂ | pyridin-4-ylmethyl | N | 2-amino- |
| | phenyl | | | | pyridin-3-yl |
| | | | | | methyl-amino |
| 77 | 4-methoxy- | CH₂ | benzofuran-2- | N | 2-amino- |
| | phenyl | | ylmethyl | | pyridin-3-yl |
| | | | | | methyl-amino |
| 78 | 4-methoxy- | CH₂ | 5-methoxy-n- | N | 2-amino- |
| | phenyl | | pentyl | | pyridin-3-yl |
| | | | | | methyl-amino |
| 79 | 4-methoxy- | CH₂ | n-hexyl | N | 2-amino- |
| | phenyl | | | | pyridin-3-yl |
| | | | | | methyl-amino |
| 80 | 4-methoxy- | CH₂ | 3-methoxy- | N | 2-amino- |
| | phenyl | | phenylmethyl | | pyridin-3-yl |
| | | | | | methyl-amino |
| 81 | 4-methoxy- | CH₂ | 3-cyano- | N | 2-amino- |
| | phenyl | | phenylmethyl | | pyridin-3-yl |
| | | | | | methyl-amino |
| 82 | 4-methoxy- | CH₂ | 3-nitro- | N | 2-amino- |
| | phenyl | | phenylmethyl | | pyridin-3-yl |
| | | | | | methyl-amino |
| 83 | 4- | CH₂ | 4-methoxy- | N | 4,6-dimethyl-pyridin-3- |
| | difluoromethoxy- | | phenylmethyl | | yl |
| | phenyl | | | | methyl-amino |
| 84 | 4- | CH₂ | 4-methoxy- | N | 2-amino- |
| | difluoromethoxy- | | phenylmethyl | | pyridin-3-yl |
| | phenyl | | | | methyl-amino |
| 85 | 4- | CH₂ | 4- | N | 2-amino- |
| | difluoromethoxy- | | difluoromethoxy- | | pyridin-3-yl |
| | phenyl | | phenylmethyl | | methyl-amino |
| 86 | 4-methoxy- | CH₂ | 2-ethyl- | N | 2-amino- |
| | phenyl | | phenylmethyl | | pyridin-3-yl |
| | | | | | methyl-amino |
| 87 | 4-methoxy- | CH₂ | 2- | N | 2-amino- |
| | phenyl | | trifluoromethoxy- | | pyridin-3-yl |
| | | | phenylmethyl | | methyl-amino |
| 88 | 4-methoxy- | CH₂ | 2-cyano- | N | 2-amino- |
| | phenyl | | phenylmethyl | | pyridin-3-yl |
| | | | | | methyl-amino |
| 89 | 4-iodo-phenyl | CH₂ | 4-methoxy- | N | 2-amino- |
| | | | phenylmethyl | | pyridin-3-yl |
| | | | | | methyl-amino |
| 90 | 4-pyrazol-1-yl- | CH₂ | 4-methoxy- | N | 2-amino- |
| | phenyl | | phenylmethyl | | pyridin-3-yl |
| | | | | | methyl-amino |
| 91 | 4-fluoro- | CH₂ | 4- | N | 2-amino- |
| | phenyl | | trifluoromethoxy- | | pyridin-3-yl |
| | | | phenylmethyl | | methyl-amino |
| 92 | 4-methoxy- | CH₂ | 2-methoxy- | N | 2-amino- |
| | phenyl | | phenylmethyl | | pyridin-3-yl |
| | | | | | methyl-amino |
| 93 | 4-methoxy- | CH₂ | 3- | N | 2-amino- |
| | phenyl | | methoxycarbonyl- | | pyridin-3-yl |
| | | | phenylmethyl | | methyl-amino |
| 94 | 4-methoxy- | CH₂ | 2-(4-methoxy- | N | 2-amino- |
| | phenyl | | phenyl)-ethyl | | pyridin-3-yl |
| | | | | | methyl-amino |
| 95 | 4-methoxy- | CH₂ | 6-methoxy- | N | 2-amino- |
| | phenyl | | pyridin-3-ylmethyl | | pyridin-3-yl |
| | | | | | methyl-amino |
| 96 | 4-methoxy- | CH₂ | 4- | N | 4,6-dimethyl-pyridin-3- |
| | phenyl | | difluoromethoxy- | | ylmethyl-amino |
| | | | phenylmethyl | | |
| 97 | 4-methoxy- | CH₂ | 4-methoxy- | N | 2-amino-4,6-dimethyl- |
| | phenyl | | phenylmethyl | | pyridin-3-yl |
| | | | | | methyl-amino |
| 98 | 4-methoxy- | CH₂ | 3-trifluoromethoxy- | N | 2-amino- pyridin-3-yl |
| | phenyl | | phenylmethyl | | methyl-amino |
| 99 | 4-methoxy- | CH₂ | 3- | N | 4,6-dimethyl-pyridin-3- |
| | phenyl | | trifluoromethoxy- | | yl |
| | | | phenylmethyl | | methyl-amino |
| 100 | 4-methoxy- | CH₂ | 4-methylthio | N | 4,6-dimethyl-pyridin-3- |
| | phenyl | | phenylmethyl | | yl |
| | | | | | methyl-amino |
| 101 | 4-methoxy- | CH₂ | pyridin-4-ylmethyl | N | 4,6-dimethyl-pyridin-3- |
| | phenyl | | | | yl |
| | | | | | methyl-amino |
| 102 | 4-methoxy- | CH₂ | benzofuran-2- | N | 4,6-dimethyl- |
| | phenyl | | ylmethyl | | pyridin-3-ylmethyl- |
| | | | | | amino |
| 103 | 4-methoxy- | CH₂ | n-hexyl | N | 4,6-dimethyl- |
| | phenyl | | | | pyridin-3-yl |
| | | | | | methyl-amino |
| 104 | 4-methoxy- | CH₂ | 6-methoxy- | N | 4,6-dimethyl- |
| | phenyl | | pyridin-3-ylmethyl | | pyridin-3-yl |
| | | | | | methyl-amino |
| 105 | 4-methoxy- | CH₂ | 2- | N | 4,6-dimethyl- |
| | phenyl | | trifluoromethoxy- | | pyridin-3-yl |
| | | | phenylmethyl | | methyl-amino |
| 106 | 4-methoxy- | CH₂ | 2-methoxy- | N | 4,6-dimethyl-pyridin-3- |
| | phenyl | | phenylmethyl | | yl |
| | | | | | methyl-amino |
| 107 | 4-ethoxy- | CH₂ | 4-methoxy- | N | 4,6-dimethyl- |
| | phenyl | | phenylmethyl | | pyridin-3-yl |
| | | | | | methyl-amino |
| 108 | 4-nitro-phenyl | CH₂ | 4-methoxy- | N | 4,6-dimethyl- |
| | | | phenylmethyl | | pyridin-3-yl |
| | | | | | methyl-amino |
| 109 | 4-methoxy- | CH(allyl) | 4-methoxy- | N | 4,6-dimethyl- |
| | phenyl | | phenylmethyl | | pyridin-3-yl |
| | | | | | methyl-amino |
| 110 | 4-trifluoro- | CH₂ | 4-methoxy- | N | 4,6-dimethyl- |
| | phenyl | | phenylmethyl | | pyridin-3-yl |
| | | | | | methyl-amino |
| 111 | 3-methoxy- | CH₂ | 4-methoxy- | N | 4,6-dimethyl- |
| | phenyl | | phenylmethyl | | pyridin-3-yl |
| | | | | | methyl-amino |
| 112 | 3-fluoro- | CH₂ | 4-methoxy- | N | 4,6-dimethyl-pyridin-3- |
| | phenyl | | phenylmethyl | | yl |
| | | | | | methyl-amino |
| 113 | pyridin-4- | CH₂ | 4-methoxy- | N | 4,6-dimethyl- |
| | ylmethyl | | phenylmethyl | | pyridin-3-yl |
| | | | | | methyl-amino |
| 114 | 4- | CH₂ | 4-methoxy- | N | 4,6-dimethyl- |
| | methoxycarbonyl- | | phenylmethyl | | pyridin-3-yl |
| | phenyl | | | | methyl-amino |
| 115 | 4-methoxy- | CH₂ | 4-methoxy- | N | 6-amino- |
| | phenyl | | phenylmethyl | | pyridin-2-yl |
| | | | | | methyl-amino |
| 116 | 4-methoxy- | CH₂ | 4-fluoro- | N | 4,6-dimethyl- |
| | phenyl | | phenylmethyl | | pyridin-3-yl |
| | | | | | methyl-amino |
| 117 | 4-methoxy- | CH₂ | 4-chloro- | N | 4,6-dimethyl- |
| | phenyl | | phenylmethyl | | pyridin-3-yl |
| | | | | | methyl-amino |
| 118 | 4-methoxy- | CH₂ | 4-methoxy- | N | N-oxo-4,6-dimethyl- |
| | phenyl | | phenylmethyl | | pyridin-3-yl |
| | | | | | methyl-amino |
| 119 | indol-3-yl | CH₂ | 4-methoxy- | N | 2-amino- |
| | | | phenylmethyl | | pyridin-3-yl |
| | | | | | methyl-amino |
| 120 | 2,3-dihydro- | CH₂ | 4-methoxy- | N | 2-amino- |
| | benzo[1,4]dioxin- | | phenylmethyl | | pyridin-3-yl |
| | 2-yl | | | | methyl-amino |
| 121 | 4-methoxy- | CH₂ | 4-methoxy- | N | pyridin-3-ylmethoxy |
| | phenyl | | phenylmethyl | | |
| 122 | 4-methoxy- | CH₂ | 4-methoxy- | N | 6-trifluoromethyl- |
| | phenyl | | phenylmethyl | | pyridin-3-yl |
| | | | | | methyl-amino |
| 123 | 2,3-dihydro- | CH₂ | 4-methoxy- | N | 4,6-dimethyl- |
| | benzofuran-5- | | phenylmethyl | | pyridin-3-yl |
| | yl | | | | methyl-amino |
| 124 | 3-nitro-4- | CH₂ | 4-methoxy- | N | 2-amino- |
| | methoxy- | | phenylmethyl | | pyridin-3-yl |
| | phenyl | | | | methyl-amino |
| 125 | 4-methoxy- | CH₂ | 2,3-dihydro- | N | 2-amino- |
| | phenyl | | benzofuran-5-yl | | pyridin-3-yl |
| | | | methyl | | methyl-amino |
| 126 | 4-methoxy- | CH₂ | benzofuran-5-yl | N | 2-amino- |
| | phenyl | | methyl | | pyridin-3-yl |
| | | | | | methyl-amino |
| 127 | 4-methoxy- | CH₂ | indol-5-ylmethyl | N | 2-amino- |
| | phenyl | | | | pyridin-3-yl |
| | | | | | methyl-amino |
| 128 | 4-methoxy- | CH₂ | 2,3-dihydro- | N | 4,6-dimethyl- |
| | phenyl | | benzofuran-5-yl | | pyridin-3-yl |
| | | | methyl | | methyl-amino |
| 129 | 4-methoxy- | CH₂ | benzofuran-5-yl | N | 4,6-dimethyl- |
| | phenyl | | methyl | | pyridin-3-yl |
| | | | | | methyl-amino |
| 130 | 4-methoxy- | CH₂ | indol-5-ylmethyl | N | 4,6-dimethyl- |
| | phenyl | | | | pyridin-3-yl |
| | | | | | methyl-amino |
| 131 | 4-methoxy- | CH₂ | 4- | N | 2-amino- |
| | phenyl | | methanesulfonyl- | | pyridin-3-yl |
| | | | phenylmethyl | | methyl-amino |
| 132 | 4-methoxy- | CH₂ | 4- | N | 4,6-dimethyl- |
| | phenyl | | methanesulfonyl- | | pyridin-3-yl |
| | | | phenylmethyl | | methyl-amino |
| 133 | benzofuran-5- | CH₂ | 4-methoxy- | N | 4,6-dimethyl- |
| | yl | | phenylmethyl | | pyridin-3-yl |
| | | | | | methyl-amino |
| 134 | benzofuran-5- | CH₂ | 4-methoxy- | N | 2-amino- |
| | yl | | phenylmethyl | | pyridin-3-yl |
| | | | | | methyl-amino |
| 135 | 4-methoxy- | CH₂ | 4-t-butoxy- | N | 2-amino- |
| | phenyl | | phenylmethyl | | pyridin-3-yl |
| | | | | | methyl-amino |
| 136 | 4-methoxy- | CH₂ | 3-nitro-4-methoxy- | N | 4,6-dimethyl- |
| | phenyl | | phenylmethyl | | pyridin-3-yl |
| | | | | | methyl-amino |
| 137 | 4-methoxy- | CH₂ | 3-nitro-4-methoxy- | N | 2-amino- |
| | phenyl | | phenylmethyl | | pyridin-3-yl |
| | | | | | methyl-amino |
| 138 | 4-methoxy- | CH₂ | indol-4-ylmethyl | N | 2-amino- |
| | phenyl | | | | pyridin-3-yl |
| | | | | | methyl-amino |
| 139 | 4-methoxy- | CH₂ | indol-4-ylmethyl | N | 4,6-dimethyl- |
| | phenyl | | | | pyridin-3-yl |
| | | | | | methyl-amino |
| 140 | 4-methoxy- | CH₂ | benzothiophen-5- | N | 2-amino- |
| | phenyl | | ylmethyl | | pyridin-3-yl |
| | | | | | methyl-amino |
| 141 | 4-fluoro- | CH₂ | 4-methoxy- | N | 2-amino- |
| | phenyl | | phenylmethyl | | pyridin-3-yl |
| | | | | | methyl-amino |
| 142 | 4-methoxy- | CH₂ | benzothiophen-5- | N | 4,6-dimethyl- |
| | phenyl | | ylmethyl | | pyridin-3-yl |
| | | | | | methyl-amino |
| 143 | 2-methoxy- | CH₂ | 4-methoxy- | N | 2-amino- |
| | phenyl | | phenylmethyl | | pyridin-3-yl |
| | | | | | methyl-amino |
| 144 | 2-methoxy- | CH₂ | 4-methoxy- | N | 4,6-dimethyl- |
| | phenyl | | phenylmethyl | | pyridin-3-yl |
| | | | | | methyl-amino |
| 145 | benzothiphen- | CH₂ | 4-methoxy- | N | 2-amino- |
| | 5-yl | | phenylmethyl | | pyridin-3-yl |
| | | | | | methyl-amino |
| 146 | benzothiphen- | CH₂ | 4-methoxy- | N | 4,6-dimethyl- |
| | 5-yl | | phenylmethyl | | pyridin-3-yl |
| | | | | | methyl-amino |
| 147 | 4-methoxy- | CH₂ | 4-methoxy- | N | 6-n-propyl-amino- |
| | phenyl | | phenylmethyl | | pyridin-2-yl |
| | | | | | methyl-amino |
| 148 | 4-methoxy- | CH₂ | 4-methoxy- | N | 6-amino- |
| | phenyl | | phenylmethyl | | pyridin-2-yl |
| | | | | | methyl-amino |
| 149 | 4-methoxy- | CH₂ | 4-methoxy- | N | 2-amino- |
| | phenyl | | cyclohexylmethyl | | pyridin-3-yl |
| | | | | | methyl-amino |
| 150 | 4-methoxy- | CH₂ | 4-methoxy- | N | 4,6-dimethyl- |
| | phenyl | | cyclohexylmethyl | | pyridin-3-yl |
| | | | | | methyl-amino |
| 151 | 4-methoxy- | CH₂ | 3,4-dichloro- | N | 2-amino- |
| | phenyl | | phenylmethyl | | pyridin-3-yl |
| | | | | | methyl-amino |
| 152 | 4-methoxy- | CH₂ | 4-(isoindol-1,3- | N | 4,6-dimethyl- |
| | phenyl | | dione-2-yl)- | | pyridin-3-yl |
| | | | phenylmethyl | | methyl-amino |
| 153 | 4-methoxy- | CH₂ | 3-methoxy | N | 4,6-dimethyl- |
| | phenyl | | carbonyl-n-propyl | | pyridin-3-yl |
| | | | | | methyl-amino |
| 154 | 4-methoxy- | CH₂ | 4-methoxy- | N | 2-(pyridin-2-yl)- |
| | phenyl | | phenylmethyl | | ethylamino |
| 155 | 4-methoxy- | CH₂ | indol-4-ylmethyl | N | 2-amino-4,6-dimethyl- |
| | phenyl | | | | pyridin-3-yl |
| | | | | | methyl-amino |
| 156 | 4-fluoro- | CH₂ | 4- | N | 2-(pyridin-2-yl)- |
| | phenyl | | difluoromethoxy- | | ethylamino |
| | | | phenylmethyl | | |
| 157 | 4-methoxy- | CH₂ | 2,3-dihydro- | N | 2-amino-4,6-dimethyl- |
| | phenyl | | benzofuran-5-yl | | pyridin-3-yl |
| | | | methyl | | methyl-amino |
| 158 | 4-pyrazol-1-yl- | CH₂ | 4- | N | 4,6-dimethyl- |
| | phenyl | | difluoromethoxy- | | pyridin-3-yl |
| | | | phenylmethyl | | methyl-amino |
| 159 | 4-iodo-phenyl | CH₂ | 4- | N | 4,6-dimethyl- |
| | | | difluoromethoxy- | | pyridin-3-yl |
| | | | phenylmethyl | | methyl-amino |
| 160 | 4-fluoro- | CH₂ | 4- | N | 4,6-dimethyl- |
| | phenyl | | difluoromethoxy- | | pyridin-3-yl |
| | | | phenylmethyl | | methyl-amino |
| 161 | 4-methoxy- | CH₂ | 4- | N | 4,6-dimethyl- |
| | phenyl | | difluoromethoxy- | | pyridin-3-yl |
| | | | phenylmethyl | | methyl-amino |
| 162 | 4-trifluoromethyl- | CH₂ | 4- | N | 4,6-dimethyl- |
| | phenyl | | difluoromethoxy- | | pyridin-3-yl |
| | | | phenylmethyl | | methyl-amino |
| 163 | 4- | CH₂ | 4- | N | 4,6-dimethyl- |
| | difluoromethoxy- | | difluoromethoxy- | | pyridin-3-yl |
| | phenyl | | phenylmethyl | | methyl-amino |
| 164 | 4-cyano- | CH₂ | 4- | N | 4,6-dimethyl- |
| | phenyl | | difluoromethoxy- | | pyridin-3-yl |
| | | | phenylmethyl | | methyl-amino |
| 165 | 4- | CH₂ | 4- | N | 4,6-dimethyl- |
| | methoxycarbonyl- | | difluoromethoxy- | | pyridin-3-yl |
| | phenyl | | phenylmethyl | | methyl-amino |
| 166 | phenoxy | CH₂CH₂ | 4- | N | 4,6-dimethyl- |
| | | | difluoromethoxy- | | pyridin-3-yl |
| | | | phenylmethyl | | methyl-amino |
| 167 | 4-fluoro- | CH₂CH₂ | 4- | N | 4,6-dimethyl- |
| | phenoxy | | difluoromethoxy- | | pyridin-3-yl |
| | | | phenylmethyl | | methyl-amino |
| 168 | 4-[1,2,3] | CH₂ | 4- | N | 4,6-dimethyl- |
| | thiadiazol-4- | | difluoromethoxy- | | pyridin-3- |
| | yl- | | phenylmethyl | | ylmethyl- |
| | phenyl | | | | amino |
| 169 | 4-methoxy- | CH₂ | 4-methoxy- | CH | 2-(pyridin-2-yl)- |
| | phenyl | | phenylmethyl | | ethyl |
| 170 | 4-methoxy- | CH₂ | indol-6-ylmethyl | N | 2-amino- |
| | phenyl | | | | pyridin-3-yl |
| | | | | | methyl-amino |
| 171 | 4-methoxy- | CH₂ | indol-7-ylmethyl | N | 2-amino- |
| | phenyl | | | | pyridin-3-yl |
| | | | | | methyl-amino |
| 172 | 4-methoxy- | CH₂ | indol-7-ylmethyl | N | 4,6-dimethyl- |
| | phenyl | | | | pyridin-3-yl |
| | | | | | methyl-amino |
| 173 | 4-methylthio- | CH₂ | 4- | N | 2-amino-4,6-dimethyl- |
| | phenyl | | difluoromethoxy- | | pyridin-3-yl |
| | | | phenylmethyl | | methyl-amino |
| 174 | benzothiophen- | CH₂ | 4- | N | 2-amino-4,6-dimethyl- |
| | 5-yl | | difluoromethoxy- | | pyridin-3-yl |
| | | | phenylmethyl | | methyl-amino |
| 175 | benzofuran-5- | CH₂ | 4- | N | 2-amino-4,6-dimethyl- |
| | yl | | difluoromethoxy- | | pyridin-3-yl |
| | | | phenylmethyl | | methyl-amino |
| 176 | 2,3-dihydro- | CH₂ | 4- | N | 2-amino-4,6-dimethyl- |
| | benzofuran-5- | | difluoromethoxy- | | pyridin-3-yl |
| | yl | | phenylmethyl | | methyl-amino |
| 177 | 4-methylthio- | CH₂ | 4- | N | 4,6-dimethyl- |
| | phenyl | | difluoromethoxy- | | pyridin-3-yl |
| | | | phenylmethyl | | methyl-amino |
| 178 | benzofuran-5- | CH₂ | 4- | N | 4,6-dimethyl- |
| | yl | | difluoromethoxy- | | pyridin-3-yl |
| | | | phenylmethyl | | methyl-amino |
| 179 | 2,3-dihydro- | CH₂ | 4- | N | 4,6-dimethyl- |
| | benzofuran-5- | | difluoromethoxy- | | pyridin-3-yl |
| | yl | | phenylmethyl | | methyl-amino |
| 180 | 2-cyano- | CH₂ | 4- | N | 4,6-dimethyl- |
| | phenyl | | difluoromethoxy- | | pyridin-3-yl |
| | | | phenylmethyl | | methyl-amino |
| 181 | 4-hydroxy- | CH₂ | 4- | N | 4,6-dimethyl- |
| | phenyl | | difluoromethoxy- | | pyridin-3-yl |
| | | | phenylmethyl | | methyl-amino |
| 182 | 4-methylcarbonyloxy- | CH₂ | 4- | N | 4,6-dimethyl- |
| | phenyl | | difluoromethoxy- | | pyridin-3- |
| | | | phenylmethyl | | ylmethyl-amino |
| 183 | 4-methoxy- | CH₂ | 4-methoxy- | CH | 2-(pyridin-2-yl)- |
| | phenyl | | phenylmethyl | | ethyl |
| 184 | 4-methoxy- | CH₂ | 4-methoxy- | CH | cis-2-pyridin-4-yl- |
| | phenyl | | phenylmethyl | | vinyl |
| 185 | 2,3-dihydro- | CH₂ | 2,3-dihydro- | N | 2-amino-4,6-dimethyl- |
| | benzofuran-5- | | benzofuran-5-yl | | pyridin-3-yl |
| | yl | | methyl | | methyl-amino |
| 186 | benzofuran-5- | CH₂ | 2,3-dihydro- | N | 2-amino-4,6-dimethyl- |
| | yl | | benzofuran-5-yl | | pyridin-3-yl |
| | | | methyl | | methyl-amino |
| 187 | 4-methoxy- | CH₂ | 4-methoxy- | CH | 2-pyridin-2-yl- |
| | phenyl | | phenylmethyl | | ethyl |
| 188 | 4-methoxy- | CH₂ | 4-methoxy- | N | imidazo[1,2-α] |
| | | | | | pyridin-8-yl |
| | phenyl | | phenylmethyl | | methyl-amino |
| 189 | 4-methoxy- | CH₂ | 4-methoxy- | CH | 2-(2-aminocarbonyl- |
| | phenyl | | phenylmethyl | | pyridin-3-yl)-ethyl |
| 190 | 4-methoxy- | CH₂ | 4-methoxy- | CH | 2-amino- |
| | phenyl | | phenylmethyl | | pyridin-3-yl |
| | | | | | methoxy |
| 191 | 4- | CH₂ | 4- | N | 4,6-dimethyl- |
| | hydroxymethyl- | | difluoromethoxy- | | pyridin-3-yl |
| | phenyl | | phenylmethyl | | methyl-amino |
| 192 | 1-methyl-1H- | CH₂ | 4- | N | 4,6-dimethyl- |
| | benzotriazol-5- | | difluoromethoxy- | | pyridin-3-yl |
| | yl | | phenylmethyl | | methyl-amino |
| 193 | 2-methoxy- | CH₂ | 4- | N | 4,6-dimethyl- |
| | phenyl | | difluoromethoxy- | | pyridin-3-yl |
| | | | phenylmethyl | | methyl-amino |
| 194 | 4- | CH₂ | 4- | N | 4,6-dimethyl- |
| | aminocarbonyl- | | difluoromethoxy- | | pyridin-3-yl |
| | phenyl | | phenylmethyl | | methyl-amino |
| 195 | 2,6-difluoro-4- | CH₂ | 4- | N | 4,6-dimethyl- |
| | methoxy- | | difluoromethoxy- | | pyridin-3-yl |
| | phenyl | | phenylmethyl | | methyl-amino |
| 196 | benzo[1,2,3]thiadiazol- | CH₂ | 4- | N | 4,6-dimethyl- |
| | 5-yl | | difluoromethoxy- | | pyridin-3-yl |
| | | | phenylmethyl | | methyl-amino |
| 197 | methoxy | (CH₂)₅ | 4-methoxy- | N | 4,6-dimethyl- |
| | | | phenylmethyl | | pyridin-3-yl |
| | | | | | methyl-amino |
| 198 | methoxy | (CH₂)₅ | 4- | N | 4,6-dimethyl- |
| | | | difluoromethoxy- | | pyridin-3-yl |
| | | | phenylmethyl | | methyl-amino |
| 199 | 4-methoxy- | CH₂ | 4-methoxy- | CH | 2-(2-amino- |
| | phenyl | | phenylmethyl | | pyridin-3-yl)-ethyl |
| 200 | 4-methoxy- | CH₂ | 2,4-dimethoxy- | N | 2-amino- |
| | phenyl | | phenylmethyl | | pyridin-3-yl |
| | | | | | methyl-amino |
| 201 | 4-methoxy- | CH₂ | 4-methoxy- | N | 4-methyl- |
| | phenyl | | phenylmethyl | | pyridin-3-yl |
| | | | | | methyl-amino |
| 202 | 4-methoxy- | CH₂ | 4-methoxy- | CH | 2-amino-4,6-dimethyl- |
| | phenyl | | phenylmethyl | | pyridin-3-ylmethoxy |
| | | | | | methyl-amino |
| 203 | 4-methoxy- | CH₂ | 3-fluoro-4- | N | 2-amino-- |
| | phenyl | | methoxy- | | pyridin-3-yl |
| | | | phenylmethyl | | methyl-amino |
| 204 | 4-methoxy- | CH₂ | 3-fluoro-4- | N | 4,6-dimethyl- |
| | phenyl | | methoxy- | | pyridin-3-yl |
| | | | phenylmethyl | | methyl-amino |
| 205 | 4-methoxy- | CH₂ | 2-fluoro-4- | N | 2-amino- |
| | phenyl | | methoxy- | | pyridin-3-yl |
| | | | phenylmethyl | | methyl-amino |
| 206 | 4-methoxy- | CH₂ | 2-fluoro-4- | N | 4,6-dimethyl- |
| | phenyl | | methoxy- | | pyridin-3-yl |
| | | | phenylmethyl | | methyl-amino |
| 207 | benzo(1,3) | CH₂ | 4-methoxy- | N | 4,6-dimethyl- |
| | dioxal-5-yl | | phenylmethyl | | pyridin-3-yl |
| | | | | | methyl-amino |
| 208 | benzo(1,3) | CH₂ | 4- | N | 4,6-dimethyl- |
| | dioxal-5-yl | | difluoromethoxy- | | pyridin-3-yl |
| | | | phenylmethyl | | methyl-amino |
| 209 | 2,3-dihydro- | CH₂ | 4-methoxy- | N | 4,6-dimethyl- |
| | benzo[1,4] | | phenylmethyl | | pyridin-3-yl- |
| | dioxin-6-yl | | | | methylamino |
| 210 | 2,3-dihydro- | CH₂ | 4- | N | 4,6-dimethyl- |
| | benzo[1,4] | | difluoromethoxy- | | pyridin-3-yl |
| | dioxin-6-yl | | phenylmethyl | | methyl-amino |
| 211 | 4-methoxy- | CH₂ | 4-methoxy- | CH | pyridin-3- |
| | phenyl | | phenylmethyl | | ylmethylthio |
| 212 | 4-methoxy- | CH₂ | 2-methyl-2,3- | N | 2-amino-4,6-dimethyl- |
| | phenyl | | dihydro- | | pyridin-3-yl |
| | | | benzofuran-5-yl | | methyl-amino |
| | | | methyl | | |
| 213 | 4-methoxy- | CH₂ | 4-methoxy- | N | 2-(N-piperidinyl)-4,6- |
| | phenyl | | phenylmethyl | | dimethyl-pyridin-3-yl |
| | | | | | methyl-amino |
| 214 | 4-methoxy- | CH₂ | 4-methoxy- | CH | 2-(4-amino-pyridin- |
| | phenyl | | phenylmethyl | | 3-yl)-ethyl |
| 215 | 4-methoxy- | CH₂ | 4-methoxy- | N | 2-(pyridin-2-yl)- |
| | phenyl | | phenylmethyl | | ethylamino |
| 216 | 1-methyl-1H- | CH₂ | 4-methoxy- | N | 4,6-dimethyl- |
| | benzo | | phenylmethyl | | pyridin-3-yl |
| | triazol-5-yl | | | | methyl-amino |
| 217 | benzo[1,2,3]thiadiazol- | CH₂ | 4-methoxy- | N | 4,6-dimethyl- |
| | 5-yl | | phenylmethyl | | pyridin-3-yl |
| | | | | | methyl-amino |
| 218 | 3-fluoro-4- | CH₂ | 4-methoxy- | N | 4,6-dimethyl- |
| | methoxy- | | phenylmethyl | | pyridin-3-yl |
| | phenyl | | | | methyl-amino |
| 219 | benzo(1,3) | CH₂ | 4- | N | 2-amino-4,6-dimethyl- |
| | dioxal-5-yl | | difluoromethoxy- | | pyridin-3-yl |
| | | | phenylmethyl | | methyl-amino |
| 220 | benzo(1,3) | CH₂ | 4-methoxy- | N | 2-amino-4,6-dimethyl- |
| | dioxal-5-yl | | phenylmethyl | | pyridin-3-yl |
| | | | | | methyl-amino |
| 221 | 1-methyl-1H- | CH₂ | 4- | N | 2-amino-4,6-dimethyl- |
| | benzotriazol-5- | | difluoromethoxy- | | pyridin-3-yl |
| | yl | | phenylmethyl | | methyl-amino |
| 222 | 1-methyl-1H- | CH₂ | 4-methoxy- | N | 2-amino-4,6-dimethyl- |
| | benzotriazol-5- | | phenylmethyl | | pyridin-3-yl |
| | yl | | | | methyl-amino |
| 223 | 4-methoxy- | CH₂ | 4-methoxy- | CH | 2-(6-amino- |
| | phenyl | | phenylmethyl | | pyridin-2-yl)ethyl |
| 224 | 4-methoxy- | CH₂ | 5-methoxy-n- | N | 2-amino-4,6-dimethyl- |
| | phenyl | | pentyl | | pyridin-3-yl |
| | | | | | methyl-amino |
| 225 | 4-methoxy- | CH₂ | 4-methoxy- | CH | 1-(2-amino-pyridin-4- |
| | phenyl | | phenylmethyl | | yl)-ethoxy |
| 226 | 2,3-dihydro- | CH₂ | 2,3-dihydro- | N | N-oxo-2-amino-4,6- |
| | benzofuran-5- | | benzofuran-5-yl | | dimethyl-pyridin-3-yl |
| | yl | | methyl | | methyl-amino |
| 227 | indol-5-yl | CH₂ | 4- | N | 4,5-dimethyl- |
| | | | difluoromethoxy- | | pyridin-3-yl |
| | | | phenylmethyl | | methyl-amino |
| 228 | indol-5-yl | CH₂ | 4- | N | 2-amino- |
| | | | difluoromethoxy- | | pyridin-3-yl |
| | | | phenylmethyl | | methyl-amino |
| 229 | indol-5-yl | CH₂ | 4-methoxy- | N | 4,5-dimethyl- |
| | | | phenylmethyl | | pyridin-3-yl |
| | | | | | methyl-amino |
| 230 | indol-5-yl | CH₂ | 4-methoxy- | N | 2-amino- |
| | | | phenylmethyl | | pyridin-3-yl |
| | | | | | methyl-amino |
| 231 | 4-chloro- | CH₂ | 4-methoxy- | N | 2-amino- |
| | phenyl | | phenylmethyl | | pyridin-3-yl |
| | | | | | methyl-amino |
| 232 | 4-methoxy- | CH₂ | 4-methoxy- | CH | 2-amino- |
| | phenyl | | phenylmethyl | | pyrimidin-4-ylmethoxy |
| 233 | 2,3-dihydro- | CH₂ | 4- | N | N-oxo-2-amino-4,6- |
| | benzofuran-5- | | difluoromethoxy- | | dimethyl-pyridin-3-yl |
| | yl | | phenylmethyl | | methyl-amino |
| 234 | 4-methoxy- phenyl | CH₂ | 4-methoxy- phenylmethyl | N | 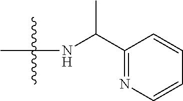 |
| 235 | 4-methoxy- phenyl | CH₂ | 4-methoxy- phenylmethyl | N | 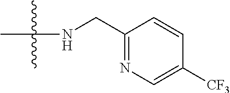 |
| 236 | 4-methoxy- phenyl | CH₂ | 4-methoxy- phenylmethyl | N | 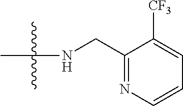 |
| 237 | 4-methoxy- phenyl | CH₂ | 4-methoxy- phenylmethyl | N | 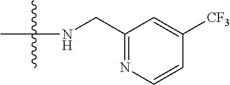 |
| 238 | 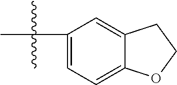 | CH₂ | —(CH₂)₅OCH₃ | N | 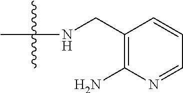 |
| 239 | 4-methoxy- phenyl | (CH₂)₂ | —(CH₂)₅OCH₃ | N | 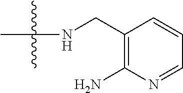 |
| 240 | 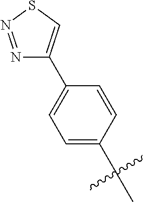 | CH₂ | 4-methoxy- phenylmethyl | N | 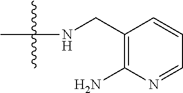 |
| 241 | 4-methoxy- phenyl | CH₂ | 4-methoxy- phenylmethyl | N | 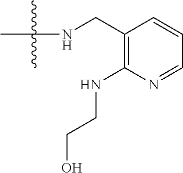 |
| 242 | 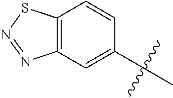 | CH₂ | 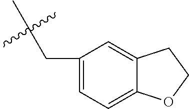 | N | 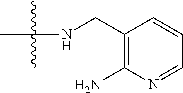 |
| 243 | 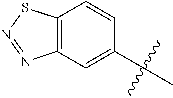 | CH₂ | 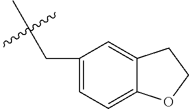 | N | 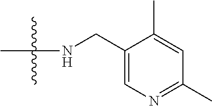 |
| 244 | 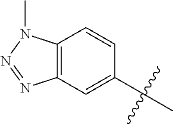 | CH₂ | 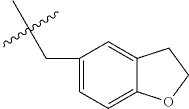 | N | 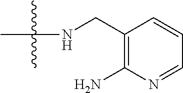 |
| 245 | 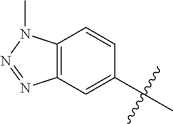 | CH₂ | 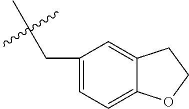 | N | 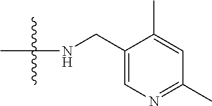 |
| 246 | 4-methoxy- phenyl | CH₂ | 4-methoxy- phenylmethyl | N | 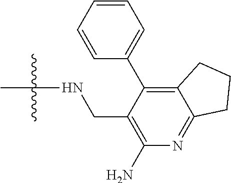 |
| 247 | 4-methoxy- phenyl | CH₂ | 4-methoxy- phenylmethyl | N | 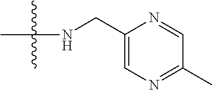 |
| 248 | 4-methoxy- phenyl | CH₂ | 4-methoxy- phenylmethyl | N | 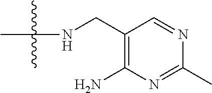 |
| 249 | 4-methoxy- phenyl | CH₂ | 4-methoxy- phenylmethyl | CH | 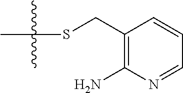 |
| 250 | 4-methoxy- phenyl | CH₂ | 4-methoxy- phenylmethyl | N | 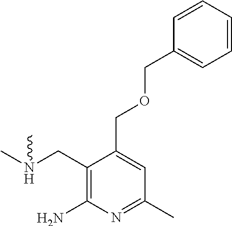 |
| 251 | 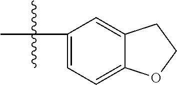 | CH₂ | 4- difluoromethoxy- phenylmethyl | N | 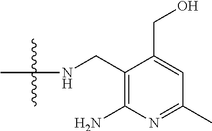 |
| 252 | methoxy | (CH₂)₅ | 4-methoxy- phenylmethyl | N | 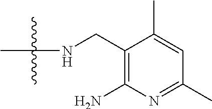 |
| 253 | 4-chloro- phenyl | CH₂ | —(CH₂)₅OCH₃ | N | 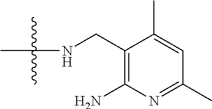 |
| 254 | phenyl | CH₂ | —(CH₂)₅OCH₃ | N | 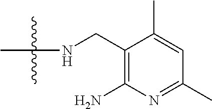 |
| 255 | 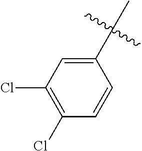 | CH₂ | —(CH₂)₅OCH₃ | N | 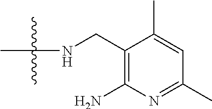 |
| 256 | 4-chloro- phenyl | CH₂ | —(CH₂)₅OCH₃ | N | 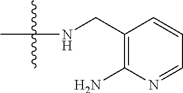 |
| 257 | 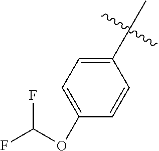 | CH₂ | —(CH₂)₅OCH₃ | N | 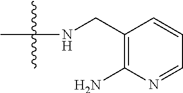 |
| 258 | 4-methoxy- phenyl | CH₂ | —(CH₂)₅OCH₃ | N | 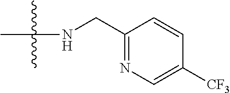 |
| 259 | 4-methoxy- phenyl | CH₂ | 4-methoxy- phenylmethyl | N | 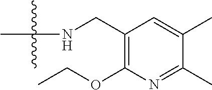 |
| 260 | 4-methoxy- phenyl | CH₂ | 4-methoxy- phenylmethyl | N | 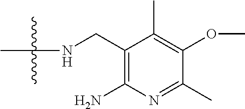 |
| 261 | 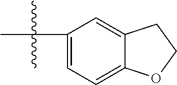 | CH₂ | 4- difluoromethoxy- phenylmethyl | N | 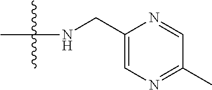 |
| 262 | 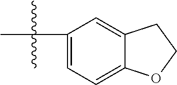 | CH₂ | 4- difluoromethoxy- phenylmethyl | N | 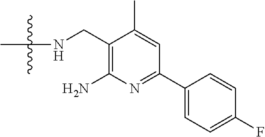 |
| 263 | 4-methoxy- phenyl | CH₂ | 4-methoxy- phenylmethyl | N | 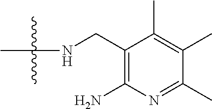 |
| 264 | 4-methoxy- phenyl | CH₂ | 4-methoxy- phenylmethyl | N | 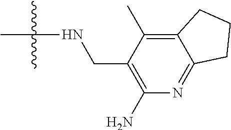 |
| 265 | 4-methoxy- phenyl | CH₂ | 4-methoxy- phenylmethyl | N | 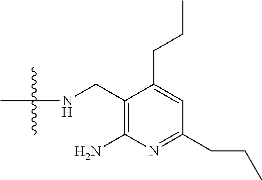 |
| 266 | CF₃ | (CH₂)₂ | 4-methoxy- phenylmethyl | N | 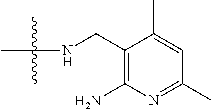 |
| 267 | 4-methoxy- phenyl | CH₂ | 4-methoxy- phenylmethyl | N | 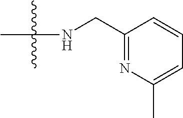 |
| 268 | 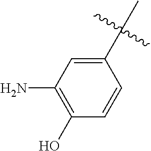 | CH₂ | 4- difluoromethoxy- phenylmethyl | N | 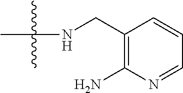 |
| 269 | 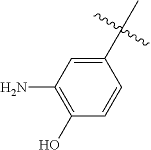 | CH₂ | 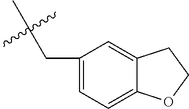 | N | 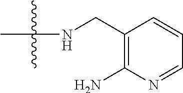 |
| 270 | 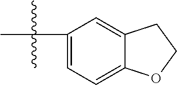 | CH₂ | 4- difluoromethoxy- phenylmethyl | N | 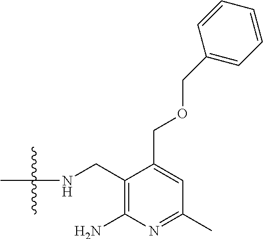 |
| 271 | 4-methoxy- phenyl | CH₂ | 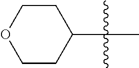 | N | 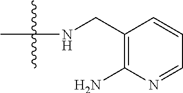 |
| 272 | 4-methoxy- phenyl | CH₂ | 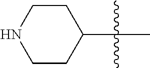 | N | 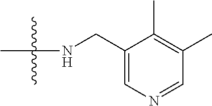 |

### BIOLOGICAL EXAMPLES

### Biological Example 1

### CFA-Induced Paw Radiant Heat Hypersensitivity

Each rat is placed in a test chamber on a warm glass surface and allowed to acclimate for approximately 10 min. A radiant thermal stimulus (beam of light) is then focused through the glass onto the plantar surface of each hind paw in turn. The thermal stimulus is automatically shut off by a photoelectric relay when the paw is moved or when the cut-off time is reached (20 sec for radiant heat at ˜5 amps). An initial (baseline) response latency to the thermal stimulus is recorded for each animal prior to the injection of complete Freund's adjuvant (CFA). Twenty-four hr following intraplantar CFA injection, the response latency of the animal to the thermal stimulus is then re-evaluated and compared to the animal's baseline response time. Only rats that exhibit at least a 25% reduction in response latency (i.e., were hyperalgesic) are included in further analysis. Immediately following the post-CFA latency assessment, the indicated test compound or vehicle is administered orally. Post-compound treatment withdrawal latencies are assessed at fixed time intervals, typically 30, 60, 120, 180, and 300 min.
The percent reversal (% R) of hypersensitivity is calculated using group mean values or using individual animal values, according to one of the following formulae:
1: For calculating the % R of hypersensitivity using the mean value for groups of animals at each time point:

% reversal=[(group treatment response−group CFA response)/(group baseline response−group CFA response)]×100
Results are given for the maximum % R observed at any time point tested.
2: For calculating the % R of hypersensitivity using individual animal values at each time point:

% reversal=[(individual treatment response−individual CFA response)/(individual baseline response−individual CFA response)]×100.
Results are given as a mean of the maximum % R values calculated for each individual animal ±SEM.

### Biological Example 2

### CFA-Induced Paw Pressure Hypersensitivity

Prior to testing, rats are aclimated to the handling procedure twice a day for a period of two days. The test consists of placing the left hindpaw on a polytetrafluoroethylene-coated platform and applying a linearly increasing mechanical force (constant rate of 12.5 mmHg/s) in between the third and fourth metatarsal of the dorsum of the rat's hindpaw, with a dome-tipped plinth (0.7 mm in radius), using an analgesy-meter (Stoelting, Chicago, Ill.), also known as a Randall-Selitto apparatus. The endpoint is automatically reached upon hindpaw withdrawal, and the terminal force (in grams) is noted. An initial (baseline) response threshold to the mechanical stimulus is recorded for each animal prior to the injection of complete Freund's adjuvant (CFA). Forty hr following intraplantar CFA injection, the response threshold of the animal to the mechanical stimulus is re-evaluated and compared to the animal's baseline response threshold. A response is defined as a withdrawal of the hindpaw, a struggling to remove the hindpaw or vocalization. Only rats that exhibit at least a 25% reduction in response threshold (i.e., hyperalgesia) are included in further analysis. Immediately following the post-CFA threshold assessment, rats are administered the indicated test compound or vehicle. Post-treatment withdrawal thresholds are assessed at 1 hr. Paw withdrawal thresholds are converted to percent reversal of hypersensitivity according to the following formula: % reversal=[(post treatment response−predose response)/(baseline response−predose response)]×100.

### Biological Example 3

### Visceral Hyperalgesia Model

This protocol uses barostat-controlled, isobaric colorectal distensions (CRD) in rats to evaluate the potency and efficacy of test compounds in treating visceral hyperalgesia. Rats (male Sprague-Dawley (275-350 g; Charles River Labs) are housed 2 to 4 animals per cage in a temperature and humidity controlled room with a 12 hr/12 hr light/dark cycle, with ad libitum access to food and water. One day after release from quarantine, the animals are acclimated to progressively longer (30 min and 4 hr later, 45 min) periods of simple restraint in plexiglas devices (G-3, rat ECU; Braintree Scientific; Braintree Mass.). The animals are returned to their home cages overnight. The next day they are acclimated in the restraint device for 60 min in the morning. Four hr later, the animals are lightly anesthetized with 70% CO₂:30% O₂. A highly compliant, 4 cm long polyethylene balloon, lubricated with lubricating jelly, is then inserted via the anus into the rectum and distal colon. The balloon is positioned such that the aboral end is 1 cm from the anus and is secured in place by taping the balloon catheter to the base of the tail. The catheter is connected to a computerized barostat that controls the inflation of the balloon and the resulting colorectal distension. The balloon pressure, representing intracolonic pressure, is continuously recorded. CRD in conscious animals elicits a reflex visceromotor response consisting of contraction of the anterior abdominal wall muscles (Ness T J and Gebhart G F; Colorectal distension as a noxious visceral stimulus: physiologic and pharmacologic characterization of pseudaffective reflexes in the rat, Brain Res., (1988), 450: 153-169). Contraction of these muscles increases intraabdominal pressure and subsequently increases intracolonic pressure. Changes in intracolonic pressure are transduced through the same balloon used to deliver the CRD. The manometric endpoint has recently been reported to mimic electromyographic responses recorded from anterior abdominal wall muscles in rats (Tammpere A, Brusberg M, Axenborg J, Hirsch I, Larsson H and Lindstrom E, Evaluation of pseudo-affective responses to noxious colorectal distension in rats by manometric recordings, Pain, (2005), 116: 220-226) Stimulus-response data are obtained by delivering two series of 20-sec ramp (15, 30, 45, 60, 75 mmHg) distensions at four-min intervals and recording the manometric response as follows: the intracolonic pressure signal is passed through a digital 1 Hz highpass filter, rectified and the integral of the initial 15 seconds of the CRD subjected to baseline subtraction (the 15 sec immediately preceding balloon distension); the responses at each distending pressure are averaged to obtain a control stimulus/response curve for each animal. The colorectal balloons are then removed and the animals are returned to their home cages.

The following morning, one treatment group is injected i.p. with test article or vehicle. One hour later, an acute colitis is induced in all treatment groups by the intracolonic instillation of a 1.5 mL bolus of 2.5% (w/v) zymosan A (from Saccharomyces cerevisiae; Sigma Chemical Co., St. Louis) in 30% ethanol (under light 70% CO₂:30% O₂ anesthesia). Four hours later, the animals are lightly anesthetized and the colorectal balloons inserted as on the previous day for controlled distensions. The identical CRD stimuli is applied and manometric responses are recorded and analyzed as described for the control phase of the experiment. Data are excluded from experiments in which animals in the vehicle treatment group do not exhibit a hyperalgesic response following zymosan administration. Data are expressed as a percent (%±SEM) of the initial (control) manometric responses, with each animal serving as its own control.

### Biological Example 4

### Models of Nociception

### Rat Formalin Test

Rats are administered vehicle or a test antinociceptive agent Animals are then placed in observation chambers and allowed to acclimate. Formalin (50 μL of 5%) is injected beneath the skin on the top of one hindpaw. The resulting biphasic pattern of activity, consisting of lifting, licking, biting and/or guarding (Wheeler-Aceto and Cowan, 1991) is quantified with an Automated Flinch Detecting System for 60 minutes. (Yaksh et al., 2001). Responses may be grouped by time into Phase I (1-9 min.), Phase II (10-60 min.) and/or Phase IIA (10-40 min.). Data are calculated as the percent maximum possible effect:

% MPE=100×(Mean Animal Drug Treated Count)/(Mean Animal vehicle Treated Count)

### REFERENCES

Wheeler-Aceto H and Cowan A. Standardization of the rat paw formalin test for the evaluation of analgesics. Psychopharmacol. 1991; 104:35-44.
Yaksh T L, Ozaki G, McCumber D, Rathbun M, Svensson C, Malkmus S, and Yaksh M C. An automated flinch detecting system for use in the formalin nociceptive bioassay. J Applied Physiology. 2001; 90:2386-402.

### Biological Example 5

### Antinociceptive Tests

### Mouse Acetylcholine-Induced Abdominal Irritant Test

The procedure used is that described by Collier et al. (1968), with minor modifications. Thirty minutes after the administration of test drug, the animals receive an i.p. injection of 5.5 mg/kg of acetylcholine bromide. The mice are then placed into large glass animal jars and continuously observed for the first occurrence of a characteristic behavioral response (i.e., twisting and elongation of the body, which extends throughout the hindlimbs) within the specified observation period of 10 minutes. The percent of inhibition of this response is calculated as follows:

% Inhibition=100×(Number of Nonresponders)/Number of Animals in Group)

The estimated ED₅₀ value (the dose of agonist calculated to produce 50% antinociception) and the corresponding 95% fiducial intervals are determined using the probit analysis of Litchfield and Wilcoxon (1949).

### REFERENCES

Litchfield JT and Wilcoxon F. A simplified method of evaluating dose-effect experiments. J Pharmacol Exp Ther 95: 1098-1104, 1949.

### Biological Example 6

### Antinociceptive Tests

### Mouse 48° C. Hot-Plate Test

The procedure used is that described by Eddy and Leimbach (1953) and O'Callaghan and Holtzman (1975), with minor modifications. Mice are placed on a heated surface (48° C.), and the time interval (seconds) between placement and the prototypic behavior (i.e., a shaking, licking or tucking of the hind paw) is recorded as the predrug latency response. This same procedure is repeated at 30 minutes after test drug is administered p.o., 10 mL/kg. The percent maximum possible antinociceptive effect (% MPE) is determined using the formula:

% MPE=100×(Test latency−Predrug latency)/(Cutoff time−Predrug Latency)

using the predrug latency of each animal and cut-off time established to prevent injury to the animal (i.e., 90 seconds). The ED₅₀ value and 95% confidence intervals are determined using a computer-assisted linear regression analysis of the dose-response curve, including an analysis of variance test for linearity.

### REFERENCES

Collier H O, Dinneen L C, Johnson C A and Schneider, C. The abdominal irritant response and its suppression by analgesic drugs in the mouse. Br J Pharmacol 32:295-310, 1968.
Eddy N B, Leimbach D. Synthetic analgesics II. Dithienylbutenyl- and dithienylbutylamines J Pharmacol Exp Ther 1953; 107:385-393.
O'Callaghan J P, Holtzman S G. Quantification of the analgesic activity of narcotic antagonists by a modified hot-plate procedure. J Pharmacol Exp Ther 1975; 192:497-505.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating or preventing pain comprising administering to a subject in need thereof, a therapeutically effective amount of a compound of Formula (I) 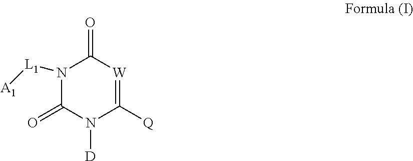
or an enantiomer, diastereomer, solvate, or pharmaceutically acceptable salt thereof;
wherein:
A₁ is CF₃, C₁₋₄alkoxy, aryl, aryloxy, benzofused heterocyclyl, or heteroaryl; wherein aryl, aryloxy, and heteroaryl are optionally substituted with pyrazol-1-yl or [1,2,3]thiadiazol-4-yl; or aryl, aryloxy, the benzo portion of benzofused heterocyclyl, and heteroaryl are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl; provided that A₁ is other than 3,5-di-t-butyl-phenyl;
L₁ is —(CH₂)ᵣ—, —CH₂C₂₋₄alkenyl-, or —CH₂CH₂X(CH₂)ₛ—, wherein L₁ is optionally substituted with one to two substituents independently selected from the group consisting of C₁₋₆alkyl, C₂₋₆alkenyl, C₂₋₆alkynyl, and halogen; and, r is an integer of 1 to 5; such that r is greater than or equal to 4 when A₁ is C₁₋₄alkoxy;
s is an integer of 1 to 3;
X is O or S;
D is —P-A₂;
wherein P is —(CH₂)₁₋₂— or —CH₂CH═CH— when A₂ is phenyl, benzofused heterocyclyl, heteroaryl, or C₃₋₈cycloalkyl; alternatively, P is —(CH₂)₃₋₆—, when A₂ is hydrogen, C₁₋₄alkoxy, or C₁₋₄alkoxycarbonyl; and wherein P is optionally substituted with one to two substituents independently selected from the group consisting of C₁₋₆alkyl, C₂₋₆alkenyl, C₂₋₆alkynyl, and halogen;
A₂ is hydrogen, C₁₋₄alkoxy, C₁₋₄alkoxycarbonyl, phenyl, benzofused heterocyclyl, heteroaryl, tetrahydro-pyranyl, piperidinyl, or C₃₋₈cycloalkyl; wherein phenyl, heteroaryl, the benzo portion of benzofused heterocyclyl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, N-isoindole-1,3-dione, C₁₋₆alkylthio, C₁₋₆alkylsulfonyl, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; such that no more than two substituents on A₂ are aryl(C₁₋₆)alkoxy, phenyl, N-isoindole-1,3-dione, or a non fused C₃₋₆cycloalkyloxy;
provided that A₂ is other than 3,5-di-t-butyl-phenyl;
W is N or C(R_{W}); wherein R_{W} is H or C₁₋₂alkyl;
Q is selected from the group consisting of (a) to (g), wherein
(a) is —NH(CH₂)₂—Ar₁ wherein Ar₁ is pyridinyl optionally substituted with one to three C₁₋₄alkyl substituents or a substituent selected from the group consisting of C₁₋₄alkoxy and amino;
provided that when Ar₁ is an unsubstituted pyridin-3-yl or unsubstituted pyridin-4-yl, and A₂ is 4-methoxy-phenyl, A₁ is other than unsubstituted phenyl or 3,4-dichloro-phenyl;
(b) is —NHCH(R_{z})-Ar₂ wherein R_{z} is H or C₁₋₃alkyl; Ar₂ is pyridinyl, pyrimidinyl, pyrazinyl, 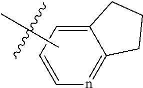
1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; and wherein Ar₂ is optionally substituted with one to three substituents independently selected from the group consisting of C₁₋₄alkyl, trifluoromethyl, hydroxyl-C₁₋₄alkyl, amino(C₁₋₄alkyl, (C₁₋₄alkyl)amino-(C₁₋₄)alkyl, di(C₁₋₄alkyl)amino-(C₁₋₄alkyl, C₁₋₄alkoxy, C₃₋₈cycloalkylamino, amino, (C₁₋₆alkyl)amino, and di(C₁₋₆alkyl)amino; or Ar₂ is optionally substituted with one amino group and three substituents independently selected from the group consisting of C₁₋₄alkyl and C₁₋₄alkoxy;
wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, C₁₋₄alkylthio, hydroxy, a 5 to 6 membered heteroaryl, or a 5 to 6 membered heterocyclyl; wherein a nitrogen atom of the 5 to 6 membered heterocyclyl is optionally substituted with a C₁₋₄alkyl substituent;
and wherein pyridin-2-yl and pyridin-3-yl are optionally further substituted with N-pyrrolidinyl, N-piperazinyl, N-piperidinyl, N-morpholinyl, N-thiomorpholinyl, —CH₂—O—CH₂—PH, and phenyl; wherein the phenyl substituent of pyridin-2-yl and pyridin-3-yl is optionally substituted with one to three substituents independently selected from the group consisting of C₁₋₄alkyl, C₁₋₄alkoxy, and halogen;
provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;
provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;
provided that when Q is —NHCH₂(2-amino-pyridin-3-yl), and A₁ is benzotriazol-1-yl, A₂ is other than 4-difluoromethoxy-phenyl;
provided that when Q is —NHCH₂(2-amino-pyridin-3-yl), L₁ is —(CH₂)₃—, and A₁ is pyrrol-1-yl, A₂ is other than 4-methoxy-phenyl;
provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
provided that when Q is —NHCH₂(2-amino-pyridin-3-yl), and A₁ is 4-fluoro-phenyl, A₂ is other than 4-fluoro-phenyl;
provided that when Q is —NHCH₂(6-amino-pyridin-2-yl), and A₁ is 4-fluoro-phenyl, A₂ is other than 4-trifluoromethoxy-phenyl;
provided that when Q is —NHCH₂(6-methyl-pyridin-2-yl), and A₁ is 4-methoxy-phenyl, A₂ is other than 4-methoxy-phenyl;
provided that when Q is —NHCH₂(imidazo[1,2-a]pyridinyl), and A₁ is 4-fluoro-phenyl, A₂ is other than 4-methoxy-phenyl;
provided that when Q is —NHCH₂(pyridin-4-yl), and A₁ is unsubstituted phenyl or 3,4-dichloro-phenyl, A₂ is other than 4-methoxy-phenyl;
provided that when Q is —NHCH₂(4,6-dimethyl-pyridin-3-yl), and A₁ is 4-methoxy-phenyl, —P-A₂ is other than —(CH₂)₅-methoxy;
provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
(c) is —CH₂NHCH₂—Ar₃, wherein W is N or CH, and Ar₃ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and that the point of attachment to quinolinyl is at the 2, 3, or 4-position; wherein Ar₃ is optionally substituted with one to three substituents independently selected from the group consisting of C₁₋₄alkyl, amino(C₁₋₄alkyl, (C₁₋₄alkyl)amino-(C₁₋₄alkyl, di(C₁₋₄alkyl)amino-(C₁₋₄alkyl, C₁₋₄alkoxy, amino, (C₁₋₆alkyl)amino, and di(C₁₋₆alkyl)amino;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(d) is —(CH₂)₂—Ar₄, wherein Ar₄ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₄ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(e) is —CH═CH—Ar₅; wherein Ar₅ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₅ is optionally substituted with one to three substituents independently selected from the group consisting of C₁₋₄alkyl, amino(C₁₋₄alkyl, di(C₁₋₄alkyl)amino-(C₁₋₄)alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(f) is —O—CH(R₁)—Ar₆ when W is CH; or, (f) is —S—CH(R₁)—Ar₆ and W is N or CH; wherein R₁ is hydrogen or C₁₋₄alkyl, and Ar₆ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;
wherein Ar₆ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
provided that when Q is —O—CH(R₁)—Ar₆, A₁ and A₂ are 4-methoxy-phenyl, and R₁ is hydrogen, Ar₆ is other than unsubstituted pyridin-2-yl or 2-amino-pyridin-4-yl;
and
(g) is —X₁—(CH(Rₓ))-2—Ar₇ when W is CH; wherein X₁ is O or S, Rₓ is H or C₁₋₄alkyl, and Ar₇ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;
wherein Ar₇ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
provided that when Q is —O (CH₂)₂—Ar₇ and A₁ and A₂ are 4-methoxy-phenyl, Ar₇ is other than unsubstituted pyridin-2-yl or unsubstituted pyridin-3-yl;
wherein a nitrogen atom of Ar₁, Ar₂, Ar₃, Ar₄, Ar₅, Ar₆, and Ar₇ is optionally substituted with oxo.

2. A method as in claim 1 wherein the pain is inflammatory pain.

3. A method as in claim 1 wherein the pain is visceral pain.

4. A method as in claim 1 wherein the pain is acute pain.

5. The use of a compound as in claim 1 for the preparation of a medicament or pharmaceutical composition for the treatment of inflammatory pain, visceral pain, or acute pain, in a subject in need thereof.

6. A method of treating or preventing pain comprising administering to a subject in need thereof, a therapeutically effective amount of a compound of Formula (I) 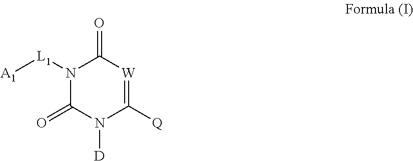
or an enantiomer, diastereomer, solvate, or pharmaceutically acceptable salt thereof;
wherein:
A₁ is substituted phenyl, benzotriazolyl, benzofuranyl, benzo[1,3]dioxalyl or 2,3-dihydro-benzofuranyl; wherein phenyl is substituted at the 4-position with methoxy, fluoro, or methylthio; and wherein A₁ other than substituted phenyl is optionally substituted with one to two substituents independently selected from the group consisting of methyl, methoxy, fluoro and methylthio;
L₁ is —CH₂—;
D is —P-A₂;
wherein P is —CH₂— when A₂ is phenyl, 2,3-dihydro-benzofuranyl, indolyl, benzofuranyl, pyridin-3-yl, or benzothiophenyl; alternatively, P is —(CH₂)₄₋₆—, when A₂ is C₁₋₄alkoxy;
A₂ is C₁₋₄alkoxy, phenyl, 2,3-dihydro-benzofuranyl, indolyl, benzofuranyl, pyridin-3-yl, or benzothiophenyl; wherein A₂ other than C₁₋₄alkoxy is optionally substituted with one to two substituents independently selected from the group consisting of C₁₋₄alkoxy, fluoro, fluorinated C₁₋₄-alkoxy, C₁₋₄alkylthio, C₁₋₄alkylsulfonyl, C₁₋₄alkoxycarbonyl, nitro, and hydroxy;
W is N;
Q is —NHCH₂—Ar₂ wherein Ar₂ is unsubstituted pyridin-2-yl, 4,6-dimethyl-pyridin-3-yl, 2-amino-pyridin-3-yl, or 2-((C₁₋₄alkyl)amino)-pyridin-3-yl;
wherein the C₁₋₄alkyl group of (C₁₋₄alkyl)amino is optionally substituted with di(C₁₋₄alkyl)amino, C₁₋₄alkoxy, or hydroxy;
and wherein 2-amino-pyridin-3-yl is optionally further substituted with 4,6-dimethyl or 4-methoxy;
provided that when Q is —NHCH₂(2-amino-pyridin-3-yl), and A₁ is pyridin-4-yl or 4-methyl-phenyl, A₂ is other than 4-methoxy-phenyl;
provided that when Q is —NHCH₂(2-amino-pyridin-3-yl), and A₁ is benzotriazol-1-yl, A₂ is other than 4-difluoromethoxy-phenyl;
provided that when Q is —NHCH₂(2-amino-pyridin-3-yl), and A₁ is 4-fluoro-phenyl, A₂ is other than 4-fluoro-phenyl;
provided that when Q is —NHCH₂(6-amino-pyridin-2-yl), and A₁ is 4-fluoro-phenyl, A₂ is other than 4-trifluoromethoxy-phenyl;
provided that when Q is —NHCH₂(6-methyl-pyridin-2-yl), and A₁ is 4-methoxy-phenyl, A₂ is other than 4-methoxy-phenyl;
provided that when Q is —NHCH₂(imidazo[1,2-a]pyridinyl), and A₁ is 4-fluoro-phenyl, A₂ is other than 4-methoxy-phenyl;
provided that when Q is —NHCH₂(4,6-dimethyl-pyridin-3-yl), and A₁ is 4-methoxy-phenyl, —P-A₂ is other than —(CH₂)₅-methoxy;
provided that when Q is —NHCH₂(4,6-dimethyl-pyridin-3-yl) and A₁ is 4-methoxy-phenyl, A₂ is other than 3-methoxy-phenyl or 3-nitro-phenyl; and
provided that when Q is —NHCH₂(4,6-dimethyl-pyridin-3-yl) and A₁ is benzotriazol-1-yl, A₂ is other than 4-difluoromethoxy-phenyl;
wherein a nitrogen atom of Ar₂ and Ar₄ is optionally substituted with oxo.

7. A method as in claim 6 wherein the pain is inflammatory pain.

8. A method as in claim 6 wherein the pain is visceral pain.

9. A method as in claim 6 wherein the pain is acute pain.

10. The use of a compound as in claim 6 for the preparation of a medicament or pharmaceutical composition for the treatment of inflammatory pain, visceral pain, or acute pain, in a subject in need thereof.

11. A method of treating or preventing pain comprising administering to a subject in need thereof, a therapeutically effective amount of a compound of Formula (I) 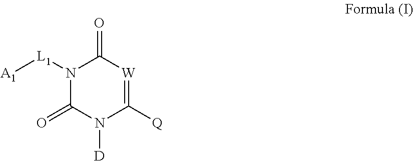
or pharmaceutically acceptable salt thereof;
selected from the group consisting of
a compound of Formula (I) wherein A₁ is 4-methoxy-phenyl, L₁ is CH₂, D is 4-methoxy-phenylmethyl, W is N, and Q is 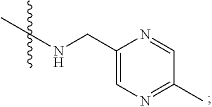
a compound of Formula (I) wherein A₁ is 4-methoxy-phenyl, L₁ is CH₂, D is 4-methoxy-phenylmethyl, W is N, and Q is 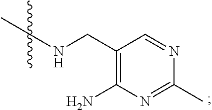
a compound of Formula (I) wherein A₁ is 4-chloro-phenyl, L₁ is CH₂, D is —(CH₂)₅OCH₃, W is N, and Q is 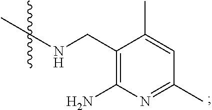
a compound of Formula (I) wherein A₁ is 3,4-dichloro-phenyl, L₁ is CH₂, D is 4-methoxy-phenylmethyl, W is N, and Q is 2-(pyridin-2-yl)ethyl-amino;
a compound of Formula (I) wherein A₁ is 3,4-dichloro-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 5-amino-pyridin-2-ylmethyl-amino;
a compound of Formula (I) wherein A₁ is 4-chloro-phenyl, L₁ is CH₂, D is 4-methoxy-phenylmethyl, W is N, and Q is 6-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-amino-pyrimidin-5-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-aminomethyl;
a compound of Formula (I) wherein A₁ is 4-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-quinolin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-pyridin-3-yl)-ethylamino;
a compound of Formula (I) wherein A₁ is 4-fluoro-phenyl, L₁ is CH₂, D is 4-methoxy-phenylmethyl, W is N, and Q is 2-N-pyrrolidinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperazinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperidinyl-pyridin-3-ylmethyl-amino;
a compound of Formula (I) wherein A₁ is 4-fluoro-phenyl, L₁ is CH₂, D is 4-methoxy-phenylmethyl, W is N, and Q is 2-methylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-butylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-thiomorpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-ethylamino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 1,2,3,4-tetrahydro-[1,8]naphthyridin-7-ylmethyl-amino;
a compound of Formula (I) wherein A₁ is 4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-2-yl, L₁ is CH₂, D is 4-methoxy-phenylmethyl, W is N, and Q is 2-amino-pyridin-3-ylmethyl-amino;
a compound of Formula (I) wherein A₁ is 4-methylthio-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-(4-fluoro-phenyl)-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-hydroxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-cyclohexylamino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-2-amino-pyridin-3-ylmethyl-amino;
a compound of Formula (I) wherein A₁ is 4-methoxy-phenyl, L₁ is CH₂, D is 4-hydroxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methylcarbonylamino-phenylmethyl, W is N, and Q is 2-amino-pyridin-3-ylmethyl-amino;
a compound of Formula (I) wherein A₁ is 4-methoxy-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-2-ylmethyl-amino;
a compound of Formula (I) wherein A₁ is 4-methoxy-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-4-ylmethyl-amino;
a compound of Formula (I) wherein A₁ is 3-methoxy-phenyl, L₁ is CH₂, D is 4-methoxy-phenylmethyl, W is N, and Q is 2-amino-pyridin-3-ylmethyl-amino;
a compound of Formula (I) wherein A₁ is phenyl, L₁ is CH₂, D is 4-methoxy-phenylmethyl, W is N, and Q is 2-amino-pyridin-3-ylmethyl-amino;
a compound of Formula (I) wherein A₁ is 4-cyano-phenyl, L₁ is CH₂, D is 4-methoxy-phenylmethyl, W is N, and Q is 2-amino-pyridin-3-ylmethyl-amino;
a compound of Formula (I) wherein A₁ is 4-trifluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-ethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-nitro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH(allyl), D is 4-methoxy-phenylmethyl, W is N, and Q is 2-amino-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-methoxy-phenyl, L₁ is CH₂, D is 4-aminocarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-pyridin-3-ylmethyl-amino;
a compound of Formula (I) wherein A₁ is 4-hydroxy-phenyl, L₁ is CH₂, D is 4-methoxy-phenylmethyl, W is N, and Q is 2-amino-pyridin-3-ylmethyl-amino;
a compound of Formula (I) wherein A₁ is 3-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-5-phenyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4-methoxy-pyridin-3-ylmethyl-amino;
a compound of Formula (I) wherein A₁ is 4-methoxy-phenyl, L₁ is CH₂, D is 4-methoxy-phenylmethyl, W is N, and Q is 6-methyl-pyridin-3-ylmethyl-amino;
a compound of Formula (I) wherein A₁ is 4-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-2-ylmethyl-amino;
a compound of Formula (I) wherein A₁ is 4-methoxy-phenyl, L₁ is CH₂, D is 4-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-trifluoromethyl-pyridin-2-ylmethyl-amino;
a compound of Formula (I) wherein A₁ is 4-methoxy-phenyl, L₁ is CH₂, D is 4-methoxy-phenylmethyl, W is N, and Q is 3-methyl-pyridin-2-ylmethyl-amino;
a compound of Formula (I) wherein A₁ is 4-methoxy-phenyl, L₁ is CH₂, D is 4-methoxy-phenylmethyl, W is N, and Q is 2-(2-methylthio-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(3-methyl-butylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(tetrahydro-furan-2-ylmethyl)-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(furan-2-ylmethyl-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-ethyl-pyrrolidin-2-ylmethyl-amino)-pyridin-3-ylmethyl-amino;
a compound of Formula (I) wherein A₁ is phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is phenoxy, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-nitro-phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methylthio-phenylmethyl, W is N, and Q is 2-amino-pyridin-3-ylmethyl-amino;
a compound of Formula (I) wherein A₁ is 4-methoxy-phenyl, L₁ is CH₂, D is pyridin-4-ylmethyl, W is N, and Q is 2-amino-pyridin-3-ylmethyl-amino;
a compound of Formula (I) wherein A₁ is 4-methoxy-phenyl, L₁ is CH₂, D is benzofuran-2-ylmethyl, W is N, and Q is 2-amino-pyridin-3-ylmethyl-amino;
a compound of Formula (I) wherein A₁ is 4-methoxy-phenyl, L₁ is CH₂, D is 5-methoxy-n-pentyl, W is N, and Q is 2-amino-pyridin-3-ylmethyl-amino;
a compound of Formula (I) wherein A₁ is 4-methoxy-phenyl, L₁ is CH₂, D is n-hexyl, W is N, and Q is 2-amino-pyridin-3-ylmethyl-amino;
a compound of Formula (I) wherein A₁ is 4-methoxy-phenyl, L₁ is CH₂, D is 3-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-cyano-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-cyano-phenylmethyl, W is N, and Q is 2-amino-pyridin-3-ylmethyl-amino;
a compound of Formula (I) wherein A₁ is 4-iodo-phenyl, L₁ is CH₂, D is 4-methoxy-phenylmethyl, W is N, and Q is 2-amino-pyridin-3-ylmethyl-amino;
a compound of Formula (I) wherein A₁ is 4-pyrazol-1-yl-phenyl, L₁ is CH₂, D is 4-methoxy-phenylmethyl, W is N, and Q is 2-amino-pyridin-3-ylmethyl-amino;
a compound of Formula (I) wherein A₁ is 4-fluoro-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 2-(4-methoxy-phenyl)-ethyl, W is N, and Q is 2-amino-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 2-amino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methylthio-phenylmethyl, W is N, and Q is 4,6-dimethyl-pyridin-3-ylmethyl-amino;
a compound of Formula (I) wherein A₁ is 4-methoxy-phenyl, L₁ is CH₂, D is pyridin-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-2-ylmethyl, W is N, and Q is 4,6-dimethyl-pyridin-3-ylmethyl-amino;
a compound of Formula (I) wherein A₁ is 4-methoxy-phenyl, L₁ is CH₂, D is n-hexyl, W is N, and Q is 4,6-dimethyl-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 4,6-dimethyl-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-methoxy-phenylmethyl, W is N, and Q is 4,6-dimethyl-pyridin-3-ylmethyl-amino;
a compound of Formula (I) wherein A₁ is 4-ethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-nitro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH(allyl), D is 4-methoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is pyridin-4-ylmethyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-amino-pyridin-2-ylmethyl-amino;
a compound of Formula (I) wherein A₁ is 4-methoxy-phenyl, L₁ is CH₂, D is 4-fluoro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-chloro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-4,6-dimethyl-pyridin-3-ylmethyl-amino;
a compound of Formula (I) wherein A₁ is indol-3-yl, L₁ is CH₂CH₂, D is 4-methoxy-phenylmethyl, W is N, and Q is 2-amino-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethoxy;
a compound of Formula (I) wherein A₁ is 4-methoxy-phenyl, L₁ is CH₂, D is 4-methoxy-phenylmethyl, W is N, and Q is 6-trifluoromethyl-pyridin-3-ylmethyl-amino;
a compound of Formula (I) wherein A₁ is 2,3-dihydro-benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-nitro-4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is indol-5-ylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is indol-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-t-butoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzothiophen-5-ylmethyl, W is N, and Q is 2-amino-pyridin-3-ylmethyl-amino;
a compound of Formula (I) wherein A₁ is 4-fluoro-phenoxy, L₁ is CH₂CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is benzothiophen-5-ylmethyl, W is N, and Q is 4,6-dimethyl-pyridin-3-ylmethyl-amino;
a compound of Formula (I) wherein A₁ is 2-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 2-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzothiophen-5-yl, L₁ is CH₂, D is 4-methoxy-phenylmethyl, W is N, and Q is 2-amino-pyridin-3-ylmethyl-amino;
a compound of Formula (I) wherein A₁ is benzothiophen-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-n-propylamino-pyridin-2-ylmethyl-amino;
a compound of Formula (I) wherein A₁ is 4-methoxy-phenyl, L₁ is CH₂, D is 4-methoxy-phenylmethyl, W is CH, and Q is 6-amino-pyridin-2-ylmethyl-amino;
a compound of Formula (I) wherein A₁ is 4-methoxy-phenyl, L₁ is CH₂, D is 4-methoxy-cyclohexylmethyl, W is N, and Q is 2-amino-pyridin-3-ylmethyl-amino;
a compound of Formula (I) wherein A₁ is 4-methoxy-phenyl, L₁ is CH₂, D is 4-methoxy-cyclohexylmethyl, W is N, and Q is 4,6-dimethyl-pyridin-3-ylmethyl-amino;
a compound of Formula (I) wherein A₁ is 4-methoxy-phenyl, L₁ is CH₂, D is 3,4-dichloro-phenylmethyl, W is N, and Q is 2-amino-pyridin-3-ylmethyl-amino;
a compound of Formula (I) wherein A₁ is 4-methoxy-phenyl, L₁ is CH₂, D is 4-(isoindol-1,3-dione-2-yl)-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-methoxycarbonyl-n-propyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-pyridin-2-yl-ethylamino;
a compound of Formula (I) wherein A₁ is 4-methoxy-phenyl, L₁ is CH₂, D is indol-4-ylmethyl, W is N, and Q is 2-amino-4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 6-amino-pyridin-2-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-pyrazol-1-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-iodo-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-[1,2,3]thiadiazol-4-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-3-yl-ethyl;
a compound of Formula (I) wherein A₁ is 4-methoxy-phenyl, L₁ is CH₂, D is indol-6-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzothiophen-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-hydroxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methylcarbonyloxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenyl, W is CH, and Q is 2-pyridin-4-yl-ethyl;
a compound of Formula (I) wherein A₁ is 4-methoxy-phenyl, L₁ is CH₂, D is 4-methoxy-phenyl, W is CH, and Q is cis-2-pyridin-4-yl-vinyl;
a compound of Formula (I) wherein A₁ is 2,3-dihydro-benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-2-yl-ethyl;
a compound of Formula (I) wherein A₁ is 4-methoxy-phenyl, L₁ is CH₂, D is 4-methoxy-phenylmethyl, W is N, and Q is imidazo[1,2-a]pyridin-8-ylmethyl-amino;
a compound of Formula (I) wherein A₁ is 4-methoxy-phenyl, L₁ is CH₂, D is 4-methoxy-phenylmethyl, W is CH, and Q is 2-(2-aminocarbonyl-pyridin-3-yl)-ethyl;
a compound of Formula (I) wherein A₁ is 4-methoxy-phenyl, L₁ is CH₂, D is 4-methoxy-phenylmethyl, W is CH, and Q is 2-amino-pyridin-3-ylmethoxy;
a compound of Formula (I) wherein A₁ is 4-hydroxymethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-aminocarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,6-difluoro-4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-methoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(2-amino-pyridin-3-yl)-ethyl;
a compound of Formula (I) wherein A₁ is 4-methoxy-phenyl, L₁ is CH₂, D is 2,4-dimethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-4,6-dimethyl-pyridin-3-ylmethoxy;
a compound of Formula (I) wherein A₁ is 4-methoxy-phenyl, L₁ is CH₂, D is 3-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-fluoro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-methoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethylthio;
a compound of Formula (I) wherein A₁ is 4-methoxy-phenyl, L₁ is CH₂, D is 2-methyl-2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-piperidinyl)-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(4-amino-pyridin-3-yl)-ethyl;
a compound of Formula (I) wherein A₁ is 4-methoxy-phenyl, L₁ is CH₂, D is 4-methoxy-phenylmethyl, W is N, and Q is 2-(pyridin-4-yl)-ethylamino;
a compound of Formula (I) wherein A₁ is 1-methyl-1H-benzotriazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(6-amino-pyridin-2-yl)ethyl;
a compound of Formula (I) wherein A₁ is 4-methoxy-phenyl, L₁ is CH₂, D is 5-methoxy-n-pentyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 1-(2-amino-pyridin-4-yl)-ethoxy;
a compound of Formula (I) wherein A₁ is 2,3-dihydro-benzofuran-5-yl, L₁ is CH₂, D is 2,3-dihydro-benzofuran-5-ylmethyl, W is N, and Q is N-oxo-2-amino-4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 2-amino-pyridin-3-ylmethyl-amino;
a compound of Formula (I) wherein A₁ is indol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyrimidin-4-ylmethoxy;
and
a compound of Formula (I) wherein A₁ is 2,3-dihydro-benzofuran-5-yl, L₁ is CH₂, D is 4-difluoromethoxy-phenylmethyl, W is N, and Q is N-oxo-2-amino-4,6-dimethyl-pyridin-3-ylmethyl-amino

12. A method as in claim 11 wherein the pain is inflammatory pain.

13. A method as in claim 11 wherein the pain is visceral pain.

14. A method as in claim 11 wherein the pain is acute pain.

15. The use of a compound as in claim 11 for the preparation of a medicament or pharmaceutical composition for the treatment of inflammatory pain, visceral pain, or acute pain, in a subject in need thereof.

16. A method of treating or preventing pain comprising administering to a subject in need thereof, a therapeutically effective amount of a pharmaceutical composition comprising a compound of Formula (I) 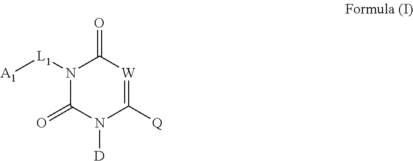
or an enantiomer, diastereomer, solvate, or pharmaceutically acceptable salt thereof;
wherein:
A₁ is CF₃, C₁₋₄alkoxy, aryl, aryloxy, benzofused heterocyclyl, or heteroaryl; wherein aryl, aryloxy, and heteroaryl are optionally substituted with pyrazol-1-yl or [1,2,3]thiadiazol-4-yl; or aryl, aryloxy, the benzo portion of benzofused heterocyclyl, and heteroaryl are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl; provided that A₁ is other than 3,5-di-t-butyl-phenyl;
L₁ is —(CH₂)ᵣ—, —CH₂C₂₋₄alkenyl-, or —CH₂CH₂X(CH₂)ₛ—, wherein L₁ is optionally substituted with one to two substituents independently selected from the group consisting of C₁₋₆alkyl, C₂₋₆alkenyl, C₂₋₆alkynyl, and halogen; and, r is an integer of 1 to 5; such that r is greater than or equal to 4 when A₁ is C₁₋₄alkoxy;
s is an integer of 1 to 3;
X is O or S;
D is —P-A₂;
wherein P is —(CH₂)₁₋₂— or —CH₂CH═CH— when A₂ is phenyl, benzofused heterocyclyl, heteroaryl, or C₃₋₈cycloalkyl; alternatively, P is —(CH₂)₃₋₆—, when A₂ is hydrogen, C₁₋₄alkoxy, or C₁₋₄alkoxycarbonyl; and wherein P is optionally substituted with one to two substituents independently selected from the group consisting of C₁₋₆alkyl, C₂₋₆alkenyl, C₂₋₆alkynyl, and halogen;
A₂ is hydrogen, C₁₋₄alkoxy, C₁₋₄alkoxycarbonyl, phenyl, benzofused heterocyclyl, heteroaryl, tetrahydro-pyranyl, piperidinyl, or C₃₋₈cycloalkyl; wherein phenyl, heteroaryl, the benzo portion of benzofused heterocyclyl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, N-isoindole-1,3-dione, C₁₋₆alkylthio, C₁₋₆alkylsulfonyl, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; such that no more than two substituents on A₂ are aryl(C₁₋₆)alkoxy, phenyl, N-isoindole-1,3-dione, or a non fused C₃₋₆cycloalkyloxy;
provided that A₂ is other than 3,5-di-t-butyl-phenyl;
W is N or C(R_{W}); wherein R_{W} is H or C₁₋₂alkyl;
Q is selected from the group consisting of (a) to (g), wherein
(a) is —NH(CH₂)₂—Ar₁ wherein Ar₁ is pyridinyl optionally substituted with one to three C₁₋₄alkyl substituents or a substituent selected from the group consisting of C₁₋₄alkoxy and amino;
provided that when Ar₁ is an unsubstituted pyridin-3-yl or unsubstituted pyridin-4-yl, and A₂ is 4-methoxy-phenyl, A₁ is other than unsubstituted phenyl or 3,4-dichloro-phenyl;
(b) is —NHCH(R_{z})-Ar₂ wherein R_{z} is H or C₁₋₃alkyl; Ar₂ is pyridinyl, pyrimidinyl, pyrazinyl, 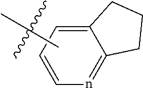
1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; and wherein Ar₂ is optionally substituted with one to three substituents independently selected from the group consisting of C₁₋₄alkyl, trifluoromethyl, hydroxyl-C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄alkoxy, C₃₋₈cycloalkylamino, amino, (C₁₋₆alkyl)amino, and di(C₁₋₆alkyl)amino; or Ar₂ is optionally substituted with one amino group and three substituents independently selected from the group consisting of C₁₋₄alkyl and C₁₋₄alkoxy;
wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, C₁₋₄alkylthio, hydroxy, a 5 to 6 membered heteroaryl, or a 5 to 6 membered heterocyclyl; wherein a nitrogen atom of the 5 to 6 membered heterocyclyl is optionally substituted with a C₁₋₄alkyl substituent;
and wherein pyridin-2-yl and pyridin-3-yl are optionally further substituted with N-pyrrolidinyl, N-piperazinyl, N-piperidinyl, N-morpholinyl, N-thiomorpholinyl, —CH₂—O—CH₂—PH, and phenyl; wherein the phenyl substituent of pyridin-2-yl and pyridin-3-yl is optionally substituted with one to three substituents independently selected from the group consisting of C₁₋₄alkyl, C₁₋₄alkoxy, and halogen;
provided that when Q is —NHCH₂(2-amino-pyridin-3-yl), and A₁ is pyridin-4-yl, 4-C₁₋₆alkyl-phenyl, 3,4-dichloro-phenyl, or 4-methanesulfonyl-phenyl, A₂ is other than 4-methoxy-phenyl;
provided that when Q is —NHCH₂(2-amino-pyridin-3-yl), L₁ is —(CH₂)₂— or —(CH₂)₅—, and A₁ is methoxy, A₂ is other than 4-difluoromethoxy-phenyl or 4-methoxy-phenyl;
provided that when Q is —NHCH₂(2-amino-pyridin-3-yl), and A₁ is benzotriazol-1-yl, A₂ is other than 4-difluoromethoxy-phenyl;
provided that when Q is —NHCH₂(2-amino-pyridin-3-yl), L₁ is —(CH₂)₃—, and A₁ is pyrrol-1-yl, A₂ is other than 4-methoxy-phenyl;
provided that when Q is —NHCH₂(2-amino-pyridin-3-yl), L₁ is —(CH₂)₂—, and A₁ is 4-nitro-phenyl or ethoxy, A₂ is other than 4-methoxy-phenyl;
provided that when Q is —NHCH₂(2-amino-pyridin-3-yl), and A₁ is 4-fluoro-phenyl, A₂ is other than 4-fluoro-phenyl;
provided that when Q is —NHCH₂(6-amino-pyridin-2-yl), and A₁ is 4-fluoro-phenyl, A₂ is other than 4-trifluoromethoxy-phenyl;
provided that when Q is —NHCH₂(6-methyl-pyridin-2-yl), and A₁ is 4-methoxy-phenyl, A₂ is other than 4-methoxy-phenyl;
provided that when Q is —NHCH₂(imidazo[1,2-a]pyridinyl), and A₁ is 4-fluoro-phenyl, A₂ is other than 4-methoxy-phenyl;
provided that when Q is —NHCH₂(pyridin-4-yl), and A₁ is unsubstituted phenyl or 3,4-dichloro-phenyl, A₂ is other than 4-methoxy-phenyl;
provided that when Q is —NHCH₂(4,6-dimethyl-pyridin-3-yl), and A₁ is 4-methoxy-phenyl, —P-A₂ is other than —(CH₂)₅-methoxy;
provided that when Q is —NHCH₂(4,6-dimethyl-pyridin-3-yl), L₁ is —(CH₂)₂—, and A₁ is pyrazol-1-yl, A₂ is other than 4-difluoromethoxy-phenyl;
provided that when Q is —NHCH₂(4,6-dimethyl-pyridin-3-yl) and A₁ is 4-methoxy-phenyl, A₂ is other than 2-ethyl-phenyl, 4-ethyl-phenyl, 3-methoxy-phenyl, 3-cyano-phenyl, 3-nitro-phenyl, and 3-trifluoromethyl-4-nitro-phenyl;
provided that when Q is —NHCH₂(4,6-dimethyl-pyridin-3-yl) and A₁ is quinolin-8-yl, benzotriazol-1-yl, 3,5-dimethyl-pyrazolyl, 2-fluoro-phenyl, 2-chloro-phenyl, 2-nitro-phenyl, 2-trifluoromethyl-phenyl, 2-difluoromethoxy-phenyl, 3-difluoromethoxy-phenyl, 2-trifluoromethoxy-phenyl, 2,4-difluoro-phenyl, 2,6-difluoro-phenyl, 2,6-dichloro-phenyl, 2-chloro-4-fluoro-phenyl, 2,6-difluoro-4-methoxy-phenyl, or 4-trifluoromethoxy-phenyl, A₂ is other than 4-difluoromethoxy-phenyl;
and, provided that when Q is —NHCH₂(4,6-dimethyl-pyridin-3-yl) and A₁ is 3-nitro-4-methoxy-phenyl, 2,6-difluoro-4-methoxy-phenyl, or 3,4-dichloro-phenyl, A₂ is other than 4-methoxy-phenyl;
(c) is —CH₂NHCH₂—Ar₃, wherein W is N or CH, and Ar₃ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and that the point of attachment to quinolinyl is at the 2, 3, or 4-position; wherein Ar₃ is optionally substituted with one to three substituents independently selected from the group consisting of C₁₋₄alkyl, amino(C₁₋₄alkyl, (C₁₋₄alkyl)amino-(C₁₋₄alkyl, di(C₁₋₄alkyl)amino-(C₁₋₄alkyl, C₁₋₄alkoxy, amino, (C₁₋₆alkyl)amino, and di(C₁₋₆alkyl)amino;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(d) is —(CH₂)₂—Ar₄, wherein Ar₄ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₄ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄-alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(e) is —CH═CH—Ar₅; wherein Ar₅ is pyridinyl, pyrimidinyl, 1,2,3,4-tetrahydro-[1,8]naphthyridinyl, imidazo[1,2-a]pyridinyl, or quinolinyl; such that the point of attachment to 1,2,3,4-tetrahydro-[1,8]naphthyridinyl is at the 6 or 7 position, and the point of attachment to quinolinyl is at the 2, 3, or 4-position; wherein Ar₅ is optionally substituted with one to three substituents independently selected from the group consisting of C₁₋₄alkyl, amino(C₁₋₄alkyl, (C₁₋₄alkyl)amino-(C₁₋₄)alkyl, di(C₁₋₄alkyl)amino-(C₁₋₄alkyl, C₁₋₄-alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
(f) is —O—CH(R₁)—Ar₆ when W is CH; or, (f) is —S—CH(R₁)—Ar₆ and W is N or CH; wherein R₁ is hydrogen or C₁₋₄alkyl, and Ar₆ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;
wherein Ar₆ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
provided that when Q is —O—CH(R₁)—Ar₆, A₁ and A₂ are 4-methoxy-phenyl, and R₁ is hydrogen, Ar₆ is other than unsubstituted pyridin-2-yl or 2-amino-pyridin-4-yl;
and
(g) is —X₁—(CH(Rₓ)₂—Ar₇ when W is CH; wherein X₁ is O or S, Rₓ is H or C₁₋₄alkyl, and Ar₇ is pyridinyl, pyrimidinyl, 1,2,3,4-tetrahydro-[1,8]naphthyridinyl, imidazo[1,2-a]pyridinyl, or quinolinyl such that the point of attachment to 1,2,3,4-tetrahydro-[1,8]naphthyridinyl is at the 6 or 7 position, and the point of attachment to quinolinyl is at the 2, 3, or 4-position;
wherein Ar₇ is optionally substituted with one to three substituents independently selected from the group consisting of C₁₋₄alkyl, amino(C₁₋₄)alkyl, (C₁₋₄alkyl)amino-(C₁₋₄)alkyl, di(C₁₋₄alkyl)amino-(C₁₋₄alkyl, C₁₋₄-alkoxy, amino, (C₁₋₆alkyl)amino, di(C₁₋₆alkyl)amino, halogen, and aminocarbonyl;
and wherein the C₁₋₆alkyl group of (C₁₋₆alkyl)amino and di(C₁₋₆alkyl)amino is optionally substituted with amino, (C₁₋₄alkyl)amino, di(C₁₋₄alkyl)amino, C₃₋₈cycloalkylamino, C₁₋₄alkoxy, or hydroxy;
provided that when Q is —O (CH₂)₂—Ar₇ and A₁ and A₂ are 4-methoxy-phenyl, Ar₇ is other than unsubstituted pyridin-2-yl or unsubstituted pyridin-3-yl;
wherein a nitrogen atom of Ar₁, Ar₂, Ar₃, Ar₄, Ar₅, Ar₆, and Ar₇ is optionally substituted with oxo.

17. A method as in claim 16 wherein the pain is inflammatory pain.

18. A method as in claim 16 wherein the pain is visceral pain.

19. A method as in claim 16 wherein the pain is acute pain.

20. A method of treating or preventing pain comprising administering to a subject in need thereof, a therapeutically effective amount of a pharmaceutical composition comprising a compound of Formula (I) 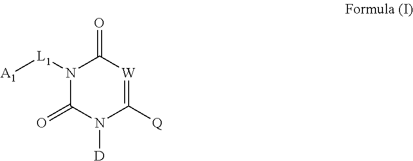
or an enantiomer, diastereomer, solvate, or pharmaceutically acceptable salt thereof;
wherein:
A₁ is substituted phenyl, benzotriazolyl, benzofuranyl, benzo[1,3]dioxalyl or 2,3-dihydro-benzofuranyl; wherein phenyl is substituted at the 4-position with methoxy, fluoro, or methylthio; and wherein A₁ other than substituted phenyl is optionally substituted with one to two substituents independently selected from the group consisting of methyl, methoxy, fluoro and methylthio;
L₁ is —CH₂—;
D is —P-A₂; wherein P is —CH₂— when A₂ is phenyl, 2,3-dihydro-benzofuranyl, indolyl, benzofuranyl, pyridin-3-yl, or benzothiophenyl; alternatively, P is —(CH₂)₄₋₆—, when A₂ is C₁₋₄alkoxy;
A₂ is C₁₋₄alkoxy, phenyl, 2,3-dihydro-benzofuranyl, indolyl, benzofuranyl, pyridin-3-yl, or benzothiophenyl; wherein A₂ other than C₁₋₄alkoxy is optionally substituted with one to two substituents independently selected from the group consisting of C₁₋₄alkoxy, fluoro, fluorinated C₁₋₄alkoxy, C₁₋₄alkylthio, C₁₋₄alkylsulfonyl, C₁₋₄alkoxycarbonyl, nitro, and hydroxy;
W is N;
Q is —NHCH₂—Ar₂ wherein Ar₂ is unsubstituted pyridin-2-yl, 4,6-dimethyl-pyridin-3-yl, 2-amino-pyridin-3-yl, or 2-((C₁₋₄alkyl)amino)-pyridin-3-yl; wherein the C₁₋₄alkyl group of (C₁₋₄alkyl)amino is optionally substituted with di(C₁₋₄alkyl)amino, C₁₋₄alkoxy, or hydroxy; and wherein 2-amino-pyridin-3-yl is optionally further substituted with 4,6-dimethyl or 4-methoxy;
provided that when Q is —NHCH₂(2-amino-pyridin-3-yl), and A₁ is pyridin-4-yl or 4-methyl-phenyl, A₂ is other than 4-methoxy-phenyl;
provided that when Q is —NHCH₂(2-amino-pyridin-3-yl), and A₁ is benzotriazol-1-yl, A₂ is other than 4-difluoromethoxy-phenyl;
provided that when Q is —NHCH₂(2-amino-pyridin-3-yl), and A₁ is 4-fluoro-phenyl, A₂ is other than 4-fluoro-phenyl;
provided that when Q is —NHCH₂(6-amino-pyridin-2-yl), and A₁ is 4-fluoro-phenyl, A₂ is other than 4-trifluoromethoxy-phenyl;
provided that when Q is —NHCH₂(6-methyl-pyridin-2-yl), and A₁ is 4-methoxy-phenyl, A₂ is other than 4-methoxy-phenyl;
provided that when Q is —NHCH₂(imidazo[1,2-a]pyridinyl), and A₁ is 4-fluoro-phenyl, A₂ is other than 4-methoxy-phenyl;
provided that when Q is —NHCH₂(4,6-dimethyl-pyridin-3-yl), and A₁ is 4-methoxy-phenyl, —P-A₂ is other than —(CH₂)₅-methoxy;
provided that when Q is —NHCH₂(4,6-dimethyl-pyridin-3-yl) and A₁ is 4-methoxy-phenyl, A₂ is other than 3-methoxy-phenyl or 3-nitro-phenyl;
and
provided that when Q is —NHCH₂(4,6-dimethyl-pyridin-3-yl) and A₁ is benzotriazol-1-yl, A₂ is other than 4-difluoromethoxy-phenyl;
wherein a nitrogen atom of Ar₂ and Ar₄ is optionally substituted with oxo.

21. A method of treating or preventing pain comprising administering to a subject in need thereof, a therapeutically effective amount of a pharmaceutical composition comprising a compound of Formula (I) 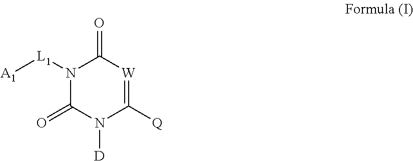
or pharmaceutically acceptable salt thereof;
selected from the group consisting of
a compound of Formula (I) wherein A₁ is 4-methoxy-phenyl, L₁ is CH₂, D is 4-methoxy-phenylmethyl, W is N, and Q is 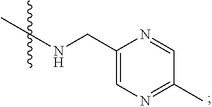
a compound of Formula (I) wherein A₁ is 4-methoxy-phenyl, L₁ is CH₂, D is 4-methoxy-phenylmethyl, W is N, and Q is 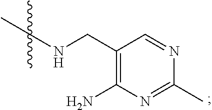
a compound of Formula (I) wherein A₁ is 4-chloro-phenyl, L₁ is CH₂, D is —(CH₂)₅OCH₃, W is N, and Q is 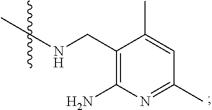
a compound of Formula (I) wherein A₁ is 3,4-dichloro-phenyl, L₁ is CH₂, D is 4-methoxy-phenylmethyl, W is N, and Q is 2-(pyridin-2-yl)ethyl-amino;
a compound of Formula (I) wherein A₁ is 3,4-dichloro-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 5-amino-pyridin-2-ylmethyl-amino;
a compound of Formula (I) wherein A₁ is 4-chloro-phenyl, L₁ is CH₂, D is 4-methoxy-phenylmethyl, W is N, and Q is 6-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-amino-pyrimidin-5-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-aminomethyl;
a compound of Formula (I) wherein A₁ is 4-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-quinolin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-pyridin-3-yl)-ethylamino;
a compound of Formula (I) wherein A₁ is 4-fluoro-phenyl, L₁ is CH₂, D is 4-methoxy-phenylmethyl, W is N, and Q is 2-N-pyrrolidinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperazinyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-piperidinyl-pyridin-3-ylmethyl-amino;
a compound of Formula (I) wherein A₁ is 4-fluoro-phenyl, L₁ is CH₂, D is 4-methoxy-phenylmethyl, W is N, and Q is 2-methylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-n-butylamino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-N-thiomorpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-ethylamino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-N-morpholino-pyridin-3-ylmethyl-amino;
a compound of Formula (I) wherein A₁ is 4-fluoro-phenyl, L₁ is CH₂, D is 4-methoxy-phenylmethyl, W is N, and Q is 1,2,3,4-tetrahydro-[1,8]naphthyridin-7-ylmethyl-amino;
a compound of Formula (I) wherein A₁ is 4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-2-yl, L₁ is CH₂, D is 4-methoxy-phenylmethyl, W is N, and Q is 2-amino-pyridin-3-ylmethyl-amino;
a compound of Formula (I) wherein A₁ is 4-methylthio-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-(4-fluoro-phenyl)-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-hydroxy-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-(2-amino-ethylamino)-pyridin-3-ylmethyl-amino;
a compound of Formula (I) wherein A₁ is 4-fluoro-phenyl, L₁ is CH₂, D is 4-methoxy-phenylmethyl, W is N, and Q is 2-cyclohexylamino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-2-amino-pyridin-3-ylmethyl-amino;
a compound of Formula (I) wherein A₁ is 4-methoxy-phenyl, L₁ is CH₂, D is 4-hydroxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-n-propylamino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methylcarbonylamino-phenylmethyl, W is N, and Q is 2-amino-pyridin-3-ylmethyl-amino;
a compound of Formula (I) wherein A₁ is 4-methoxy-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-2-ylmethyl-amino;
a compound of Formula (I) wherein A₁ is 4-methoxy-phenyl, L₁ is CH₂, D is 4-methoxy-phenylmethyl, W is N, and Q is pyridin-3-ylmethyl-amino;
a compound of Formula (I) wherein A₁ is 4-methoxy-phenyl, L₁ is CH₂, D is 4-methoxy-phenylmethyl, W is N, and Q is pyridin-4-ylmethyl-amino;
a compound of Formula (I) wherein A₁ is 3-methoxy-phenyl, L₁ is CH₂, D is 4-methoxy-phenylmethyl, W is N, and Q is 2-amino-pyridin-3-ylmethyl-amino;
a compound of Formula (I) wherein A₁ is phenyl, L₁ is CH₂, D is 4-methoxy-phenylmethyl, W is N, and Q is 2-amino-pyridin-3-ylmethyl-amino;
a compound of Formula (I) wherein A₁ is 4-cyano-phenyl, L₁ is CH₂, D is 4-methoxy-phenylmethyl, W is N, and Q is 2-amino-pyridin-3-ylmethyl-amino;
a compound of Formula (I) wherein A₁ is 4-trifluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-ethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-nitro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH(allyl), D is 4-methoxy-phenylmethyl, W is N, and Q is 2-amino-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(2-dimethylamino-ethylamino)-pyridin-3-ylmethyl-amino;
a compound of Formula (I) wherein A₁ is 4-methoxy-phenyl, L₁ is CH₂, D is 4-aminocarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-pyridin-3-ylmethyl-amino;
a compound of Formula (I) wherein A₁ is 4-hydroxy-phenyl, L₁ is CH₂, D is 4-methoxy-phenylmethyl, W is N, and Q is 2-amino-pyridin-3-ylmethyl-amino;
a compound of Formula (I) wherein A₁ is 3-fluoro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-5-phenyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4-methoxy-pyridin-3-ylmethyl-amino;
a compound of Formula (I) wherein A₁ is 4-methoxy-phenyl, L₁ is CH₂, D is 4-methoxy-phenylmethyl, W is N, and Q is 6-methyl-pyridin-3-ylmethyl-amino;
a compound of Formula (I) wherein A₁ is 4-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-2-ylmethyl-amino;
a compound of Formula (I) wherein A₁ is 4-methoxy-phenyl, L₁ is CH₂, D is 4-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 6-trifluoromethyl-pyridin-2-ylmethyl-amino;
a compound of Formula (I) wherein A₁ is 4-methoxy-phenyl, L₁ is CH₂, D is 4-methoxy-phenylmethyl, W is N, and Q is 3-methyl-pyridin-2-ylmethyl-amino;
a compound of Formula (I) wherein A₁ is 4-methoxy-phenyl, L₁ is CH₂, D is 4-methoxy-phenylmethyl, W is N, and Q is 2-(2-methylthio-ethylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(3-methyl-butylamino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(tetrahydro-furan-2-ylmethyl)-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(furan-2-ylmethyl-amino)-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-ethyl-pyrrolidin-2-ylmethyl-amino)-pyridin-3-ylmethyl-amino;
a compound of Formula (I) wherein A₁ is phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is phenoxy, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-(2-methoxy-ethylamino)-pyridin-3-ylmethyl-amino;
a compound of Formula (I) wherein A₁ is 4-nitro-phenyl, L₁ is CH₂CH₂, D is 4-methoxy-phenylmethyl, W is N, and Q is 2-(2-methoxy-ethylamino)-pyridin-3-ylmethyl-amino;
a compound of Formula (I) wherein A₁ is 4-methoxy-phenyl, L₁ is CH₂, D is 4-methylthio-phenylmethyl, W is N, and Q is 2-amino-pyridin-3-ylmethyl-amino;
a compound of Formula (I) wherein A₁ is 4-methoxy-phenyl, L₁ is CH₂, D is pyridin-4-ylmethyl, W is N, and Q is 2-amino-pyridin-3-ylmethyl-amino;
a compound of Formula (I) wherein A₁ is 4-methoxy-phenyl, L₁ is CH₂, D is benzofuran-2-ylmethyl, W is N, and Q is 2-amino-pyridin-3-ylmethyl-amino;
a compound of Formula (I) wherein A₁ is 4-methoxy-phenyl, L₁ is CH₂, D is 5-methoxy-n-pentyl, W is N, and Q is 2-amino-pyridin-3-ylmethyl-amino;
a compound of Formula (I) wherein A₁ is 4-methoxy-phenyl, L₁ is CH₂, D is n-hexyl, W is N, and Q is 2-amino-pyridin-3-ylmethyl-amino;
a compound of Formula (I) wherein A₁ is 4-methoxy-phenyl, L₁ is CH₂, D is 3-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-cyano-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-ethyl-phenylmethyl, W is N, and Q is 2-amino-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-cyano-phenylmethyl, W is N, and Q is 2-amino-pyridin-3-ylmethyl-amino;
a compound of Formula (I) wherein A₁ is 4-iodo-phenyl, L₁ is CH₂, D is 4-methoxy-phenylmethyl, W is N, and Q is 2-amino-pyridin-3-ylmethyl-amino;
a compound of Formula (I) wherein A₁ is 4-pyrazol-1-yl-phenyl, L₁ is CH₂, D is 4-methoxy-phenylmethyl, W is N, and Q is 2-amino-pyridin-3-ylmethyl-amino;
a compound of Formula (I) wherein A₁ is 4-fluoro-phenyl, L₁ is CH₂, D is 4-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-methoxycarbonyl-phenylmethyl, W is N, and Q is 2-amino-pyridin-3-ylmethyl-amino;
a compound of Formula (I) wherein A₁ is 4-methoxy-phenyl, L₁ is CH₂, D is 2-(4-methoxy-phenyl)-ethyl, W is N, and Q is 2-amino-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 2-amino-pyridin-3-ylmethyl-amino;
a compound of Formula (I) wherein A₁ is 4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methylthio-phenylmethyl, W is N, and Q is 4,6-dimethyl-pyridin-3-ylmethyl-amino;
a compound of Formula (I) wherein A₁ is 4-methoxy-phenyl, L₁ is CH₂, D is pyridin-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-2-ylmethyl, W is N, and Q is 4,6-dimethyl-pyridin-3-ylmethyl-amino;
a compound of Formula (I) wherein A₁ is 4-methoxy-phenyl, L₁ is CH₂, D is n-hexyl, W is N, and Q is 4,6-dimethyl-pyridin-3-ylmethyl-amino;
a compound of Formula (I) wherein A₁ is 4-methoxy-phenyl, L₁ is CH₂, D is 6-methoxy-pyridin-3-ylmethyl, W is N, and Q is 4,6-dimethyl-pyridin-3-ylmethyl-amino;
a compound of Formula (I) wherein A₁ is 4-methoxy-phenyl, L₁ is CH₂, D is 2-trifluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-methoxy-phenylmethyl, W is N, and Q is 4,6-dimethyl-pyridin-3-ylmethyl-amino;
a compound of Formula (I) wherein A₁ is 4-ethoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-nitro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH(allyl), D is 4-methoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-phenyl, L₁ is CH₂, D is 4-methoxy-phenylmethyl, W is N, and Q is 4,6-dimethyl-pyridin-3-ylmethyl-amino;
a compound of Formula (I) wherein A₁ is pyridin-4-ylmethyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-amino-pyridin-2-ylmethyl-amino;
a compound of Formula (I) wherein A₁ is 4-methoxy-phenyl, L₁ is CH₂, D is 4-fluoro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-chloro-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is N-oxo-4,6-dimethyl-pyridin-3-ylmethyl-amino;
a compound of Formula (I) wherein A₁ is indol-3-yl, L₁ is CH₂CH₂, D is 4-methoxy-phenylmethyl, W is N, and Q is 2-amino-pyridin-3-ylmethyl-amino;
a compound of Formula (I) wherein A₁ is 2,3-dihydro-benzo[1,4]dioxin-2-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethoxy;
a compound of Formula (I) wherein A₁ is 4-methoxy-phenyl, L₁ is CH₂, D is 4-methoxy-phenylmethyl, W is N, and Q is 6-trifluoromethyl-pyridin-3-ylmethyl-amino;
a compound of Formula (I) wherein A₁ is 2,3-dihydro-benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-nitro-4-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is benzofuran-5-ylmethyl, W is N, and Q is 2-amino-pyridin-3-ylmethyl-amino;
a compound of Formula (I) wherein A₁ is 4-methoxy-phenyl, L₁ is CH₂, D is indol-5-ylmethyl, W is N, and Q is 2-amino-pyridin-3-ylmethyl-amino;
a compound of Formula (I) wherein A₁ is 4-methoxy-phenyl, L₁ is CH₂, D is 2,3-dihydro-benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzofuran-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is indol-5-ylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 2-amino-pyridin-3-ylmethyl-amino;
a compound of Formula (I) wherein A₁ is 4-methoxy-phenyl, L₁ is CH₂, D is 4-methanesulfonyl-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-t-butoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-nitro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 2-amino-pyridin-3-ylmethyl-amino;
a compound of Formula (I) wherein A₁ is 4-methoxy-phenyl, L₁ is CH₂, D is indol-4-ylmethyl, W is N, and Q is 4,6-dimethyl-pyridin-3-ylmethyl-amino;
a compound of Formula (I) wherein A₁ is 4-methoxy-phenyl, L₁ is CH₂, D is benzothiophen-5-ylmethyl, W is N, and Q is 2-amino-pyridin-3-ylmethyl-amino;
a compound of Formula (I) wherein A₁ is 4-fluoro-phenoxy, L₁ is CH₂CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is benzothiophen-5-ylmethyl, W is N, and Q is 4,6-dimethyl-pyridin-3-ylmethyl-amino;
a compound of Formula (I) wherein A₁ is 2-methoxy-phenyl, L₁ is CH₂, D is 4-methoxy-phenylmethyl, W is N, and Q is 2-amino-pyridin-3-ylmethyl-amino;
a compound of Formula (I) wherein A₁ is 2-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzothiophen-5-yl, L₁ is CH₂, D is 4-methoxy-phenylmethyl, W is N, and Q is 2-amino-pyridin-3-ylmethyl-amino;
a compound of Formula (I) wherein A₁ is benzothiophen-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 6-n-propylamino-pyridin-2-ylmethyl-amino;
a compound of Formula (I) wherein A₁ is 4-methoxy-phenyl, L₁ is CH₂, D is 4-methoxy-phenylmethyl, W is CH, and Q is 6-amino-pyridin-2-ylmethyl-amino;
a compound of Formula (I) wherein A₁ is 4-methoxy-phenyl, L₁ is CH₂, D is 4-methoxy-cyclohexylmethyl, W is N, and Q is 2-amino-pyridin-3-ylmethyl-amino;
a compound of Formula (I) wherein A₁ is 4-methoxy-phenyl, L₁ is CH₂, D is 4-methoxy-cyclohexylmethyl, W is N, and Q is 4,6-dimethyl-pyridin-3-ylmethyl-amino;
a compound of Formula (I) wherein A₁ is 4-methoxy-phenyl, L₁ is CH₂, D is 3,4-dichloro-phenylmethyl, W is N, and Q is 2-amino-pyridin-3-ylmethyl-amino;
a compound of Formula (I) wherein A₁ is 4-methoxy-phenyl, L₁ is CH₂, D is 4-(isoindol-1,3-dione-2-yl)-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 3-methoxycarbonyl-n-propyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-pyridin-2-yl-ethylamino;
a compound of Formula (I) wherein A₁ is 4-methoxy-phenyl, L₁ is CH₂, D is indol-4-ylmethyl, W is N, and Q is 2-amino-4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 6-amino-pyridin-2-ylmethyl-amino;
a compound of Formula (I) wherein A₁ is 4-methoxy-phenyl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-pyrazol-1-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-iodo-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-trifluoromethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-difluoromethoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxycarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-fluoro-phenoxy, L₁ is CH₂CH₂, D is 4-difluoromethoxy-phenylmethyl, W is N, and Q is 4,6-dimethyl-pyridin-3-ylmethyl-amino;
a compound of Formula (I) wherein A₁ is 4-[1,2,3]thiadiazol-4-yl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-3-yl-ethyl;
a compound of Formula (I) wherein A₁ is 4-methoxy-phenyl, L₁ is CH₂, D is indol-6-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 2-amino-pyridin-3-ylmethyl-amino;
a compound of Formula (I) wherein A₁ is 4-methoxy-phenyl, L₁ is CH₂, D is indol-7-ylmethyl, W is N, and Q is 4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzothiophen-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 4-methylthio-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzofuran-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-cyano-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-hydroxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methylcarbonyloxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenyl, W is CH, and Q is 2-pyridin-4-yl-ethyl;
a compound of Formula (I) wherein A₁ is 4-methoxy-phenyl, L₁ is CH₂, D is 4-methoxy-phenyl, W is CH, and Q is cis-2-pyridin-4-yl-vinyl;
a compound of Formula (I) wherein A₁ is 2,3-dihydro-benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is benzofuran-5-yl, L₁ is CH₂, D is 2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-pyridin-2-yl-ethyl;
a compound of Formula (I) wherein A₁ is 4-methoxy-phenyl, L₁ is CH₂, D is 4-methoxy-phenylmethyl, W is N, and Q is imidazo[1,2-a]pyridin-8-ylmethyl-amino;
a compound of Formula (I) wherein A₁ is 4-methoxy-phenyl, L₁ is CH₂, D is 4-methoxy-phenylmethyl, W is CH, and Q is 2-(2-aminocarbonyl-pyridin-3-yl)-ethyl;
a compound of Formula (I) wherein A₁ is 4-methoxy-phenyl, L₁ is CH₂, D is 4-methoxy-phenylmethyl, W is CH, and Q is 2-amino-pyridin-3-ylmethoxy;
a compound of Formula (I) wherein A₁ is 4-hydroxymethyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-aminocarbonyl-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,6-difluoro-4-methoxy-phenyl, L₁ is CH₂, D is 4-difluoromethoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-methoxy-phenylmethyl, W is N, and Q is 4,6-dimethyl-pyridin-3-ylmethyl-amino;
a compound of Formula (I) wherein A₁ is methoxy, L₁ is (CH₂)₅, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(2-amino-pyridin-3-yl)-ethyl;
a compound of Formula (I) wherein A₁ is 4-methoxy-phenyl, L₁ is CH₂, D is 2,4-dimethoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is N, and Q is 4-methyl-pyridin-3-ylmethyl-amino;

22. a compound of Formula (I) wherein A₁ is 4-methoxy-phenyl, L₁ is CH₂, D is 4-methoxy-phenylmethyl, W is CH, and Q is 2-amino-4,6-dimethyl-pyridin-3-ylmethoxy;
a compound of Formula (I) wherein A₁ is 4-methoxy-phenyl, L₁ is CH₂, D is 3-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 3-fluoro-4-methoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 2-fluoro-4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-methoxy-phenylmethyl, W is N, and Q is 4,6-dimethyl-pyridin-3-ylmethyl-amino;
a compound of Formula (I) wherein A₁ is 2,3-dihydro-benzo[1,4]dioxin-6-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is pyridin-3-ylmethylthio;
a compound of Formula (I) wherein A₁ is 4-methoxy-phenyl, L₁ is CH₂, D is 2-methyl-2,3-dihydro-benzofuran-5-ylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is N, and Q is 2-(N-piperidinyl)-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(4-amino-pyridin-3-yl)-ethyl;
a compound of Formula (I) wherein A₁ is 4-methoxy-phenyl, L₁ is CH₂, D is 4-methoxy-phenylmethyl, W is N, and Q is 2-(pyridin-4-yl)-ethylamino;
a compound of Formula (I) wherein A₁ is 1-methyl-1H-benzotriazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is benzo[1,2,3]thiadiazol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is 3-fluoro-4-methoxy-phenyl, L₁ is CH₂, D is 4-methoxy-phenylmethyl, W is N, and Q is 4,6-dimethyl-pyridin-3-ylmethyl-amino;
a compound of Formula (I) wherein A₁ is benzo(1,3)dioxal-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is benzo(1,3)dioxal-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-difluoromethoxy-phenylmethyl, W is N, and Q is 2-amino-4,6-dimethyl-pyridin-3-ylmethyl-amino;
a compound of Formula (I) wherein A₁ is 1-methyl-1H-benzotriazol-5-yl, L₁ is CH₂, D is 4-methoxy-phenylmethyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-(6-amino-pyridin-2-yl)ethyl;
a compound of Formula (I) wherein A₁ is 4-methoxy-phenyl, L₁ is CH₂, D is 5-methoxy-n-pentyl, W is N, and Q is 2-amino-4,6-dimethyl-pyridin-3-ylmethyl-amino;
a compound of Formula (I) wherein A₁ is 4-methoxy-phenyl, L₁ is CH₂, D is 4-methoxy-phenylmethyl, W is CH, and Q is 1-(2-amino-pyridin-4-yl)-ethoxy;
a compound of Formula (I) wherein A₁ is 2,3-dihydro-benzofuran-5-yl, L₁ is CH₂, D is 2,3-dihydro-benzofuran-5-ylmethyl, W is N, and Q is N-oxo-2-amino-4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-difluoromethoxy-phenylmethyl, W is N, and Q is 2-amino-pyridin-3-ylmethyl-amino;
a compound of Formula (I) wherein A₁ is indol-5-yl, L₁ is CH₂, D is 4-methoxy-phenylmethyl, W is N, and Q is 4,6-dimethyl-pyridin-3-ylmethyl-amino;
a compound of Formula (I) wherein A₁ is indol-5-yl, L₁ is CH₂, D is 4-methoxy-phenylmethyl, W is N, and Q is 2-amino-pyridin-3-ylmethyl-amino;
a compound of Formula (I) wherein A₁ is 4-chloro-phenyl, L₁ is CH₂, D is 4-methoxy-phenylmethyl, W is N, and Q is 2-amino-pyridin-3-ylmethyl-amino;
a compound of Formula (I) wherein A₁ is 4-methoxy-phenyl, L₁ is CH₂, D is 4-methoxy-phenylmethyl, W is CH, and Q is 2-amino-pyrimidin-4-ylmethoxy;
and
a compound of Formula (I) wherein A₁ is 2,3-dihydro-benzofuran-5-yl, L₁ is CH₂, D is 4-difluoromethoxy-phenylmethyl, W is N, and Q is N-oxo-2-amino-4,6-dimethyl-pyridin-3-ylmethyl-amino
